(19) United States
(12) Patent Application Publication
(10) Pub. No.: US 2026/0071191 A1
(43) Pub. Date: Mar. 12, 2026

(54) MODIFIED INFLUENZA VIRUSES

(71) Applicant: SEQIRUS INC., Holly Springs, NC (US)

(72) Inventors: Charles MCGEE, Holly Springs, NC (US); Gregory EKBERG, Holly Springs, NC (US); Douglas WIDMAN, Holly Springs, NC (US)

(21) Appl. No.: 19/103,867

(22) PCT Filed: Aug. 16, 2023
(86) PCT No.: PCT/IB2023/058209

§ 371 Date: Feb. 14, 2025

Related U.S. Application Data
(60) Provisional application No. 63/398,362, filed on Aug. 16, 2022.

(51) Int. Cl. C12N 7/00; A61K 31/7088; A61K 39/00; A61K 39/145; C07K 14/11; C12N 15/11

(52) U.S. Cl. CPC C12N 7/00; A61K 31/7088; A61K 39/145; C07K 14/11; C12N 15/11; A61K 2039/525; A61K 2039/53; C12N 2760/16121; C12N 2760/16122; C12N 2760/16134; C12N 2760/16152

(57) ABSTRACT

The present disclosure relates to the field of modified viruses and viral proteins. More particularly, this disclosure relates to modified haemagglutinin proteins, influenza viruses expressing such proteins and methods of making same

### CROSS-REFERENCE TO RELATED APPLICATIONS

The present application claims priority from U.S. Provisional Patent Application No. 63/398,362 filed on 16 Aug. 2022, the contents of which is incorporated herein by reference in its entirety.

Work described in this disclosure was made with United States Government support under Contract HHSO100200900101C, awarded by the Biomedical Advanced Research and Development Authority (BARDA). The Unites States Government may have certain rights in the invention.

### TECHNICAL FIELD

The present disclosure relates to the field of modified viruses and viral proteins. More particularly, this disclosure relates to modified haemagglutinin proteins, influenza viruses expressing such proteins and methods of making same.

### BACKGROUND

Influenza is a major respiratory disease in mammalian species and is responsible for substantial mortality, morbidity and economic losses each year. Three broad types of influenza viruses are recognised, Type A, Type B and Type C, which are defined by the absence of serological cross-reactivity between their internal proteins. Influenza A viruses are further classified into subtypes based on antigenic and genetic differences of their glycoproteins, the haemagglutinin (HA) and neuraminidase (NA) proteins.

More recent vaccine production methods may include the generation and culturing of reassortant influenza viruses in cell culture, such as in Madin Darby Canine Kidney (MDCK) cells. However, certain influenza viruses appear to be less amenable than others to growth and replication under such conditions, which can lead to challenges in vaccine manufacture. Accordingly, there remains a need for the development of genetic modifications to the viral genome of influenza that confer efficient growth and replication to vaccine virus candidates in cell culture.

### SUMMARY

The present disclosure is based on the surprising finding that modifications to amino acid residues of the trimer interface region of a HA protein, particularly a H2 HA protein, can be utilised to improve growth of influenza viruses cultured on cells, such as in Madin Darby Canine Kidney (MDCK) cells. Without being bound by any theory, such modifications may stabilize or increase the stability of the HA trimer of influenza virions whilst in cell culture.

Accordingly, the present disclosure provides a modified haemagglutinin (HA) protein comprising an amino acid sequence wherein one or more amino acid residues of a trimer interface region thereof is modified, wherein an influenza virus expressing the modified HA protein is capable of growth in cells.

Relatedly, the present disclosure also provides a modified HA protein of a H2 sub-type comprising an amino acid sequence wherein one or more amino acid residues thereof is modified at a position selected from the group consisting of V39, L219, V233, V320, K383, I388, N390, K391, V392, S394, A405, R416, D430, F450, and any combination thereof, of a full length H2 amino acid sequence.

In addition, the present disclosure provides a modified HA protein of a H2 sub-type comprising an amino acid sequence wherein one or more amino acid residues thereof is modified in a trimer interface region, and wherein an influenza virus expressing the modified HA protein is capable of growth in cells.

The present disclosure also provides:
an isolated nucleic acid comprising a nucleotide sequence encoding the modified HA protein disclosed herein or a nucleotide sequence complementary thereto;
a genetic construct comprising: (i) the isolated nucleic acid of the present disclosure; or (ii) a nucleotide sequence complementary thereto; operably linked or connected to one or more regulatory sequences; and
a host cell transformed with the isolated nucleic acid of the present disclosure or the genetic construct of the present disclosure.

In addition, the present disclosure provides a method of producing the modified HA protein of the present disclosure, said method including the steps of: (i) culturing the previously transformed host cell of the present disclosure; and (ii) isolating said modified HA protein from said host cell cultured in step (i).

Furthermore, the present disclosure provides an isolated influenza virus, comprising a HA viral segment encoding a modified HA protein comprising an amino acid sequence wherein one or more amino acid residues of a trimer interface region thereof are modified.

In addition, the present disclosure provides a method of preparing an influenza virus in cells, said method including the step of contacting the cells with a genetic construct comprising a nucleic acid that encodes a modified HA protein, wherein the modified HA protein comprises an amino acid sequence in which one or more amino acid residues of a trimer interface region thereof are modified.

The present disclosure further provides an isolated influenza virus prepared by any of the methods disclosed herein; and a modified HA protein prepared by any of the methods disclosed herein.

Relatedly, the present disclosure provides a method of making a vaccine composition, including the steps of:
(a) providing the isolated influenza virus of the present disclosure and/or the modified HA protein of the present disclosure; and
(b) combining the isolated influenza virus and/or the modified HA protein with an adjuvant and/or treating the isolated influenza virus with an agent that inactivates the virus.

Thus, the present disclosure also provides a vaccine composition produced according to the methods disclosed herein of making a vaccine composition.

Similarly, the present disclosure provides a vaccine composition, wherein the vaccine composition comprises:
(a) the isolated influenza virus of the present disclosure and pharmaceutically acceptable carrier, diluent or excipient; or
(b) the modified HA protein of the present disclosure and a pharmaceutically acceptable carrier, diluent or excipient.

Furthermore, the present disclosure provides a method of eliciting an immune response in a subject, said method including the step of administering a therapeutically effective amount of the isolated influenza virus of the present disclosure, the modified HA protein of the present disclosure or the vaccine composition of the present disclosure to the subject to thereby elicit the immune response in the subject.

In addition, the present disclosure provides a method of preventing and/or treating an influenza-associated disease, disorder or condition in a subject, said method including the step of administering a therapeutically effective amount of the isolated influenza virus of the present disclosure, the modified HA protein of the present disclosure or the vaccine composition of the present disclosure to the subject to thereby prevent and/or treat the influenza-associated disease, disorder or condition.

The present disclosure also provides a method of identifying or screening for modifications in a HA protein that facilitate or improve growth of an influenza virus in cells, said method including the steps of:
(a) modifying the influenza virus to express a modified HA protein comprising an amino acid sequence wherein one or more amino acid residues of a trimer interface region thereof is modified; and
(b) testing the ability of the modified influenza virus to grow in cells.

Relatedly, the present disclosure provides a method of identifying modifications in a HA protein that facilitate or improve growth of an influenza virus in cells, said method including the steps of:
(a) modifying the influenza virus to express a modified HA protein comprising an amino acid sequence wherein one or more amino acid residues of a trimer interface region thereof is modified; and
(b) performing one or more passages of the modified influenza virus expressing the modified HA protein in cells.

In addition, the present disclosure provides a method of improving the growth of an influenza virus in cells, said method including the step of modifying the influenza virus to express a modified HA protein comprising an amino acid sequence wherein one or more amino acid residues of a trimer interface region thereof is modified.

The present disclosure also provides a method of improving the stability of an influenza virus strain in cells, said method including the step of modifying the influenza virus strain to express a modified HA protein comprising an amino acid sequence wherein one or more amino acid residues of a trimer interface region thereof is modified.

Furthermore, the present disclosure provides a method of improving manufacture of an influenza virus strain, said method including the step of modifying the influenza virus strain to express a modified HA protein comprising an amino acid sequence wherein one or more amino acid residues of a trimer interface region thereof is modified.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein. It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

FIG. 1. Schematic diagram of candidate vaccine virus (CVV) transfection and rescue.

FIG. 2. Mapping of observed mutations and variants to the structure of A/Swine/Missouri/2124514/2006_HA.

FIG. 3. Outer surface and trimer interface views of HA demonstrating that the HA mutations of the rescue strains of A/Chicken/Ohio/494832/2007 are restricted to the trimer interface region of this molecule.

FIG. 4. A close up structural view of the A405T mutated residue and its proximity to the K423 residue on an adjacent HA monomer.

### KEY TO THE SEQUENCE LISTING

SEQ ID NO: 1 Nucleotide sequence of PB2 viral segment of A/Chicken/Ohio/494832/2007
SEQ ID NO: 2 Nucleotide sequence of PB1 viral segment of A/Chicken/Ohio/494832/2007
SEQ ID NO: 3 Nucleotide sequence of PA viral segment of A/Chicken/Ohio/494832/2007
SEQ ID NO: 4 Nucleotide sequence of HA viral segment of A/Chicken/Ohio/494832/2007
SEQ ID NO: 5 Nucleotide sequence of NP viral segment of A/Chicken/Ohio/494832/2007
SEQ ID NO: 6 Nucleotide sequence of NA viral segment of A/Chicken/Ohio/494832/2007
SEQ ID NO: 7 Nucleotide sequence of M viral segment of A/Chicken/Ohio/494832/2007
SEQ ID NO: 8 Nucleotide sequence of NS viral segment of A/Chicken/Ohio/494832/2007
SEQ ID NO: 9 Amino acid sequence of PB2 protein of A/Chicken/Ohio/494832/2007
SEQ ID NO: 10 Amino acid sequence of PB1 protein of A/Chicken/Ohio/494832/2007
SEQ ID NO: 11 Amino acid sequence of PA protein of A/Chicken/Ohio/494832/2007
SEQ ID NO: 12 Amino acid sequence of HA protein of A/Chicken/Ohio/494832/2007
SEQ ID NO: 13 Amino acid sequence of NP protein of A/Chicken/Ohio/494832/2007
SEQ ID NO: 14 Amino acid sequence of NA protein of A/Chicken/Ohio/494832/2007
SEQ ID NO: 15 Amino acid sequence of M1 protein of A/Chicken/Ohio/494832/2007
SEQ ID NO: 16 Amino acid sequence of M2 protein of A/Chicken/Ohio/494832/2007
SEQ ID NO: 17 Nucleotide sequence of PB2 viral segment of A/Swine/Missouri/2124514/2006
SEQ ID NO: 18 Nucleotide sequence of PB1 viral segment of A/Swine/Missouri/2124514/2006
SEQ ID NO: 19 Nucleotide sequence of PA viral segment of A/Swine/Missouri/2124514/2006
SEQ ID NO: 20 Nucleotide sequence of HA viral segment of A/Swine/Missouri/2124514/2006
SEQ ID NO: 21 Nucleotide sequence of NP viral segment of A/Swine/Missouri/2124514/2006
SEQ ID NO: 22 Nucleotide sequence of NA viral segment of A/Swine/Missouri/2124514/2006
SEQ ID NO: 23 Nucleotide sequence of M viral segment of A/Swine/Missouri/2124514/2006
SEQ ID NO: 24 Nucleotide sequence of NS viral segment of A/Swine/Missouri/2124514/2006
SEQ ID NO: 25 Amino acid sequence of PB2 protein of A/Swine/Missouri/2124514/2006
SEQ ID NO: 26 Amino acid sequence of PB1 protein of A/Swine/Missouri/2124514/2006
SEQ ID NO: 27 Amino acid sequence of PA protein of A/Swine/Missouri/2124514/2006
SEQ ID NO: 28 Amino acid sequence of HA protein of A/Swine/Missouri/2124514/2006
SEQ ID NO: 29 Amino acid sequence of NP protein of A/Swine/Missouri/2124514/2006
SEQ ID NO: 30 Amino acid sequence of NA protein of A/Swine/Missouri/2124514/2006
SEQ ID NO: 31 Amino acid sequence of M1 protein of A/Swine/Missouri/2124514/2006
SEQ ID NO: 32 Amino acid sequence of M2 protein of A/Swine/Missouri/2124514/2006
SEQ ID NO: 33 Amino acid sequence of NS1 protein of A/Swine/Missouri/2124514/2006
SEQ ID NO: 34 Amino acid sequence of NEP protein of A/Swine/Missouri/2124514/2006
SEQ ID NO: 35 Nucleotide sequence of HA viral segment of HS Sys 14 rescue isolate of A/Chicken/Ohio/494832/2007
SEQ ID NO: 36 Amino acid sequence of HA protein of HS Sys 14 rescue isolate of A/Chicken/Ohio/494832/2007
SEQ ID NO: 37 Nucleotide sequence of HA viral segment of HS Sys 15 rescue isolate of A/Chicken/Ohio/494832/2007
SEQ ID NO: 38 Amino acid sequence of HA protein of HS Sys 15 rescue isolate of A/Chicken/Ohio/494832/2007
SEQ ID NO: 39 Nucleotide sequence of HA viral segment of GDE 80.4A rescue isolate of A/Chicken/Ohio/494832/2007
SEQ ID NO: 40 Amino acid sequence of HA protein of GDE 80.4A rescue isolate of A/Chicken/Ohio/494832/2007
SEQ ID NO: 41 Nucleotide sequence of HA viral segment of GDE 80.4B rescue isolate of A/Chicken/Ohio/494832/2007
SEQ ID NO: 42 Amino acid sequence of HA protein of GDE 80.4B rescue isolate of A/Chicken/Ohio/494832/2007
SEQ ID NO: 43 Nucleotide sequence of HA viral segment of GDE-80.7B rescue isolate of A/Chicken/Ohio/494832/2007
SEQ ID NO: 44 Amino acid sequence of HA protein of GDE-80.7B rescue isolate of A/Chicken/Ohio/494832/2007
SEQ ID NO: 45 Nucleotide sequence of HA viral segment of passaged isolate of A/Swine/Missouri/2124514/2006
SEQ ID NO: 46 Amino acid sequence of HA protein of passaged isolate of A/Chicken/Ohio/494832/2007
SEQ ID NO: 47 Nucleotide sequence of HA viral segment of RG4 P2 rescue isolate of A/Swine/Missouri/2124514/2006
SEQ ID NO: 48 Amino acid sequence of HA protein of RG4 P2 rescue isolate of A/Swine/Missouri/2124514/2006
SEQ ID NO: 49 Nucleotide sequence of HA viral segment of RG5 P2 rescue isolate of A/Swine/Missouri/2124514/2006
SEQ ID NO: 50 Amino acid sequence of HA protein of RG5 P2 rescue isolate of A/Swine/Missouri/2124514/2006
SEQ ID NO: 51 Nucleotide sequence of HA viral segment of GDE 80.4A passage 3 replicate A isolate of A/Chicken/Ohio/494832/2007
SEQ ID NO: 52 Amino acid sequence of HA protein of GDE 80.4A passage 3 replicate A isolate of A/Chicken/Ohio/494832/2007
SEQ ID NO: 53 Nucleotide sequence of HA viral segment of GDE 80.4A passage 3 replicate B isolate of A/Chicken/Ohio/494832/2007
SEQ ID NO: 54 Amino acid sequence of HA protein of GDE 80.4A passage 3 replicate B isolate of A/Chicken/Ohio/494832/2007
SEQ ID NO: 55 Nucleotide sequence of HA viral segment of GDE 80.4B passage 3 replicate A isolate of A/Chicken/Ohio/494832/2007
SEQ ID NO: 56 Amino acid sequence of HA protein of GDE 80.4B passage 3 replicate A isolate of A/Chicken/Ohio/494832/2007
SEQ ID NO: 57 Nucleotide sequence of HA viral segment of GDE 80.4B passage 3 replicate B isolate of A/Chicken/Ohio/494832/2007
SEQ ID NO: 58 Amino acid sequence of HA protein of GDE 80.4B passage 3 replicate B isolate of A/Chicken/Ohio/494832/2007
SEQ ID NO: 59 Nucleotide sequence of HA viral segment of GDE-80.7B passage 3 replicate A isolate of A/Chicken/Ohio/494832/2007
SEQ ID NO: 60 Amino acid sequence of HA protein of GDE-80.7B passage 3 replicate A isolate of A/Chicken/Ohio/494832/2007
SEQ ID NO: 61 Nucleotide sequence of HA viral segment of HS_Sys_14 passage 3 replicate A isolate of A/Chicken/Ohio/494832/2007
SEQ ID NO: 62 Amino acid sequence of HA viral segment of HS_Sys_14 passage 3 replicate A isolate of A/Chicken/Ohio/494832/2007
SEQ ID NO: 63 Nucleotide sequence of HA viral segment of HS_Sys_14 passage 3 replicate B isolate of A/Chicken/Ohio/494832/2007
SEQ ID NO: 64 Amino acid sequence of HA viral segment of HS_Sys_14 passage 3 replicate B isolate of A/Chicken/Ohio/494832/2007
SEQ ID NO: 65 Nucleotide sequence of HA viral segment of HS_Sys_15 passage 3 replicate A isolate of A/Chicken/Ohio/494832/2007
SEQ ID NO: 66 Amino acid sequence of HA viral segment of HS_Sys_15 passage 3 replicate A isolate of A/Chicken/Ohio/494832/2007
SEQ ID NO: 67 Nucleotide sequence of HA viral segment of HS_Sys_15 passage 3 replicate B isolate of A/Chicken/Ohio/494832/2007
SEQ ID NO: 68 Amino acid sequence of HA viral segment of HS_Sys_15 passage 3 replicate B isolate of A/Chicken/Ohio/494832/2007
SEQ ID NO: 69 Amino acid sequence of NS1 protein of A/Chicken/Ohio/494832/2007
SEQ ID NO: 70 Amino acid sequence of NEP protein of A/Chicken/Ohio/494832/2007
SEQ ID NO: 71 Amino acid sequence of PB1-F2 protein of A/Chicken/Ohio/494832/2007
SEQ ID NO: 72 Amino acid sequence of PB1-F2 protein of A/Swine/Missouri/2124514/2006
SEQ ID NO: 73 Nucleotide sequence of 5′ non-coding region
SEQ ID NO: 74 Nucleotide sequence of 3′ non-coding region

### DETAILED DESCRIPTION

### General Techniques and Definitions

Unless specifically defined otherwise, all technical and scientific terms used herein shall be taken to have the same meaning as commonly understood by one of ordinary skill in the art (e.g., in genomics, immunology, molecular biology, immunohistochemistry, biochemistry, oncology, and pharmacology).

The present disclosure is performed without undue experimentation using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition, 1995), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment or embodiment of the present disclosure may be applied mutatis mutandis to any other aspect or embodiment or embodiment of the present disclosure.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

As used herein, the singular forms of “a”, “and” and “the” include plural forms of these words, unless the context clearly dictates otherwise. For example, a reference to “a bacterium” includes a plurality of such bacteria, and a reference to “an allergen” is a reference to one or more allergens.

The term “and/or”, e.g., “X and/or Y” shall be understood to mean either “X and Y” or “X or Y” and shall be taken to provide explicit support for both meanings or for either meaning.

Throughout this specification, the word “comprise’ or variations such as “comprises” or “comprising” will be understood to imply the inclusion of a stated element, integer or step, or group of elements, integers or steps, but not the exclusion of any other element, integer or step, or group of elements, integers or steps.

By “consisting essentially of”, in the context of an amino acid sequence, is meant the recited amino acid sequence together with an additional one, two or three amino acids at the N- or C-terminus.

The term “substantially” does not exclude “completely” (e.g., a composition which is “substantially free” from Y may be completely free from Y).

The term “about” in relation to a numerical value x is optional and means, for example, any number within 1, 5 or 10% of the referenced number. In certain examples, the term “about” encompasses the exact number recited.

All computer programs, algorithms, patent and scientific literature referred to herein is incorporated herein by reference.

For the present disclosure, the database accession number or unique identifier provided herein for a gene, protein or virus strain, as well as the gene and/or protein sequence or sequences associated therewith, are incorporated by reference herein.

### Modified HA Proteins

The inventors have surprisingly shown that modifying particular amino acid residues of the trimer interface region of a H2 protein can facilitate or improve the growth of influenza viruses expressing the modified haemagglutinin (HA) protein in cell culture. These modified proteins may also increase virus yield, which may be advantageous for vaccine production.

Accordingly, in one form, the present disclosure provides a modified haemagglutinin (HA) protein comprising an amino acid sequence, wherein one or more amino acid residues of a trimer interface region thereof are modified. Suitably, an influenza virus expressing the modified HA protein is capable of growth in cells. In this regard, the influenza virus when expressing an unmodified or wild-type form of the modified HA protein is suitably not capable of growth in cells or has a limited or reduced capability for growth in cells. Accordingly, the modified HA protein suitably provides the influenza virus expressing the HA protein with a capability of growth in cells or an improved capability of growth in cells.

In a related form, the present disclosure provides a modified HA protein of a H2 sub-type comprising an amino acid sequence wherein one or more amino acid residues of a trimer interface region thereof are modified. In some examples, the one or more amino acid residues are modified at a position selected from the group consisting of 39, 219, 233, 320, 383, 388, 390, 391, 392, 394, 405, 416, 430, 450 and any combination thereof, of a full length H2 amino acid sequence. More particularly, the one or more amino acid residues can be modified at a position selected from the group consisting of V39, L219, V233, V320, K383, I388, N390, K391, V392, S394, A405, R416, D430, F450 and any combination thereof, of a full length H2 amino acid sequence (for example, as set out in SEQ ID NO: 12).

In another related form, the present disclosure provides a modified HA protein of a H2 sub-type comprising an amino acid sequence wherein one or more amino acid residues thereof is modified in a trimer interface region, and wherein an influenza virus expressing the modified HA protein is capable of growth in cells.

Influenza haemagglutinin (HA) is a glycoprotein encoded by the HA gene segment of an influenza virus. HA is typically expressed as a homotrimer on the surface of the viral capsid and is integral to its infectivity. To this end, HA allows for the recognition of cells in the upper respiratory tract or erythrocytes by binding to glycans thereon that contain the monosaccharide sialic acid. This leads to internalisation of the influenza virus by the cell into an endosome, which subsequently facilitates conformational rearrangement of the HA trimer. The HA protein then fuses with the endosomal membrane, thereby allowing for the release of viral gene segments, which are in the form of a ribonucleoprotein complex (RNP) together with nucleoproteins and a polymerase complex, into the cytoplasm of the host cell. The RNPs are transported into the host nucleus followed by transcription, and replication of the viral genome. The HA protein, together with the other newly generated viral proteins and a replicated genome, is then incorporated into the envelope of an influenza virion as it buds from an infected host cell. The HA protein on new viral particles remains attached to sialic acid groups of glycoproteins on the external cell surface and neuraminidase (NA) cleaves these groups and thereby allows for the efficient release of the newly formed virions.

The terms “haemagglutinin”, “hemagglutinin” and “HA” refer to any haemagglutinin protein known to those of skill in the art (e.g., influenza A HA subtypes of H1, H2, H3, H4, H5, H6, H7, H8, H9, H10, H11, H12, H13, H14, H15 and H16). In particular examples, however, the modified HA protein described herein is of a H2, H1, H5, H3, H7 or H9 influenza A subtype or more particularly of a H2, H1 or H5 influenza A subtype. In some examples, the modified HA protein described herein is of a H2 influenza A subtype. The HA may also be derived from an influenza virus isolate from any host species. In various examples, the modified HA protein is derived at least partly from an avian influenza virus isolate or strain. In other examples, the modified HA protein is derived at least partly from a swine influenza virus isolate or strain.

According to certain examples, the modified HA protein provided herein is an influenza haemagglutinin protein, such as an influenza A haemagglutinin protein or an influenza B haemagglutinin protein. A typical haemagglutinin protein comprises a signal peptide, a stem or stalk domain, a globular head domain, a luminal domain, a transmembrane domain and a cytoplasmic domain. In some examples, the modified haemagglutinin protein provided herein comprises a single polypeptide chain, such as HA0, HA1 or HA2. In other examples, the modified HA protein comprises more than one polypeptide chain in quaternary association (e.g., HA1 and HA2). In other examples, the modified haemagglutinin protein lacks a signal peptide (i.e., the modified haemagglutinin protein is a mature haemagglutinin). In alternative examples, the modified haemagglutinin protein comprises a signal peptide (i.e., the modified haemagglutinin protein is a full length haemagglutinin; e.g., as set out in SEQ ID NO: 12). The modified haemagglutinin proteins provided herein may also be further modified by post-translational processing, such as signal peptide cleavage, disulfide bond formation, glycosylation (e.g., N-linked glycosylation), protease cleavage and lipid modification (e.g., S-palmitoylation).

Insofar as it relates to a HA protein, the term “trimer interface region” as used herein refers to an outer or surface region of a HA monomer that may associate, interact, contact or bind with an adjacent HA monomer upon formation of a HA trimer. In this regard, the one or more modified amino acid residues may be adjacent and/or face towards the trimer interface region of a further HA molecule or monomer, such as when in a dimer or trimer arrangement. Furthermore, the one or more modified amino acid residues may modulate a structural feature, such as a tertiary structure, of the trimer interface region. Suitably, the trimer interface region comprises one or more amino acid residues that approach and participate in an interaction with one or more amino acid residues on an adjacent HA monomer upon trimer formation. Such interactions may include, for example, hydrogen bonding, electrostatic interactions, salt bridges, and the like. In particular examples, the one or more modified amino acid residues of the trimer interface region are within or less than about 15 angstroms (e.g., within about 15, 14, 13, 12, 11, 10, 9.5, 9, 8.5, 8, 7.5, 7, 6.5, 6, 5.5, 5, 4.5, 4, 3.5, 3, 2.5, 2, 1.5, 1 or 0.5 angstroms or any range therein) from one or more residues in an adjacent HA monomer upon trimer formation. In other examples, the one or more modified amino acid residues of the trimer interface region are within or less than about 11 angstroms from one or more residues in an adjacent HA monomer upon trimer formation. In various examples, the one or more modified amino acid residues of the trimer interface region are within or less than about 8.5 angstroms from one or more residues in an adjacent HA monomer upon trimer formation.

It is further contemplated that amino acid residues of a trimer interface region can be found in any portion, subunit or domain of a HA protein or molecule, such as in the globular head domain and/or the stalk domain. In particular examples, one or more of the modified amino acids are present in a lower region of the stalk domain (e.g., one or more of residues V39, K383, I388, N390, K391, V392, S394, and F450 of a full length H2 HA protein). Suitably, the trimer interface region in the lower region of the stalk domain may comprise, consist of or consist essentially of amino acid residues at positions 377 to 397, 439 to 452, 26 to 46 and, optionally, 325 to 335, of a full length HA protein of a H2 subtype (e.g. as those positions are indicated in SEQ ID NO: 12). Alternatively, the trimer interface region in the lower region of the stalk domain may comprise, consist of or consist essentially of amino acid residues at positions 383 to 394, 439 to 452, 31 to 40 and, optionally, 325 to 335, of a full length HA protein of a H2 subtype (e.g. as those positions are indicated in SEQ ID NO: 12). In a further alternative, the trimer interface region in the lower region of the stalk domain may comprise, consist of or consist essentially of amino acid residues at positions 26 to 46, 325 to 335, 377 to 397 and 439 to 452 (e.g., N26 to D46, L325 to P335, D377 to E397 and L439 to D452) of a full length HA protein of a H2 subtype (e.g., as set out in SEQ ID NO: 12 or SEQ ID NO: 28). In particular examples, the trimer interface region in the lower region of the stalk domain comprises, consists of or consists essentially of amino acid residues at positions 26 through 46, 377 through 397 and 439 through 452 (e.g., N26 through D46, D377 through E397 and L439 through D452) of a full length HA protein of a H2 subtype.

Suitably, the modified HA protein comprises a modification to one or more amino acid residues at a position selected from the group consisting of 39, 383, 388, 390, 391, 392, 394, 450 and any combination thereof, of a full length H2 amino acid sequence (e.g., as set out in SEQ ID NO: 12 or SEQ ID NO: 28). In some examples, the modified HA protein comprises an isoleucine modification at amino acid residue position 39 of a full length H2 amino acid sequence. For other examples, the modified HA protein comprises a glutamate modification at amino acid residue position 383 of a full length H2 amino acid sequence. Referring to certain examples, the modified HA protein comprises a threonine modification at amino acid residue position 388 of a full length H2 amino acid sequence. In particular examples, the modified HA protein comprises an isoleucine modification at amino acid residue position 390 of a full length H2 amino acid sequence. For other examples, the modified HA protein comprises an arginine or an asparagine modification at amino acid residue position 391 of a full length H2 amino acid sequence. Referring to various examples, the modified HA protein comprises a alanine modification at amino acid residue position 392 of a full length H2 amino acid sequence. In some examples, the modified HA protein comprises a tyrosine modification at amino acid residue position 394 of a full length H2 amino acid sequence. For other examples, the modified HA protein comprises a serine modification at amino acid residue position 450 of a full length H2 amino acid sequence.

More particularly, the modifications to the one or more amino acid residues of the modified HA protein are suitably selected from the group consisting of 39I, 383E, 388T, 390I, 391R, 391N, 392A, 394Y, 450S and any combination thereof, of a full length H2 amino acid sequence (e.g., as set out in SEQ ID NO: 12 or SEQ ID NO: 28). Even more particularly, in some examples, the modifications to the one or more amino acid residues of the modified HA protein are selected from the group consisting of V39I, K383E, I388T, N390I, K391R, K391N, V392A, S394Y, F450S and any combination thereof, of a full length H2 amino acid sequence (e.g., as set out in SEQ ID NO: 12 or SEQ ID NO: 28).

In this regard, the trimer interface region may be divided into a plurality of non-contiguous portions of an amino acid sequence of a HA monomer. By way of example, one or more amino acid residues of the trimer interface region may be found in a HA1 subunit and one or more further amino acid residues of the trimer interface region may be found in a HA2 subunit of a HA protein. Additionally, one or more amino acid residues of the trimer interface region may be found in a stem domain and one or more further amino acid residues of the trimer interface region may be found in a globular head domain of a HA protein.

The term “modified protein”, e.g., “modified HA protein”, is to be understood as a protein which contains one or a plurality of modifications compared to a parent, consensus or wild-type protein, such as a wild-type HA protein. Wild type HA protein sequences can be determined experimentally or are publicly available on a number of databases. A suitable example is provided in the present disclosure as SEQ ID NO: 12. Another example is provided herein as SEQ ID NO: 28. The term “modification” or “modified” in the context of the present disclosure is to be understood as to include chemical modification of a protein as well as genetic manipulation of the DNA encoding a protein. Such modifications may be replacement of one or more amino acid side chains, one or more substitutions, one or more deletions and/or one or more insertions in the protein of interest. Additionally, these terms are envisaged to include the screening and/or selection of existing influenza virus isolates having or expressing a HA protein that includes one or more amino acid residues that are described herein as “modified” (e.g., V39, L219, V233, V320, K383, I388, N390, K391, V392, S394, A405, R416, D430 and F450; these positions being numbered in accordance with the conventional numbering system of a full length HA protein, such as that exemplified in SEQ ID NO: 12 or SEQ ID NO: 28).

Suitably, the modified HA protein described herein exhibits or possesses altered or modulated stability (e.g., increased stability), such as in a trimer formation thereof, as compared to a wild-type or unmodified HA protein of a corresponding influenza virus isolate. As such, the phrase “increasing the stability of a HA trimer” or the like can mean that after being modified in accordance with the present disclosure, the modified HA protein is able to form a more stable homotrimer arrangement when present in cell culture, in comparison to the starting (unmodified) HA protein. Without being bound by any theory, the modifications to the trimer interface region described herein may function to improve the ability of the modified HA protein monomers to form and maintain a homotrimeric arrangement, such as when influenza virus isolates expressing such modified HA proteins are grown in cell culture. Accordingly, in particular examples, the modified HA protein is capable of forming a HA trimer when an influenza virus isolate expressing the modified HA protein is grown in cell culture. In this regard, the interface region of the modified HA protein is suitably capable of interacting with one or more further HA protein monomers to form a stable trimer arrangement thereof. In some examples, the modified HA protein is capable of forming a HA trimer having increased stability when compared to a wild-type or unmodified HA protein (e.g., a HA protein that does not include the respective modifications to the one or more amino acid residues of the interface region of the modified HA protein).

As such, in particular examples, HA trimers containing the modified HA protein described herein exhibit increased stability as compared to a corresponding HA trimer containing wild-type or unmodified HA proteins. By “enhanced”, “increased” or “up regulated” as used herein to describe the stability of the modified HA protein or a trimer thereof, refers to the increase in a level of stability when compared to that of a control or reference sample (e.g., a HA trimer containing a wild-type or unmodified HA protein that does not include one or more of the modifications described herein).

Stability of the modified HA protein, or more particularly homotrimers thereof, may be assessed by any means in the art, and may be assessed directly or indirectly, such as by rescue capability, growth capability and/or replication capability thereof when grown in cell culture, such as in mammalian cells like MDCK cells. As such, the phrase “increasing the stability of a HA trimer” or the like can mean that a virus having a modified HA protein in accordance with the present disclosure has an improved rescue capability, growth capability and/or replication capability when grown in cell culture (such as in mammalian cells like MDCK cells), compared to a virus having an unmodified or wild type HA protein. Thus, the term “stability” can also be used in the context of a virus having a modified HA protein in accordance with the present disclosure. For example, a virus having a modified HA protein in accordance with the present disclosure can have an increased or improved stability, in that it can have an improved rescue capability, growth capability and/or replication capability when grown in cell culture (such as in mammalian cells like MDCK cells), compared to a virus having an unmodified or wild type HA protein. The extent of the improvement in any one or more of these capabilities (e.g., rescue capability, growth capability and/or replication capability) may vary. For example, the extent of the improvement, such as in rescue capability, growth capability and/or replication capability, may be more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% higher than that level observed in an influenza virus strain expressing a control HA protein (e.g., an unmodified HA protein, or a wild-type HA protein). For example, the extent of the improvement may be more than about 50% compared to a virus having or expressing an unmodified or wild type HA protein. In another example, the improved rescue capability, growth capability and/or replication capability may be determined by achieving rescue of a threshold level of replicated virus grown on MDCK cells that allows production of a quantity of virus for use in the preparation of a vaccine at a commercial scale.

Further exemplary methods of assessing the stability of the modified HA protein or homotrimers thereof may include computational or 3D modelling, co-immunoprecipitation, pull down assays and far-western assays. In this regard, the modified HA protein may be contained in a live or attenuated virus or in a virus-like particle (VLP).

In any of the methods used to detect stability of the modified HA protein or a homotrimer thereof, the level of stability of the modified HA protein may be relative or absolute. In some examples, the level of stability of the modified HA protein is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% higher than that level observed in a control HA protein (e.g., an unmodified HA protein, or a wild-type HA protein, such as SEQ ID NO: 12 or SEQ ID NO: 28).

The modified HA protein described herein may be considered to be isolated. For the purposes of the present disclosure, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

By “protein” is meant an amino acid polymer. The amino acids may be natural or non-natural amino acids, D- or L-amino acids as are well understood in the art.

The term “protein” includes and encompasses “peptide”, which is typically used to describe a protein having no more than fifty (50) amino acids (e.g., no more than 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids and any range therein) and “polypeptide”, which is typically used to describe a protein having more than fifty (50) amino acids.

In particular examples, the modified HA protein includes about 500 to about 600 amino acid residues, more particularly about 510 to about 590 acid residues, even more particularly about 520 to about 580 amino acid residues, yet even more particularly about 530 to about 570 amino acid residues or still even more particularly about 540 to about 570 amino acid residues (e.g., about 540, 541, 542, 543, 544, 545, 546, 547, 548, 549, 550, 551, 555, 553, 554, 555, 556, 557, 558, 559, 560, 561, 562, 563, 564, 565, 566, 567, 568, 569, 570 amino acid residues or any range therein).

It is contemplated that the modified HA protein described herein suitably does not include or constitute an amino acid sequence of a wild-type HA protein, such as a wild-type HA protein of the H2 subtype. In particular examples, the modified HA protein described herein shares at least 70% or 75%, more particularly at least 80% or 85% or even more particularly at least 90%, 91%, 92%, 93%, 94%, 95%, 96%, 97%, 98% or 99% sequence identity with a reference, unmodified or wild-type amino acid sequence of a HA protein, such as that set forth in SEQ ID NO: 12, or alternatively, SEQ ID NO: 28.

Variants of the modified HA proteins described herein are envisaged by the present disclosure. As used herein, a protein, polypeptide or peptide “variant” shares a definable amino acid sequence relationship with a reference amino acid sequence. In particular examples, the reference amino acid sequence is that of a wild-type HA protein or a modified HA protein, inclusive of a mature amino acid sequence (i.e., no signal sequence) and an amino acid sequence which includes the signal sequence (i.e., a full length amino acid sequence). The reference amino acid sequence may be the amino acid sequence of any one of SEQ ID NOs: 12 or 28, for example. The “variant” protein, polypeptide or peptide may have one or a plurality of amino acids of the reference amino acid sequence deleted or substituted by different amino acids. Such modified HA protein variants may include, for example, amino acid residues and/or amino acid sequences of naturally occurring variants and orthologs (e.g., from a different influenza strain or a different host animal) of a HA protein, inclusive of a consensus sequence thereof. In this regard, it is envisaged that one or more of the amino acid variations described herein (e.g., V39I, L219P, V233A, V320A, V320I, K383E, I388T, N390I, K391R, K391N, V392A, S394Y, A405T, R416G, D430N and F450S of a full length HA protein of a H2 subtype) may be introduced into any HA protein as are known in the art. It is further contemplated that some amino acids may be substituted or deleted without changing the activity of the variant protein (i.e., conservative substitutions). Accordingly, one or more of the residues of the modified HA protein described herein, such as those defined by SEQ ID NOs: 12 or 28, may be conservatively modified (e.g., by amino acid substitution or deletion) so as to substantially retain the functionality and/or immunogenicity of the modified HA protein. Furthermore, the modified HA protein may include additional amino acid variations that are outside of the trimer interface region, such as in one or more hypervariable regions thereof (e.g., a hypervariable head region) as are known in the art. Such additional amino acid variations may include for example variations at positions V129 (e.g., V129I) and/or E184 (e.g., E184K) of a full length H2 HA protein, as described herein.

It is envisaged that protein or peptide variants share at least 70% or 75%, more particularly at least 80% or 85% or even more particularly at least 90%, 91%, 92%, 93%, 94%, 95%, 96%, 97%, 98% or 99% sequence identity with a reference amino acid sequence, such as that set forth in SEQ ID NO: 12, or alternatively, SEQ ID NO: 28.

Terms used generally herein to describe sequence relationships between respective proteins and nucleic acids include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of, for example, 6, 9, 12 or 20 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, WI, USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1991, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) or amino acid residue occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, California, USA).

It is envisaged that the trimer interface region may be modified at any amino acid residue therein as is known in the art. The modifications described herein may include, but are not limited to, deletions, additions and substitutions in the amino acid sequence of the HA protein in question. For example, one class of substitutions is a conserved amino acid substitution. Such substitutions are those that substitute a given amino acid in a HA protein by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent can be found in, for example, Bowie et al., Science 247:1306-1310 (1990).

In certain examples, the modified HA protein includes the deletion of one or more amino acid residues of the trimer interface region, such as those residues described herein. The term “deletion” refers to the removal of one or more (or a specified number of) contiguous amino acids from a respective peptide, polypeptide or protein.

According to particular examples, the modified HA protein includes the mutation or substitution of one or more (e.g., 1, 2, 3, 4, 5, 6, 7, 8, 9, 10 etc) amino acid residues of the trimer interface region, such as those residues described herein. As used herein, “substituted” and “substitutions” refer to replacement(s) of an amino acid residue in a parent or reference sequence, such as SEQ ID NO: 12, or alternatively, SEQ ID NO: 28. In some examples, the substitution involves the replacement of a naturally occurring or conserved residue. The modified HA protein herein encompasses the substitution of one or more amino acid residues of the trimer interface region, as described herein, by any one of the remaining nineteen amino acids. In particular examples, the modified HA protein comprises a substitution of an amino acid of one size to an amino acid of a different size (e.g., substitution of an amino acid with an amino acid of a relatively larger size or a relatively smaller size), an amino acid of one hydrophilicity to an amino acid of a different hydrophilicity, an amino acid of one polarity to an amino acid of a different polarity and/or an amino acid of one acidity to an amino acid of a different acidity. The modification in the modified HA protein may be or include a substitution of an amino acid at a specific location identified in any of the examples herein with the respective, substitute amino acid identified in any of the examples herein.

As noted above, the modified HA protein can be of a H2 subtype. In this context, and according to particular examples, the one or more amino acid residue positions of the trimer interface region that are modified, such as by substitution, in the modified HA protein are selected from the group consisting of 39, 219, 233, 320, 383, 388, 390, 391, 392, 394, 405, 416, 430, 450 and any combination thereof (e.g., V39, L219, V233, V320, K383, I388, N390, K391, V392, S394, A405, R416, D430, F450 and any combination thereof), wherein the amino acid numbering is based on a full length H2 amino acid sequence (e.g., an amino acid sequence set forth in SEQ ID NO: 12 or SEQ ID NO: 28; i.e., the amino acid numbering is based on the first methionine (methionine in position 1; M1) being the first residue). In other examples, the one or more amino acid residues of the trimer interface region that are modified are selected from the group consisting of V39, K383, I388, N390, K391, V392, S394, F450 and any combination thereof, wherein the amino acid numbering is based on a full length H2 amino acid sequence. According to some examples, the modified HA protein includes one or more of the substitutions or mutations of 39I, 219P, 233A, 320A, 320I, 383E, 388T, 390I, 391R, 391N, 392A, 394Y, 405T, 416G, 430N and 450S according to the amino acid numbering of a full length H2 HA protein (e.g., SEQ ID NO: 12 or SEQ ID NO: 28). In particular examples, the modified HA protein includes one or more of the substitutions or mutations of V39I, L219P, V233A, V320A, V320I, K383E, I388T, N390I, K391R, K391N, V392A, S394Y, A405T, R416G, D430N and F450S according to the amino acid numbering of a full length H2 HA protein (e.g., SEQ ID NO: 12 or SEQ ID NO: 28). For certain examples, the modified HA protein includes one or more of the substitutions or mutations of 39I, 383E, 388T, 390I, 391R, 391N, 392A, 394Y and 450S, according to the amino acid numbering of a full length H2 HA protein (e.g., SEQ ID NO: 12 or SEQ ID NO: 28). In various examples, the modified HA protein includes one or more of the substitutions or mutations of V39I, K383E, I388T, N390I, K391R, K391N, V392A, S394Y and F450S, according to the amino acid numbering of a full length H2 HA protein (e.g., SEQ ID NO: 12 or SEQ ID NO: 28).

According to certain examples, the modified HA protein comprises, consists of or consists essentially of an amino acid sequence selected from SEQ ID NOs: 36, 38, 40, 42, 44, 46, 48, 50, 52, 54, 56, 58, 60, 62, 64, 66 or 68, or a fragment, variant or derivative thereof.

Suitably, the modified HA protein comprises a modification, such as a substitution, at a position corresponding to position 405 of a full length H2 HA protein. More particularly, the modified HA protein suitably comprises a modification, such as a substitution, of alanine at a position corresponding to position 405 of a full length H2 HA protein. In various examples, the modified HA protein comprises a substitution of threonine instead of alanine at a position corresponding to position 405 (i.e., A405T) of a full length H2 HA protein. More particularly, the modified HA protein may comprise, consist of or consist essentially of an amino acid sequence set forth in SEQ ID NO: 38, or a fragment, variant or derivative thereof. Influenza virus isolates (e.g., reassortant influenza viruses) expressing such a modified HA protein may be particularly suitable or suited to large scale manufacturing thereof.

Suitably, the modified HA protein comprises a modification, such as a substitution, at a position corresponding to position 39 of a full length H2 HA protein. More particularly, the modified HA protein suitably comprises a modification, such as a substitution, of valine at a position corresponding to position 39 of a full length H2 HA protein. In certain examples, the modified HA protein comprises a substitution of isoleucine instead of valine at a position corresponding to position 39 (i.e., V39I) of a full length H2 HA protein. More particularly, the modified HA protein may comprise, consist of or consist essentially of an amino acid sequence set forth in SEQ ID NO: 42, or a fragment, variant or derivative thereof.

Suitably, the modified HA protein comprises a modification, such as a substitution, at a position corresponding to position 394; and/or a position corresponding to position 416; of a full length H2 HA protein. More particularly, the modified HA protein suitably comprises a modification, such as a substitution, of one or more of: serine at a position corresponding to position 394; and arginine at a position corresponding to position 416; of a full length H2 HA protein. In certain examples, the modified HA protein comprises a substitution of:
(a) tyrosine instead of serine at a position corresponding to position 394 (i.e., S394Y); and/or
(b) alanine instead of arginine at a position corresponding to position 416 (i.e., R416G);
of a full length H2 HA protein.

In some examples, the modified HA protein comprises a modification of one or more amino acid residues at position: (a) S394; (b) R416; or (c) S394 and R416; of a full length H2 HA protein. In other examples, the modified HA protein comprises a modification of: (a) S394Y; (b) R416G; or (c) S394Y and R416G; of a full length H2 HA protein. More particularly, the modified HA protein may comprise, consist of or consist essentially of an amino acid sequence set forth in SEQ ID NO: 36, or a fragment, variant or derivative thereof. Again, influenza virus isolates (e.g., reassortant influenza viruses) expressing such a modified HA protein may be particularly suitable or suited to large scale manufacturing thereof.

Suitably, the modified HA protein comprises a modification, such as a substitution, at a position corresponding to position 233; and/or a position corresponding to position 320; of a full length H2 HA protein. More particularly, the modified HA protein suitably comprises a modification, such as a substitution, of one or more of: valine at a position corresponding to position 233; and valine at a position corresponding to position 320; of a full length H2 HA protein. In certain examples, the modified HA protein comprises a substitution of:
(a) alanine instead of valine at a position corresponding to position 233 (i.e., V233A); and/or
(b) alanine instead of valine at a position corresponding to position 320 (i.e., V320A);
of a full length H2 HA protein.

In some examples, the modified HA protein comprises a modification of one or more amino acid residues at position: (a) V233; (b) V320; or (c) V233 and V320; of a full length H2 HA protein. In other examples, the modified HA protein comprises a modification of: (a) V233A; (b) V320A; or (c) V233A and V320A; of a full length H2 HA protein. More particularly, the modified HA protein may comprise, consist of or consist essentially of an amino acid sequence set forth in SEQ ID NO: 40, or a fragment, variant or derivative thereof.

Suitably, the modified HA protein comprises a modification, such as a substitution, at a position corresponding to position 383 of a full length H2 HA protein. More particularly, the modified HA protein suitably comprises a modification, such as a substitution, of lysine at a position corresponding to position 383 of a full length H2 HA protein. In various examples, the modified HA protein comprises a substitution of glutamate instead of lysine at a position corresponding to position 383 (i.e., K383E) of a full length H2 HA protein. More particularly, the modified HA protein may comprise, consist of or consist essentially of an amino acid sequence set forth in SEQ ID NO: 48, or a fragment, variant or derivative thereof.

Suitably, the modified HA protein comprises a modification, such as a substitution, at a position corresponding to position 320, at a position corresponding to position 390, and/or at a position corresponding to position 391, of a full length H2 HA protein. More particularly, the modified HA protein suitably comprises a modification, such as a substitution, of one or more of valine at a position corresponding to position 320; asparagine at a position corresponding to position 390; and, lysine at a position corresponding to position 391; of a full length H2 HA protein. In certain examples, the modified HA protein comprises a substitution of
(a) isoleucine instead of valine at a position corresponding to position 320 (i.e., V320I);
(b) isoleucine instead of asparagine at a position corresponding to position 390 (i.e., N390I); and/or
(c) arginine instead of lysine at a position corresponding to position 391 (i.e., K391R);
of a full length H2 HA protein.

In some examples, the modified HA protein comprises a modification of one or more amino acid residues at position: (a) V320; (b) N390; (c) K391; (d) V320 and N390; (e) V320 and K391; (f) N390 and K391; or (g) V320, N390 and K391; of a full length H2 HA protein. In other examples, the modified HA protein comprises a modification of: (a) V320I; (b) N390I; (c) K391R; (d) V320I and N390I; (e) V320I and K391R; (f) N390I and K391R; or (g) V320I, N390I and K391R; of a full length H2 HA protein. More particularly, the modified HA protein may comprise, consist of or consist essentially of an amino acid sequence set forth in SEQ ID NO: 46, or a fragment, variant or derivative thereof.

Suitably, the modified HA protein comprises a modification, such as a substitution, at a position corresponding to position 219 of a full length H2 HA protein. More particularly, the modified HA protein suitably comprises a modification, such as a substitution, of leucine at a position corresponding to position 219 of a full length H2 HA protein. In various examples, the modified HA protein comprises a substitution of proline instead of leucine at a position corresponding to position 219 (i.e., L219P) of a full length H2 HA protein. More particularly, the modified HA protein may comprise, consist of or consist essentially of an amino acid sequence set forth in SEQ ID NO: 50, or a fragment, variant or derivative thereof.

Suitably, the modified HA protein comprises a modification, such as a substitution, at a position corresponding to position 450 and/or at a position corresponding to position 391 of a full length H2 HA protein. More particularly, the modified HA protein suitably comprises a modification, such as a substitution, of one or more of: phenylalanine at a position corresponding to position 450; and lysine at a position corresponding to position 391; of a full length H2 HA protein. In certain examples, the modified HA protein comprises a substitution of:
(a) alanine instead of valine at a position corresponding to position 233 (i.e., V233A);
(b) serine instead of phenylalanine at a position corresponding to position 450 (i.e., F450S); and/or
(c) asparagine instead of lysine at a position corresponding to position 391 (i.e., K391N);
of a full length H2 HA protein.

In some examples, the modified HA protein comprises a modification of one or more amino acid residues at position: (a) V233; (b) F450; (c) K391; (d) V233 and F450; (e) V233 and K391; (f) F450 and K391; or (g) V233, F450 and K391; of a full length H2 HA protein. In other examples, the modified HA protein comprises a modification of: (a) V233A; (b) F450S; (c) K391N; (d) V233A and F450S; (e) V233A and K391N; (f) F450S and K391N; or (g) V233A, F450S and K391N; of a full length H2 HA protein. More particularly, the modified HA protein may comprise, consist of or consist essentially of an amino acid sequence set forth in SEQ ID NO: 54, or a fragment, variant or derivative thereof.

Suitably, the modified HA protein comprises a modification, such as a substitution, at a position corresponding to position 450 and/or at a position corresponding to position 391 of a full length H2 HA protein. More particularly, the modified HA protein suitably comprises a modification, such as a substitution, of one or more of: phenylalanine at a position corresponding to position 450; and lysine at a position corresponding to position 391; of a full length H2 HA protein. In certain examples, the modified HA protein comprises a substitution of:
(a) serine instead of phenylalanine at a position corresponding to position 450 (i.e., F450S); and/or
(b) asparagine instead of lysine at a position corresponding to position 391 (i.e., K391N);
of a full length H2 HA protein.

In some examples, the modified HA protein comprises a modification of one or more amino acid residues at position: (a) F450; (b) K391; or (c) F450 and K391; of a full length H2 HA protein. In other examples, the modified HA protein comprises a modification of: (a) F450S; (b) K391N; or (c) F450S and K391N; of a full length H2 HA protein. More particularly, the modified HA protein may comprise, consist of or consist essentially of an amino acid sequence set forth in SEQ ID NO: 52, or a fragment, variant or derivative thereof.

Suitably, the modified HA protein comprises a modification, such as a substitution, at a position corresponding to position 39 and/or at a position corresponding to position 430, of a full length H2 HA protein. More particularly, the modified HA protein suitably comprises a modification, such as a substitution, of one or more of: valine at a position corresponding to position 39; aspartate at a position corresponding to position 430; of a full length H2 HA protein. In certain examples, the modified HA protein comprises a substitution of:
(a) isoleucine instead of valine at a position corresponding to position 39 (i.e., V39I); and/or
(b) asparagine instead of aspartate at a position corresponding to position 430 (i.e., D430N);
of a full length H2 HA protein.

In some examples, the modified HA protein comprises a modification of one or more amino acid residues at position: (a) V39; (b) D430; or (c) V39 and D430; of a full length H2 HA protein. In other examples, the modified HA protein comprises a modification of: (a) V39I; (b) D430N; or (c) V39I and D430N; of a full length H2 HA protein. More particularly, the modified HA protein may comprise, consist of or consist essentially of an amino acid sequence set forth in SEQ ID NO: 56, or a fragment, variant or derivative thereof.

Suitably, the modified HA protein comprises a modification, such as a substitution, at a position corresponding to position 39, at a position corresponding to position 388, and/or at a position corresponding to position 392, of a full length H2 HA protein. More particularly, the modified HA protein suitably comprises a modification, such as a substitution, of one or more of: valine at a position corresponding to position 39; isoleucine at a position corresponding to position 388; and valine at a position corresponding to position 392; of a full length H2 HA protein. In certain examples, the modified HA protein comprises a substitution of:
(a) isoleucine instead of valine at a position corresponding to position 39 (i.e., V39I);
(b) threonine instead of isoleucine at a position corresponding to position 388 (i.e., I388T); and/or
(c) alanine instead of valine at a position corresponding to position 392 (i.e., V392A);
of a full length H2 HA protein.

In some examples, the modified HA protein comprises a modification of one or more amino acid residues at position: (a) V39; (b) I388; (c) V392; (d) V39 and I388; (e) V39 and V392; (f) I388 and V392; or (g) V39, I388 and V392; of a full length H2 HA protein. In other examples, the modified HA protein comprises a modification of: (a) V39I; (b) I388T; (c) V392A; (d) V39I and I388T; (e) V39I and V392A; (f) I388T and V392A; or (g) V39I, I388T and V392A; of a full length H2 HA protein. More particularly, the modified HA protein may comprise, consist of or consist essentially of an amino acid sequence set forth in SEQ ID NO: 58, or a fragment, variant or derivative thereof.

Suitably, the modified HA protein comprises a modification, such as a substitution, at a position corresponding to position 39, at a position corresponding to position 391, and/or at a position corresponding to position 430, of a full length H2 HA protein. More particularly, the modified HA protein suitably comprises a modification, such as a substitution, of one or more of: valine at a position corresponding to position 39; lysine at a position corresponding to position 391; and aspartate at a position corresponding to position 430; of a full length H2 HA protein. In certain examples, the modified HA protein comprises a substitution of:
(a) isoleucine instead of valine at a position corresponding to position 39 (i.e., V39I);
(b) asparagine instead of lysine at a position corresponding to position 391 (i.e., K391N); and/or
(c) asparagine instead of aspartate at a position corresponding to position 430 (i.e., D430N);
of a full length H2 HA protein.

In some examples, the modified HA protein comprises a modification of one or more amino acid residues at position: (a) V39; (b) K391; (c) D430; (d) V39 and K391; (e) V39 and D430; (f) K391 and D430; or (g) V39, K391 and D430; of a full length H2 HA protein. In other examples, the modified HA protein comprises a modification of: (a) V39I; (b) K391N; (c) D430N; (d) V39I and K391N; (e) V39I and D430N; (f) K391N and D430N; or (g) V39I, K391N and D430N; of a full length H2 HA protein. More particularly, the modified HA protein may comprise, consist of or consist essentially of an amino acid sequence set forth in SEQ ID NO: 60, or a fragment, variant or derivative thereof.

Further contemplated are modified HA proteins having one or more additional amino acid modifications or substitutions at other positions as compared to the respective wild-type HA protein. Thus, modified HA proteins may have at least about 2, 3, 4, 5, 6, 7 or more different residues in other positions as compared to a respective wild-type HA protein. As will be appreciated by those of skill in the art, the number of additional positions that may have amino acid substitutions will depend on the wild-type HA protein or encoding nucleic acid used to generate the variants. To this end, the modified HA proteins provided herein may be derived from any of the known HA sequences from influenza isolates known in the art. For instance, the National Center for Biotechnology information (NCBI) maintains a database (https://www.ncbi.nlm.nih.gov/genomes/FLU/Database/) of known HA sequences. In addition, a database of influenza HA wild-type or MDCK cell and egg-passaged sequences is available from the Global Initiative on Sharing All Influenza Database (GISAID) EpiFlu database.

The modified HA protein may be produced by any means known in the art, including but not limited to, chemical synthesis, recombinant DNA technology, including site-specific mutagenesis, or replacing a portion of the HA coding sequence with a portion that includes the characteristic residue(s), and proteolytic cleavage to produce peptide fragments.

Chemical synthesis is inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al, (John Wiley & Sons, Inc. NY USA 1995-2008). In this regard, reference is also made to International Publication WO 99/02550 and International Publication WO 97/45444.

Recombinant proteins may be conveniently prepared by a person skilled in the art using standard protocols as for example described in Sambrook et al, MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al, (John Wiley & Sons, Inc. NY USA 1995-2008), in particular Chapters 10 and 16; and CURRENT PROTOCOLS IN PROTEIN SCIENCE Eds. Coligan et al, (John Wiley & Sons, Inc. NY USA 1995-2008), in particular Chapters 1, 5 and 6. Typically, recombinant protein preparation includes expression of a nucleic acid encoding the protein in a suitable host cell. Modified HA proteins can, for example, be obtained by mutating the gene or genes (i.e., viral gene segments) encoding the HA protein of interest by site-directed or random mutagenesis. Such mutations may include point mutations, deletion mutations and insertional mutations. For example, one or more point mutations (e.g., substitution of one or more amino acids with one or more different amino acids) may be used to construct the modified HA proteins described herein.

According to particular examples, the modified HA protein has been modified or mutated by one or more passages (e.g., 1, 2, 3, 4, 5, 6, 7 etc passages), such as by serial passage, of an influenza virus isolate expressing an unmodified or wild-type HA protein in cells and/or eggs, such as those provided herein. In some examples, the modified HA protein has been modified or mutated by one or more passages of an influenza virus isolate expressing an unmodified or wild-type HA protein in mammalian cells, such as MDCK cells.

Suitably, the modified HA proteins described herein are immunogenic. As such, the modified HA proteins can be suitable for use as immunogens in a vaccine to treat or prevent an influenza virus infection in human or animals (e.g., avian animals or pigs). As used herein, the term “immunogenic” will be understood to mean that the composition induces or generates an immune response.

In particular examples, the modifications provided herein do not or do not substantially alter or modulate (i.e., increase or decrease) the immunogenicity/antigenicity of the modified HA protein (e.g., relative to or when compared to a wild-type or unmodified version thereof). To this end, it is noted that the trimer interface region is internalised upon HA trimer formation and is typically not accessible by a host's immune system during an influenza virus infection. The immunogenicity or antigenicity of the modified HA protein may be assessed by any means known in the art, such as by determining the presence or amount of a neutralizing antibody or an antibody recognizing the modified HA protein (e.g., in a trimer arrangement) using a standard immunoassay and/or determining predicted or actual T cell reactivity.

It is further envisaged that the modified HA protein may include one or more further modifications as are known in the art. For example, the modified HA protein may be further modified to remove determinants (e.g., hyper-basic regions around the HA1/HA2 cleavage site) that cause a virus to be highly pathogenic. In some examples, the modified HA protein is further modified to remove a polybasic cleavage site therein. This site allows for trypsin independent maturation of HA and typically defines “high pathogenicity” vs “low pathogenicity” avian influenza.

The modified HA protein may also be manipulated so as to constitute a chimeric HA protein (i.e., contain amino acid sequences from more than one influenza strain). For instance and suitably in addition to the one or more modified amino acid residues in the trimer interface region, a chimeric HA may contain the cytoplasmic, or cytoplasmic and transmembrane portions of the HA from one influenza strain and at least the extracellular antigenic portion of the HA from a different influenza strain. This approach has been described previously as a technique for producing influenza viruses containing the antigenic portion of the HA protein in circumstances where the unmodified HA segment may be produced at low yields.

In other examples, the modified HA protein is a non-chimeric HA protein. In other words, the HA sequence comprises the cytoplasmic, transmembrane and extracellular domain from the same influenza strain. In such examples, although the modified HA protein sequence is a non-chimeric sequence, it may comprise other modifications as described herein.

### Encoding Nucleic Acids

The present disclosure also provides an isolated nucleic acid encoding the modified HA protein described herein.

The term “nucleic acid” as used herein designates single- or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as modified purines (for example inosine, methylinosine and methyladenosine) and modified pyrimidines (for example thiouridine and methylcytosine).

It is envisaged that the encoding nucleic acid described herein may directly, such as by way of a viral segment or viral mRNA, or indirectly, such as by way of a viral segment or a complementary DNA sequence that encodes a viral segment, encode the modified HA protein of the present disclosure. In particular examples, the isolated nucleic acid is or comprises a HA viral segment (i.e., an influenza RNA segment) that encodes the modified HA protein provided herein. Suitably, the HA viral segment comprises, consists of or consists essentially of a nucleotide sequence set forth in any one of SEQ ID NOs: 35, 37, 39, 41, 43, 45, 47, 49, 51, 53, 55, 57, 59, 61, 63, 65 or 67, or a fragment, derivative or variant thereof. In some examples, the isolated nucleic is or comprises a nucleotide sequence complementary to a HA viral segment that encodes the modified HA protein provided herein. In alternative examples, the isolated nucleic acid is or comprises a DNA or cDNA sequence that encodes a HA viral segment (i.e., viral RNA) that encodes the modified HA protein provided herein. In various examples, the isolated nucleic acid is or comprises a viral mRNA sequence that encodes the modified HA protein provided herein.

As used herein, a “polynucleotide” is generally a nucleic acid having eighty (80) or more contiguous nucleotides, while an “oligonucleotide” typically has less than eighty (80) contiguous nucleotides. A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “probe” may be a single or double-stranded oligonucleotide or polynucleotide, suitably labelled for the purpose of detecting complementary sequences in Northern or Southern blotting, for example.

Also contemplated herein are fragments, variants and derivatives of the isolated nucleic acid. Variants may comprise a nucleotide sequence at least 70%, at least 75%, preferably at least 80%, at least 85%, more preferably at least 90%, 91%, 93%, 94%, 95%, 96%, 97%, 98% or 99% nucleotide sequence identity with any nucleotide sequence encoding the variant or modified HA protein of the present disclosure (e.g., SEQ ID NOs: 35, 37, 39, 41, 43, 45, 47, 49, 51, 53, 55, 57, 59, 61, 63, 65 or 67). Nucleic acid derivatives may include chemically modified nucleic acids, modified internucleotide linkages, nucleic acid analogues, artificial nucleic acids and combinations thereof, as are known in the art.

Fragments of the isolated nucleic acid may comprise or consist of up to 5%, 10%, 15%, 20%, 25%, 30%, 35%, 40%, 45%, 50%, 55%, 60%, 65%, 70%, 75%, 80%, 85%, 90% or 95-99% of the contiguous nucleotides present in any nucleotide sequence encoding the modified HA protein of the present disclosure, such that they encode at least a portion of the modified HA protein (e.g., encode at least a portion of the trimer interface region of the HA protein). In general, fragments may comprise, consist essentially of or consist of up to 150, 165, 180, 195, 210, 225, 240, 255, 270, 285, 300, 315, 330, 345, 360, 375, 390, 405, 420, 435, 450, 465, 480, 495, 510, 525, 540, 555, 570, 585, 600, 615, 630, 645, 660, 675, 690, 705, 720, 750, 800, 850, 900, 950, 1000, 1050, 1100, 1150, 1200, 1250, 1300, 1350, 1400, 1450, 1500, 1550, 1600, 1650, 1700, 1750, 1800 contiguous nucleic acids that encode a portion of a modified HA protein described herein (e.g., SEQ ID NOs: 35, 37, 39, 41, 43, 45, 47, 49, 51, 53, 55, 57, 59, 61, 63, 65 or 67).

In particular examples, the isolated nucleic acid described herein may be modified to include, or alternatively modified to not include, a 5′ non-coding region (e.g., AGC[A/G]AAAGCAGG (SEQ ID NO: 73), wherein [A/G] indicates a nucleotide variation of A or G at this position) and/or a 3′ non-coding region (e.g., CCTTGTTTCTACT (SEQ ID NO: 74)) of an influenza virus, as are known in the art.

The present disclosure also provides nucleic acids that have been modified such as by taking advantage of codon sequence redundancy. In a more particular example, codon usage may be modified to optimize expression of a nucleic acid in a particular organism or cell type.

The isolated nucleic acids disclosed herein can be conveniently prepared using standard protocols such as those described in Chapter 2 and Chapter 3 of CURRENT PROTOCOLS IN MOLECULAR BIOLOGY (Eds. Ausubel et al. John Wiley & Sons NY, 1995-2008).

Nucleic acids of the present disclosure may be produced, isolated, detected and/or subjected to recombinant DNA technology using nucleic acid sequence amplification techniques.

Suitable nucleic acid amplification techniques covering both thermal and isothermal methods are well known to the skilled addressee, and include polymerase chain reaction (PCR); strand displacement amplification (SDA); rolling circle replication (RCR); nucleic acid sequence-based amplification (NASBA), Q-β replicase amplification, recombinase polymerase amplification (RPA) and helicase-dependent amplification, although without limitation thereto.

### Genetic Constructs

The present disclosure also provides a genetic construct comprising the isolated nucleic acid hereinbefore described. The genetic construct may be a vector.

In particular examples, the genetic construct comprises the isolated nucleic acid operably linked or connected to one or more other genetic components. A genetic construct may be suitable for therapeutic delivery of the isolated nucleic acid (e.g., a DNA or RNA vaccine) or for recombinant production of the modified HA protein of the disclosure in a host cell. Additionally, the genetic construct may be used for the production or generation of influenza viruses (e.g., a variant or modified strain of the influenza virus isolate or a reassortant influenza virus isolate) that expresses the modified HA protein.

Broadly, the genetic construct can be in the form of, or comprises genetic components of, a plasmid, a bacteriophage, a cosmid, a yeast or a bacterial artificial chromosome, as are well understood in the art. Genetic constructs may be suitable for maintenance and propagation of the isolated nucleic acid in bacteria or other host cells, for manipulation by recombinant DNA technology and/or expression of the nucleic acid or an encoded protein of the present disclosure. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like. Such vectors may be or may not be autonomously replicating.

For the purposes of host cell expression, the genetic construct is an expression construct. Suitably, the expression construct comprises the nucleic acid of the present disclosure operably linked to one or more additional sequences in an expression vector. An “expression vector” may be either a self-replicating extra-chromosomal vector, such as a plasmid, or a vector that integrates into a host genome. An expression construct may alternatively be a linear expression construct. Such linear expression constructs will typically not contain any amplification and/or selection sequences. However, linear constructs comprising such amplification and/or selection sequences are also within the scope of the present disclosure. A linear expression construct may, for example, include individual linear expression constructs for each viral segment. It is also possible to include more than one, such as two, three four, five or six, viral segments on the same linear expression construct.

Expression constructs suitable for use in the methods of the present disclosure may be uni-directional or bi-directional expression constructs. As influenza viruses require a protein for infectivity, it is generally preferred to use bi-directional expression constructs as this reduces the total number of expression constructs required by the host cell. Bi-directional expression constructs contain at least two promoters which drive expression in different directions (i.e. both 5′ to 3′ and 3′ to 5′) from the same construct. The two promoters can be operably linked to different strands of the same double stranded DNA. Suitably, one of the promoters is a pol I promoter and at least one of the other promoters is a pol II promoter. Thus, the methods of the present disclosure may utilise at least one bi-directional expression construct wherein at least one gene or cDNA is located between an upstream pol II promoter and a downstream non-endogenous pol I promoter. Transcription of the gene or cDNA from the pol II promoter produces capped positive-sense viral mRNA, which can be translated into a protein, while transcription from the non-endogenous pol I promoter produces negative-sense viral RNA (vRNA).

By “operably linked” is meant that said additional nucleotide sequence(s) is/are positioned relative to the nucleic acid of the present disclosure preferably to initiate, regulate or otherwise control transcription.

Regulatory nucleotide sequences will generally be appropriate for the host cell used for expression. Numerous types of appropriate expression vectors and suitable regulatory sequences are known in the art for a variety of host cells, as described herein. Expression vectors can be designed for expression of the modified HA protein described herein using prokaryotic (e g., E. coli) or eukaryotic cells (e.g., insect cells (using baculovirus expression vectors, see, e.g., Treanor et al., 2007, JAMA, 297(14):1577-1582 incorporated by reference herein in its entirety), yeast cells, plant cells, algae or mammalian cells).

Typically, said one or more regulatory nucleotide sequences may include, but are not limited to, promoter sequences, leader or signal sequences, ribosomal binding sites, polyadenylation sequences, transcriptional start and termination sequences, translational start and termination sequences, and enhancer or activator sequences. Constitutive, repressible or inducible promoters as known in the art are contemplated by the present disclosure.

In some examples, the genetic construct includes one or more untranslated 5′ and/or 3′ regions that are operably linked or connected to a HA viral segment that encodes the modified HA protein. To this end, the UTRs may be derived from the same influenza virus isolate or a different influenza virus isolate from which the modified HA protein is derived.

The expression construct may also include an additional nucleotide sequence encoding a fusion partner (typically provided by the expression vector) so that the recombinant protein is expressed as a fusion protein.

The expression construct may also include an additional nucleotide sequence encoding a selection marker such as amp^{R}, neo^{R} or kan^{R}, although without limitation thereto.

Suitably, the genetic construct provided herein is suitable or adapted for use in the production or generation of reassortant influenza viruses that expresses the modified HA protein by a reverse genetics method or a hybrid reverse genetics-classical reassortment method. Accordingly, the one or more genetic construct(s) provided herein may be introduced into a host cell using any method for introducing expression construct(s) from known reverse genetics techniques.

The genetic constructs provided herein can be introduced into host cells using any technique known to those of skill in the art. For example, genetic constructs can be introduced into host cells by employing electroporation, DEAE-dextran, calcium phosphate precipitation, liposomes, microinjection, or microparticle-bombardment. In some examples, a genetic construct may be in the form of naked nucleic acid. The naked nucleic acid may have been purified from an influenza virus. In another example, a genetic construct may be in the form of transcribed RNA (e.g., viral mRNA). In other examples, a genetic construct may be in the form of one or more shuttle vectors. Examples of shuttle vectors include non-influenza viruses and replicons, for instance alphavirus-based replicons.

The genetic construct provided herein may include an RNA transcription termination sequence. The termination sequence may be an endogenous termination sequence or a termination sequence, which is not endogenous to the host cell. Suitable termination sequences will be evident to those of skill in the art and include, but are not limited to, RNA polymerase I transcription termination sequence, RNA polymerase II transcription termination sequence, and ribozymes. Furthermore, the expression constructs may contain one or more polyadenylation signals for mRNAs, particularly at the end of a gene whose expression is controlled by a pol II promoter.

In another form, the disclosure also provides a plurality of genetic constructs, including a genetic construct that includes a nucleic acid that encodes the modified HA protein described herein and one or more further genetic constructs that may be utilised in preparing reassortant viruses, including 6:1:1 reassortants, 6:2 reassortants and 7:1 reassortants. The further genetic constructs may comprise or encode one or more of NA, PA, PB1, PB2, NP, NS, and M viral segments (i.e., encode one or more of NA, PA, PB1, PB1-F2, PB2, NP, NS1, NEP, M1 and M2 viral proteins).

### Host Cells

The present disclosure also provides a host cell transformed with the isolated nucleic acid and/or the genetic construct described herein.

In a related form, the present disclosure relates to a method of producing the modified HA protein provided herein, said method including the steps of: (i) culturing the previously transformed host cell described herein; and (ii) isolating said modified HA protein from said host cell cultured in step (i).

The host cells may be any as are known in the art. One well known method for influenza virus growth uses specific pathogen-free (SPF) embryonated hen eggs, with virus being inoculated into, grown and purified from the egg contents (i.e., allantoic fluid). Influenza viruses may also be grown in animal cell culture and, for reasons of replication accuracy, speed and patient allergies, this culture method is preferred.

Referring to the cells described herein, the present methods will typically use a cell line, although primary cells may be used as an alternative. Such cells or cell lines may be bacterial, insect cells, yeast cells, plant cells, algae or mammalian cells. Examples of yeast host cells include, but are not limited to S. pombe and S. cerevisiae. Examples of mammalian host cells include, but are not limited to, Crucell Per.C6 cells, Vero cells, CHO cells, VERY cells, BHK cells, HeLa cells, COS cells, MDCK cells, 293 cells, 3T3 cells or WI-38 cells. In certain examples, the hosts cells are myeloma cells, such as NSO cells, 45.6 TG1.7 cells, AF-2 clone 9B5 cells, AF-2 clone 9B5 cells, J558L cells, MOPC 315 cells, MPC-11 cells, NCI-H929 cells, NP cells, NSO/1 cells, P3 NS1 Ag4 cells, P3/NS1/1-Ag4-1 cells, P3U1 cells, P3X63Ag8 cells, P3X63Ag8.653 cells, P3X63Ag8U.1 cells, RPMI 8226 cells, Sp20-Ag14 cells, U266B1 cells, X63AG8.653 cells, Y3.Ag.1.2.3 cells, and YO cells. Non-limiting examples of insect cells include SJ9, SJ21, Trichoplusia ni, Spodoptera frugiperda and Bombyx mori. Exemplary plant cell systems for expression of the modified HA protein are provided in U.S. Pat. Nos. 7,504,560; 6,770,799; 6,551,820; 6,136,320; 6,034,298; 5,914,935; 5,612,487; and 5,484,719, and U.S. patent application publication Nos. 2009/0208477, 2009/0082548, 2009/0053762, 2008/0038232, 2007/0275014 and 2006/0204487

In particular examples, the host cell is mammalian. Suitable mammalian cells include, but are not limited to, hamster, cattle, primate (including humans and monkeys) and dog cells. Various cell types may be used, such as kidney cells, fibroblasts, retinal cells and lung cells, as are known in the art. Examples of suitable hamster cells are the cell lines having the names BHK21 or HKCC. Suitable monkey cells include African green monkey cells, such as kidney cells as in the Vero cell line (Kistner et al. (1998) Vaccine 16:960-8; Kistner et al. (1999) Dev Biol Stand 98:101-110; Bruhl et al. (2000) Vaccine 19:1149-58). Suitable dog cells include canine kidney cells, as in the CLDK and MDCK cell lines (WO97/37000; Brands et al. (1999) Dev Biol Stand 98:93-100; Halperin et al. (2002) Vaccine 20:1240-7; Tree et al. (2001) Vaccine 19:3444-50). Thus, suitable cell lines include, but are not limited to: MDCK; CHO; 293T; BHK; Vero; MRC-5; PER.C6 and WI-38 cell lines.

It is contemplated that the cell or cell line described herein may be suitable for expression of the modified HA protein, such as for the production of a subunit vaccine containing such a protein. According to other examples, the cell or cell line described herein is suitable for growing influenza viruses. Such cell lines may include: MDCK cells derived from Madin Darby canine kidney; Vero cells derived from the African green monkey (Cercopithecus aethiops) kidney; or PER.C6 cells derived from human embryonic retinoblasts (Pau et al. (2001) Vaccine 19:2716-21). These cell lines are widely available, such as from the American Type Cell Culture (ATCC) collection, the Coriell Cell Repositories and the European Collection of Cell Cultures (ECACC). Alternative cell lines may include avian cell lines (see, e.g., WO2003/076601; WO2005/042728; WO2003/043415), including cell lines derived from ducks (e.g., duck retinal cells) or hens (e.g., chicken embryo fibroblasts (CEF)). Examples include avian embryonic stem cells, including the EBx cell line derived from chicken embryonic stem cells, EB45, EB14, EB14-074 and EB66.

Suitably, the cell or cell line are MDCK cells derived from Madin Darby canine kidney. The original MDCK cells are available from the ATCC as CCL-34. Derivatives of MDCK cells may also be used. For instance, the MDCK cell line may be adapted for growth in suspension culture (e.g., ‘MDCK 33016’, deposited as DSM ACC 2219). Similarly, WO2001/064846 discloses a MDCK-derived cell line that grows in suspension in serum-free culture (‘B-702’, deposited as FERM BP-7449). WO2006/071563 discloses non-tumorigenic MDCK cells, including ‘MDCK-S’ (ATCC PTA-6500), ‘MDCK-SF101’ (ATCC PTA-6501), ‘MDCK-SF102’ (ATCC PTA-6502) and ‘MDCK-SF103’ (PTA-6503). WO2005/113758 discloses MDCK cell lines with high susceptibility to infection, including ‘MDCK.5F1’ cells (ATCC CRL-12042). Any MDCK cell line, including those provided herein, can be used in the methods of the present disclosure.

For viral growth or propagation on a cell line, such as on MDCK cells, the influenza virus may be grown on cells in suspension or in adherent culture. Further, the cells described herein can be cultured in various serum-free media or media that is substantially free of serum, as are known to the person skilled in the art (e.g., Iscove's medium, ultra CHO medium (Bio Whittaker), EX-CELL (JRH Biosciences)). Otherwise, the cells for replication can alternatively be cultured in the serum-containing media (e.g., MEM or DMEM medium with about 0.5% to about 10%, more particularly about 1.5% to about 5%, of foetal calf serum) or protein-free media (e.g., PF-CHO (JRH Biosciences)). Suitable culture vessels, which can be employed in the course of the methods described herein can be vessels known to the person skilled in the art, such as, for example, spinner bottles, roller bottles or fermenters.

In particular examples, the cells are suitably grown in serum-free culture media and/or protein free media, such as for cell proliferation and/or supporting influenza virus replication. A medium is referred to as a serum-free medium in the context of the present disclosure if it contains no additives or substantially no additives (e.g., less than 0.5%, 0.25% or 0.1% by weight thereof) from serum of human or animal origin. Protein-free refers to a culture media in which multiplication of the cells occurs with the exclusion of proteins, growth factors, other protein additives and non-serum proteins, but can optionally include proteins such as trypsin or other proteases that may be necessary for viral growth. The cells growing in such cultures naturally contain proteins themselves.

Cell lines supporting influenza virus replication are suitably cultured at a temperature below 37° C. (e.g., about 30° C. to about 36° C., or at about 30° C., 31° C., 32° C., 33° C., 34° C., 35° C., 36° C. or any range therein) during viral replication. Where virus is grown on a cell line then the culture media, and also the viral inoculum used to start the culture, is suitably free from (e.g., will have been tested for and given a negative result for contamination by) contaminating viruses, such as herpes simplex virus, respiratory syncytial virus, parainfluenza virus 3, SARS coronavirus, adenovirus, rhinovirus, reoviruses, polyomaviruses, bimaviruses, circoviruses, and/or parvoviruses.

### Influenza Viruses

In one form, the present disclosure provides an isolated influenza virus, comprising a HA viral segment encoding a modified HA protein, such as that hereinbefore described, comprising an amino acid sequence wherein one or more amino acid residues of a trimer interface region thereof are modified.

Suitably, the HA viral segment has been modified, such as by one or more passages in cells and/or recombinant methods, to encode the modifications to the one or more amino acid residues of the trimer interface region. More particularly, the HA viral segment has suitably been modified by recombinant methods to encode the modified one or more amino acid residues of the modified HA protein prior to incorporation into an isolated influenza virus. In particular examples, the HA viral segment comprises, consists of or consists essentially of a nucleotide sequence selected from the group consisting of SEQ ID NQs: 35, 37, 39, 41, 43, 45, 47, 49, 51, 53, 55, 57, 59, 61, 63, 65 or 67, or a fragment, variant or derivative thereof. In various examples, the HA viral segment encodes the amino acid sequence as set forth in any one of SEQ ID NOs: 36, 38, 40, 42, 44, 46, 48, 50, 52, 54, 56, 58, 60, 62, 64, 66 or 68, or a fragment, variant or derivative thereof.

Influenza viruses are enveloped RNA viruses that belong to the family of Orthomyxoviridae (Palese and Shaw (2007) Orthomyxoviridae: The Viruses and Their Replication, 5th ed. Fields' Virology, edited by B. N. Fields, D. M. Knipe and P. M. Howley. Wolters Kluwer Health/Lippincott Williams & Wilkins, Philadelphia, USA, p 1647-1689). Influenza A and B viruses are major human pathogens, causing a respiratory disease that ranges in severity from sub-clinical infection to primary viral pneumonia which can result in death. The clinical effects of infection vary with the virulence of the influenza strain and the exposure, history, age, and immune status of the host. The natural hosts of influenza viruses are predominantly avian, but influenza viruses, particularly influenza A viruses (including those of avian origin) can also infect and cause illness in humans and other animal hosts (bats, canines, pigs, horses, sea mammals, and mustelids).

The influenza viruses referred to herein encompass any virus type, subtype or strain including, but not limited to, naturally occurring strains, variants or mutants, mutagenized viruses, reassortant viruses and/or genetically modified viruses (e.g., modified by reverse genetics or recombinant DNA technology).

The influenza virus of the present disclosure can be an influenza A virus or an influenza B virus. According to some examples, the influenza virus is an influenza A virus. In alternative examples, the influenza virus is an influenza B virus. The influenza A or B virus may be any strain of virus. By way of examples, the influenza A virus provided herein may include a HA subtype selected from H1, H2, H3, H4, H5, H6, H7, H8, H9, H10, H11, H12, H13, H14, H15 and H16. In particular examples, the influenza A virus is of a H2 subtype (i.e., contains a modified HA protein of a H2 subtype). Moreover, such viruses may contain the influenza A virus NA subtypes N1, N2, N3, N4, N5, N6, N7, N8 or N9. In some examples, the NA protein is of an N1, N2, N3 or N5 subtype. More particularly, the NA protein can be of an N1, N2 or N3 subtype. In certain examples, the influenza A virus can be a strain selected from the group consisting of H1N1, H1N2, H2N1, H2N2, H2N3, H3N1, H3N2, H3N8, H5N1, H7N1, H7N2, H7N3, H7N7, H9N2, and H10N7. According to some examples, the influenza A virus is a H2N1 strain. In alternative examples, the influenza A virus is a H2N2 strain. In other examples, the influenza A virus is a H2N3 strain.

In view of the above, the influenza virus referred to herein is suitably a pandemic influenza virus strain. In this regard, the modified HA protein provided herein may be at least partly derived from a pandemic influenza virus strain. As used herein, the term “pandemic influenza virus strain” refers to a strain of influenza virus being associated or susceptible to be associated with an outbreak of influenza disease. Generally, the characteristics of an influenza strain that give it the potential to cause a pandemic outbreak are: (a) it contains a new haemagglutinin compared to the haemagglutinins in currently-circulating human strains, i.e., one that has not been evident in the human population for over a decade (e.g., H2), or has not previously been seen at all in the human population (e.g., H5, H6 or H9, that have generally been found only in bird populations), such that the human population will be immunologically naïve to the strain's haemagglutinin; (b) it is capable of being transmitted horizontally in the human population; and (c) it is pathogenic to humans. As such, the modified HA protein may be suitable for producing reassortant viruses for use in a vaccine for protecting against potential pandemic virus strains that can or have spread from a non-human animal population to humans. As such, in certain examples, the term pandemic influenza virus strain refers to a strain of influenza A virus. Suitable pandemic strains include, but are not limited to: H5N1, H9N2, H7N7, H2N2, H2N3, H7N1 and H1N1. Others suitable pandemic strains in human are H7N3, H10N7 and H5N2. In one example, the pandemic strain may be an influenza A H1 sub-type other than the (H1N1)pdm09 strain.

Isolated recombinant influenza viruses are contemplated for the present disclosure. A “recombinant” virus is one which has been manipulated in vitro, such as by using recombinant DNA techniques, to introduce changes to the viral genome.

Suitably, the influenza virus is a reassortant virus, such as a recombinant reassortant virus. The term “reassortant virus” denotes a virus which contains genetic material that results from the combination of genetic material of at least two donor viruses (e.g., one or more gene segments from a first parent influenza virus strain (the donor, backbone or seed strain), and one or more gene segments from a second parent influenza virus strain (the vaccine strain)). When the reassortant virus is used for preparing a vaccine composition, its genetic material usually contains at least the HA gene from a seasonal or pandemic influenza virus, whereas the other genes (i.e., backbone genes) are from one or several other donor or seed viruses which have been selected for their ability to grow easily on the substrate of production used for manufacturing the flu vaccine (e.g., the allantoic cavity of embryonated hen's eggs or a permissive cell line) and/or to be less or non-pathogenic to humans. Examples of donor or seed viruses that contribute as donors of backbone genes include A/Puerto Rico/8/1934 (PR8), A/Texas/1/1977, A/New York/55/2004, AJ Ann Arbor/6/60, A/Leningrad/134/17/57, B/Ann Arbor/1/66, B/Florida/4/2006, B/Panama/45/1990 and B/Lee/1940. The reassortant virus may be produced by any method known in the art, inclusive of reverse genetics, classical reassortment and hybrid versions thereof.

Influenza donor strains are strains which typically provide the backbone segment in a reassortant influenza virus, even though they may sometimes also provide the NA segment of the virus. The vaccine strain is the influenza strain that provides the HA and/or NA segment. Generally, both the HA and the NA segment in a reassortant influenza virus will be from the vaccine strain. The vaccine strain is typically a circulating strain, such as a seasonal or pandemic influenza virus strain. Suitably, the vaccine strain is different or heterologous from the donor strain. The genome segments that are present in a reassortant virus can be described using a gene constellation ratio, which indicates the number of segments that are provided by each parent influenza virus strain. For instance, when a reassortant virus contains genome segments from two parent influenza virus strains (such as a donor strain and a vaccine strain), it may have a gene constellation ratio of 1:7, 2:6, 3:5, 4:4, 5:3, 6:2, or 7:1.

Generally, the majority of gene segments of the reassortant viruses are from the donor strain, because it is desirable to harness the properties of the donor strain (e.g., improved replication and/or yield in cell culture) through reassortment of the donor strains segments with segments from the vaccine strain. In particular examples, the reassortant influenza virus produced by the methods provided herein has a gene constellation ratio of 5:3, 6:2, or 7:1, wherein the first number of the ratio indicates the number of segments from the donor strain and the second number of the ratio indicates the number of segments from the vaccine strain.

According to particular examples, the donor strain is a strain that has regulatory approval for use in vaccine manufacture. It is advantageous to use a donor strain that has regulatory approval because reassortant viruses generated by the methods provided herein may be used to prepare vaccines, which may be able to be marketed more easily than if the donor strain does not have pre-existing regulatory approval.

In certain examples, the reassortant influenza virus has a gene constellation ratio of 6:2. For these examples, the reassortant influenza virus comprises six backbone segments (i.e., PB1, PB2, PA, NP, M, and NS) from the donor strain and two segments (i.e., HA and NA) from the vaccine strain. In such examples, the HA viral segment encodes a modified HA protein as provided herein.

In other examples, the reassortant influenza virus has a gene constellation ratio of 7:1. In such examples, the reassortant influenza virus can comprise the six backbone segments from the donor strain, the HA segment from the vaccine strain, and the NA segment from the donor strain. In other words, the reassortant influenza virus comprises the HA segment from the vaccine strain and the remaining seven segments are from the donor strain. In alternative examples, the reassortant influenza virus comprises the six backbone segments and the HA segment from the donor strain, and the NA segment from the vaccine strain. In other words, the reassortant influenza virus comprises the NA segment from the vaccine strain and the remaining seven segments from the donor strain.

In further examples, the reassortant influenza virus has a gene constellation ratio of 5:3. In these examples, the reassortant virus may comprise five backbone segments (i.e. five segments selected from the group consisting of: PB1, PB2, PA, NP, M, and NS) from the donor strain and three segments from the vaccine strain. In such examples, the three segments from the vaccine strain are typically HA, NA and one backbone segment (i.e. one segment selected from the group consisting of: PB1, PB2, PA, NP, M and NS). In particular examples, the three segments from the vaccine strain are HA, NA and PB1 and the remaining five backbone segments (i.e., PB2, PA, NP, M and NS) are from the donor strain.

According to particular examples, the isolated reassortant influenza virus comprises:
(a) one or more PA, PB1, PB2, NP, NS, and M viral segments derived from a first influenza virus isolate (e.g., a donor virus);
(b) a HA viral segment, inclusive of chimeric versions thereof, derived from a second influenza virus isolate (e.g., a vaccine virus), wherein the HA viral segment has been modified to encode a modification to one or more amino acid residues of a trimer interface region thereof; and
(c) optionally an NA viral segment, inclusive of chimeric versions thereof, derived from the first influenza virus isolate, the second influenza virus isolate or a third influenza virus isolate.

To this end, the NA viral segment and the HA viral segment encoding the modified HA protein can be from the same influenza virus isolate. Alternatively, the NA viral segment can be from the same influenza virus isolate (e.g., a donor virus) as the backbone viral segments. Moreover, the NA viral segment can be from an influenza virus isolate that is different to that from which the backbone viral segments and the HA viral segment were derived.

Suitably, the isolated influenza virus provided herein is capable of growth or replication in cells and, more particularly, mammalian cells, such as MDCK cells. To this end, the isolated influenza virus may be capable of forming virions having stabilized or more stable HA trimers (e.g., when compared to an isolated influenza virus having an unmodified HA viral segment that encodes an unmodified HA protein) when cultured in cells. In particular examples, the isolated influenza virus provided herein is capable of enhanced or improved growth or replication in cells, such as MDCK cells, when compared to an isolated influenza virus having an unmodified HA viral segment that encodes an unmodified HA protein.

Accordingly, in another form the present disclosure provides a method of improving the growth of an influenza virus in cells, said method including the step of modifying the influenza virus to express a modified HA protein, such as that described herein, that comprises an amino acid sequence wherein one or more amino acid residues of a trimer interface region thereof is modified.

It is contemplated that the present method may include the further step of performing one or more passages (e.g., 1, 2, 3, 4, 5, 6, 7, 8, 9, 10 etc. passages) of the influenza virus expressing the modified HA protein in cells. As demonstrated in the passage experiments in Example 1, this may assist in inducing further mutations (i.e., in addition to those originally included in the modified HA protein) or combinations of mutations in the trimer interface region of the modified HA protein that are particularly beneficial in facilitating or enhancing growth in influenza viruses and more particularly influenza viruses of a H2 subtype.

Thus, for vaccine viruses that are to be grown or passaged in cells, such as MDCK cells, modification of one or more residues of the trimer interface region (e.g., V39, L219, V233, V320, K383, I388, N390, K391, V392, S394, A405, R416, D430, F450, or any combination thereof, of a HA protein of a H2 subtype), in HA, such as by mutation/substitution, or selection of a HA viral segment that encodes a modified HA protein with a particular amino acid residue modification or substitution (e.g., V39I, L219P, V233A, V320A, V320I, K383E, I388T, N390I, K391R, K391N, V392A, S394Y, A405T, R416G, D430N, F450S or any combination thereof, wherein the numbering is based on full length H2) in HA, may result in increased stability or stabilization of HA trimer formation and/or higher viral titers at end of infection.

In particular examples, the disclosure provides an isolated influenza virus comprising PA, PB1, PB2, NP, NS, M, and NA viral segments (including heterologous or chimeric versions thereof) and a HA viral segment (including heterologous or chimeric versions thereof) that encodes an HA selected to encode one or more modified amino acid residues, such as at V39, L219, V233, V320, K383, I388, N390, K391, V392, S394, A405, R416, D430, F450, or any combination thereof, of H2, wherein the numbering is based on full length H2, wherein the recombinant influenza virus may be capable of growth in cell culture, have enhanced growth/replication in cell culture and/or is capable of forming a HA trimer with enhanced stability, such as during vaccine production. More particularly, the isolated influenza virus may comprise a HA viral segment that encodes an HA protein that includes one or more modified amino acid residues in a lower region of a stalk domain thereof (e.g., one or more of V39, K383, I388, N390, K391, V392, S394, and F450 of a full length H2 HA protein, or any combination thereof, are modified).

As used herein, a “heterologous” influenza virus gene or viral segment is from an influenza virus source or isolate that is different than a majority of the other influenza viral genes or gene segments in a recombinant influenza virus, inclusive of reassortant influenza viruses. Accordingly, a heterologous NA viral segment is suitably derived from an influenza virus source or isolate that is different to that influenza virus source or isolate from which one or more of PA, PB1, PB2, NP, NS and M viral segments are derived. For such examples, the heterologous NA viral segment may be derived from an influenza virus source or isolate that is the same or different to that influenza virus source or isolate from which the HA viral segment (e.g., that encoding the modified HA protein described herein) is derived.

### Methods of Preparing Influenza Viruses

Also provided herein are methods of preparing or producing an influenza virus in cells. Such methods suitably include the step of contacting the cells with a genetic construct comprising a nucleic acid that encodes a modified HA protein, wherein the modified HA protein comprises an amino acid sequence in which one or more amino acid residues of a trimer interface region thereof are modified. It is also envisaged by the present disclosure that methods of classical reassortment (or portions thereof) may be utilised to prepare or produce an influenza virus in cells. By way of example, such methods may include contacting the cells with an influenza virus isolate that expresses a modified HA protein (e.g., the influenza virus isolate comprises a HA viral segment that encodes the modified HA protein), wherein the modified HA protein comprises an amino acid sequence in which one or more amino acid residues of a trimer interface region thereof are modified.

In view of the foregoing, the present methods may be utilised to prepare or produce a modified or variant strain of an influenza virus, such as those provided herein, that includes a modified HA viral segment encoding the modified HA protein described herein. To this end, all of the eight viral segments (i.e., PA, PB1, PB2, NP, NS, M, NA and HA) can be derived from a single influenza virus isolate. In such examples, the influenza virus isolate is suitably a pandemic influenza virus isolate, such as those hereinbefore described. Additionally, such modified or variant strains of an influenza virus are suitably not considered to be a reassortant influenza virus.

Alternatively, the present methods may be utilised to prepare or produce a reassortant influenza virus, such as those provided herein, that includes a HA viral segment encoding the modified HA protein described herein.

Suitably, the present method may be or at least partly comprise a reverse genetics method of generating reassortant viruses. In reverse genetics, the genetic information required to produce the desired influenza virus is delivered to a cell, which is then able to generate influenza virus. Reverse genetics initially required the in vitro assembly and transfection of viral ribonucleoprotein (RNP) into cells infected with a helper virus (Luytjes et al. (1989) Cell 59(6): 1107-1113; Enami et al. (1990) PNAS 87(10):3802-3805). Subsequent techniques involved the transfection of RNA polymerase I plasmids encoding all of the viral RNAs (vRNAs) together with protein expression constructs for the polymerase and NP genes (Fodor et al. (1999) J Virol. 73(11):9679-9682). More recently, reverse genetics methods involve the use of modified RNA polymerase I systems that allow the expression of both negative sense vRNA and positive mRNA from the same template (Hoffmann et al. (2000) PNAS 97(11):6108-6113). In such a method, each of the desired genes is cloned into the pHW2000 plasmid, which consists of the viral cDNA inserted between the RNA polymerase I promoter and termination sequences, and flanked by the CMV promoter and polyadenylation signal. After transfection of the eight plasmids into cells, synthesis of both vRNA and mRNA occurs, resulting in the production of virus. Further refinement has led to the development of systems in which linear DNA expression constructs are used instead of plasmids (WO2009/000891) and the use of a single expression construct (WO2011/012999).

As such, the present method may further include the step of contacting the cells with one or more further genetic constructs, wherein the one or more further genetic constructs comprise one or more further nucleic acids that encode one or more of a PA protein, a PB1 protein, a PB1-F2 protein, a PB2 protein, an NP protein, an NS1 protein, an NEP protein, an M1 protein, an M2 protein and an NA protein. In particular examples, the present method includes one or more of the following steps:
(a) contacting the cells with one or more expression constructs, such as those hereinbefore described, comprising one or more nucleic acid molecules that comprise and/or encode one or more of a PA viral segment, a PB1 viral segment, a PB2 viral segment, a NP viral segment, a M viral segment and a NS viral segment derived from a first influenza virus (e.g., a donor influenza virus);
(b) contacting the cells with one or more expression constructs, such as those hereinbefore described, comprising one or more nucleic acid molecules that comprise or encode a HA viral segment and optionally a NA viral segment derived from a second influenza virus (e.g., a vaccine influenza virus), wherein the HA viral segment encodes the modified HA protein;
(c) culturing the cells in order to produce one or more reassortant viruses; and
(d) selecting for a reassortant virus that comprises the HA viral segment and optionally the NA viral segment from the second influenza virus.

In other examples, the present method may involve a hybrid method of classical reassortment and reverse genetics. For instance, a method in which a host cell is infected with a first influenza strain (e.g., a donor strain) and transfected (e.g., before, after or simultaneously) with one or more expression construct(s) encoding at least one viral segment from a second influenza strain (e.g., HA and optionally NA viral segments from a vaccine strain). An example of such a method is outlined in WO2021099419, which is incorporated by reference herein.

Accordingly, in certain examples, the present method includes one or more of the following steps:
(a) contacting the cells with a donor influenza virus strain comprising a first HA viral segment and a first NA viral segment;
(b) contacting the cells with one or more expression constructs, such as those hereinbefore described, comprising one or more nucleic acid molecules that comprise or encode a second HA viral segment and optionally a second NA viral segment derived from a vaccine influenza virus strain, wherein the second HA viral segment encodes the modified HA protein;
(c) culturing the cells in order to produce one or more reassortant viruses; and
(d) selecting for a reassortant virus that comprises the second HA viral segment and optionally the second NA viral segment.

The term “vaccine influenza virus strain” as used herein refers to an influenza virus strain suitable for use in an immunogenic composition or an immunogenic virus (e.g., a reassortant virus). Vaccine influenza virus strains can include, but are not necessarily limited to, pathogenic strains, non-pathogenic or relatively non-pathogenic strains, killed strains and/or attenuated strains. In particular examples, the vaccine influenza virus strain is a pandemic influenza virus strain.

The vectors or expression constructs utilised in the present method may be as per those known in the art, inclusive of those hereinbefore described. Thus, the present disclosure envisages the use of isolated and purified vectors or plasmids, which express or encode influenza virus proteins, or express or encode influenza vRNA, both native and recombinant vRNA. The vectors may comprise influenza cDNA (see, e.g., Fields Virology (Fields et al. (eds.), Lippincott, Williams and Wickens (2013), which is incorporated by reference herein). Any suitable promoter or transcription termination sequence may be employed to express a protein or peptide, e.g., a viral protein, such as the modified HA protein described herein. By way of the example, one or more of the expression constructs may be adapted for: (a) vRNA production and comprise a promoter operably linked to an influenza virus DNA molecule linked to a transcription termination sequence; and/or (b) mRNA production and comprise a promoter operably linked to a DNA segment encoding an influenza viral segment.

As noted above, an additional selection step to enhance the production of reassortant viruses comprising the modified HA viral segment (i.e., the vaccine strain's HA viral segment that has been modified to encode a modified HA protein) are also contemplated by the present disclosure. The selection step may comprise any method that enhances the selection of reassortant viruses comprising a HA viral segment that encodes a modified HA protein derived from the vaccine strain. Suitably, the selection step is carried out after the host cells have been cultured in order to produce reassortant influenza viruses that may express the modified HA protein.

Suitably, the methods provided herein comprise a step of separating reassortant viruses from the host cells prior to the selection step. By way of example, cell culture supernatant comprising the reassortant viruses is separated from the host cells and the selection step is performed on the supernatant that comprises the reassortant viruses.

In some examples, the selection step comprises negative selection against reassortant viruses comprising the HA protein from the donor strain. Negative selection can include, for example, contacting the host cells, reassortant viruses that have been separated therefrom and/or cell culture supernatant with one or more antibodies that specifically bind or are raised against the HA protein from the donor strain. Negative selection may also comprise exposure of the host cells to inhibitory agents (e.g., short interfering RNAs (siRNA), double-stranded RNAs (dsRNA), micro-RNAs (miRNAs), short hairpin RNAs (shRNA), or small interfering DNAs (siDNAs)) that preferentially or specifically reduce the transcription and/or translation of the donor strain's HA gene or protein relative to the vaccine strain's HA gene or protein. In addition to the above, the selection step may include negative selection against reassortant viruses comprising the NA protein from the donor strain, such as by utilising one or more antibodies that specifically binds or are raised against the NA protein from the donor strain.

In other examples, the selection step is or comprises a positive selection step. The positive selection step may include contacting the host cells, reassortant viruses that have been separated therefrom and/or cell culture supernatant with one or more antibodies that are specific for the modified HA protein derived from the vaccine virus isolate or strain. In this way, reassortant viruses that comprise the modified HA viral segment can be positively selected from the host cells or cell culture supernatant. Suitably, one or more antibodies used for positive selection are labelled (e.g., with a magnetic bead). Labelling aids subsequent isolation, such as by affinity chromatography, of reassortant viruses comprising the HA gene that encodes the modified HA protein.

The methods described herein may comprise one or more positive and/or negative selection steps. For example, reassortant virus may be passaged multiple times in the presence of the one or more antibodies described above for positive and/or negative selection. Multiple selection steps may be performed to enhance the selection of reassortant influenza viruses comprising the HA viral segment that encodes the modified HA protein.

The present methods suitably produce a pool of reassortant viruses from which a particular class of reassortant virus can be isolated. A reassortant virus having, for example, high growth properties and comprising the modified HA protein and optionally NA protein of a seasonal or pandemic influenza strain can be isolated for use in vaccine manufacture. Accordingly, the above methods may further include the step of isolating a reassortant influenza virus comprising the HA viral segment that encodes the modified HA protein.

To this end, the present methods may include harvesting or isolating intact or whole virions from the cell culture media. Alternatively, or additionally, the present methods can include harvesting or isolating split virions from the culture media. In such examples, the harvesting or isolating step suitably includes contacting the influenza virus with a splitting agent, such as a detergent. The present methods may alternatively or additionally include harvesting or isolating one or more particular influenza virus proteins, such as the modified HA protein, from the culture media or from the harvested influenza virus, such as by affinity chromatography.

During harvesting or isolation of the influenza virus and/or the influenza virus proteins, the cells may be separated from the culture medium by standard methods like centrifugation, separation, filtration or ultrafiltration. The influenza virus or the viral proteins produced therefrom can then be concentrated and/or purified according to methods known to those skilled in the art, such as gradient centrifugation (e.g., gradient ultracentrifugation (GUC)), filtration, precipitation, chromatography, and any combination thereof. In particular examples, the influenza virus and/or one or more proteins produced thereby are isolated or harvested from the culture media by gradient ultracentrifugation. Suitably, the influenza viruses are inactivated during or after purification. Virus inactivation can occur, for example, by the contacting the influenza virus with an inactivating agent (e.g., addition of b-propiolactone or formaldehyde) at any point within the purification process.

Suitably, the present method may further include the step of selecting the influenza virus, such as a reassortant influenza virus, that is capable of growth or rescue in cells and, more particularly, mammalian cells like MDCK cells. In particular examples, the present method includes the step of determining whether the influenza virus (e.g., reassortant virus) forms virions that are capable of forming HA trimers (or stable HA trimers) when cultured in cells. Accordingly, in one broad form the present disclosure provides a method of preparing an influenza virus capable of forming stable HA trimers and/or growth in cells.

Based on the foregoing, the present disclosure further provides an isolated influenza virus prepared by the method described herein.

In a related form, the present disclosure also provides a modified HA protein prepared by the method described herein.

### Vaccines

The present disclosure envisages that the influenza virus and/or the modified HA protein derived from the influenza virus produced according to the methods described herein may be utilised in vaccine compositions.

Accordingly, in one form, the present disclosure provides a method of making a vaccine composition, including the steps of:
(a) providing the isolated influenza virus and/or the modified HA protein provided herein; and
(b) combining the isolated influenza virus and/or the modified HA protein with an adjuvant and/or treating the isolated influenza virus with an agent that inactivates or attenuates the virus.

In a related form, the present disclosure relates to a vaccine composition, wherein the vaccine composition is produced according to the methods provided herein.

In another related form, the present disclosure relates to a vaccine composition, wherein the vaccine composition:
(a) comprises the isolated influenza virus described herein and a pharmaceutically acceptable carrier, diluent or excipient; or
(b) comprises the modified HA protein described herein and a pharmaceutically acceptable carrier, diluent or excipient.

Influenza vaccines are generally based either on a live attenuated virus or on an inactivated virus. Inactivated vaccines may be based on whole virions, “split” virions, or on purified surface antigens. Viral antigens can also be presented in the form of virosomes. The present methods can be used for manufacturing any of these types of vaccine. Where an inactivated influenza virus is used, the vaccine may comprise whole virion, split virion, or purified surface antigens (e.g., haemagglutinin and optionally neuraminidase). Chemical means for inactivating a virus include treatment with an effective amount of one or more of the following inactivating agents: detergents, formaldehyde, b-propiolactone, methylene blue, psoralen, carboxyfullerene (C60), binary ethylamine, acetyl ethyleneimine, or combinations thereof. Non-chemical methods of viral inactivation are also known in the art, such as UV light or gamma irradiation. Subunit vaccines that include the modified HA protein described herein are also contemplated.

Virions can be harvested from virus-containing fluids, such as cell culture supernatant, by various methods. For example, a purification process may involve zonal centrifugation using a linear sucrose gradient solution (that optionally includes a detergent to disrupt the virions) or affinity chromatography methods. Antigens may then be purified, after optional dilution, by diafiltration.

The vaccine composition may contain a pharmaceutically-acceptable carrier, diluent or excipient. By “pharmaceutically-acceptable carrier, diluent or excipient” is meant a solid or liquid filler, diluent or encapsulating substance that may be safely used in systemic administration. Depending upon the particular route of administration, a variety of carriers, diluent and excipients well known in the art may be used. These may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates, water and pyrogen-free water.

A useful reference describing acceptable carriers, diluents and excipients is Remington's Pharmaceutical Sciences (Mack Publishing Co. N.J. USA, 1991) which is incorporated herein by reference.

Suitably, for the purposes of eliciting an immune response, certain immunological or immunogenic agents may be used in combination with the immunogenic protein described herein. The term “immunogenic agent” includes within its scope carriers, delivery agents, immunostimulants and/or adjuvants as are well known in the art. As will be understood in the art, immunostimulants and adjuvants refer to or include one or more substances that enhance the immunogenicity and/or efficacy of a composition. Non-limiting examples of suitable immunostimulants and adjuvants include squalane and squalene (or other oils of plant or animal origin), inclusive of squalene oil-in-water emulsions (e.g., MF59, AS03 and AF03); block copolymers; TLR agonists, such as pathogen-derived compounds, including lipopeptides, glycolipids, nucleotides, small-molecule inhibitors and bacterial-derived components, such as flagellin; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); Bordetella pertussis antigens; tetanus toxoid; diphtheria toxoid; surface active substances such as hexadecylamine, octadecylamine, octadecyl amino acid esters, lysolecithin, dimethyldioctadecylammonium bromide, N,N-dicoctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminium phosphate, aluminium hydroxide or alum; interleukins such as interleukin 2 and interleukin 12; monokines such as interleukin 1; tumour necrosis factor; interferons such as gamma interferon; immunostimulatory DNA such as CpG DNA, combinations such as saponin-aluminium hydroxide or Quil-A aluminium hydroxide; saponins, such as Matrix-M; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran alone or with aluminium phosphate; carboxypolymethylene such as Carbopol' EMA; acrylic copolymer emulsions such as Neocryl A640 (e.g. U.S. Pat. No. 5,047,238); water in oil emulsifiers such as Montanide ISA 720; poliovirus, vaccinia or animal poxvirus proteins; or mixtures thereof.

Immunogenic agents may include carriers such as thyroglobulin; albumins such as human serum albumin; toxins, toxoids or any mutant cross-reactive material (CRM) of the toxin from tetanus, diphtheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like.

Alternatively, a fragment or epitope of a carrier protein or other immunogenic protein may be used. For example, a T cell epitope of a bacterial toxin, toxoid or CRM may be used. In this regard, reference may be made to U.S. Pat. No. 5,785,973 which is incorporated herein by reference. It is contemplated that the carrier protein or other immunogenic protein may be directly linked or indirectly linked (such as by way of a linker as are known in the art) to the modified HA protein described herein.

Oil-in-water emulsions have been found to be particularly suitable for use in adjuvanting influenza virus vaccines. Various such emulsions are known, and they typically include at least one oil and at least one surfactant, with the oil(s) and surfactant(s) being biodegradable (metabolisable) and biocompatible. The oil droplets in the emulsion are generally less than 5 μm in diameter, and may even have a sub-micron diameter, with these small sizes being achieved with a microfluidiser to provide stable emulsions. Droplets with an average size less than 220 nm are preferred as they can be subjected to filter sterilization.

In various examples, the oil-in-water emulsion is uniform. A uniform emulsion is characterized in that a majority of droplets (particles) dispersed therein is within a specified size range (e.g., in diameter). A suitable specified size range can be, for example, between about 50-220 nm (e.g., about 50, 60, 70, 80, 90, 100, 110, 120, 130, 140, 150, 160, 170, 180, 190, 200, 210, 220 nm or any range therein), between about 50-180 nm, between about 80-180 nm, between about 100-175 nm, between about 120-185 nm, between about 130-190 nm, between about 135-175 nm, or between about 150-175 nm. In some examples, the uniform emulsion contains <10% of the number of droplets (particles) that are outside of the specified range of diameters. In certain examples, the mean particle size of oil droplets in the oil-in-water emulsion preparation is between about 135-175 nm, e.g., about 155 nm±20 nm as measured by dynamic light scattering, and such a preparation contains not more than 1×10⁷ large particles per mL of the preparation, as measured by optical particle sensing. “Large particles” as used herein mean those having diameters ≥1.2 μm, typically between about 1.2-400 μm. In particular examples, the uniform emulsion contains less than 10%, less than 5%, or less than 3% of the droplets that fall outside of the preferred size range. In some examples, the mean droplet size of particles in an oil-in-water emulsion preparation is between about 125-185 nm, e.g., about 130 nm, about 140 nm, about 150 nm, about 155 nm, about 160 nm, about 170 nm, or about 180 nm, and the oil-in-water emulsion is uniform in that less than 5% of the number of droplets in the preparation fall outside the about 125-185 nm range.

The vaccine composition described herein can be used with oils, such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil obtained from the jojoba bean can also be used. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the vaccine composition described herein. The procedures for separation, purification, saponification and other means necessary for obtaining pure oils from animal sources are well known in the art. Most fish contain metabolizable oils which may be readily recovered. For example, cod liver oil, shark liver oils, and whale oil, such as spermaceti, exemplify several of the fish oils which may be used herein.

A number of branched chain oils are synthesized biochemically in 5-carbon isoprene units and are generally referred to as terpenoids. Shark liver oil contains a branched, unsaturated terpenoid known as squalene, 2,6,10,15,19,23-hexamethyl-2,6,10,14,18,22-tetracosahexaene. Squalane, the saturated analog to squalene, may also be utilised in the present vaccine composition. Fish oils, including squalene and squalane, are readily available from commercial sources or may be obtained by methods known in the art. Other suitable oils are the tocopherols. Mixtures of oils are also envisaged.

Surfactants can be classified by their ‘HLB’ (hydrophile/lipophile balance). Suitably, surfactants described herein have a HLB of at least 10, more particularly at least 15, and even more particularly at least 16. The vaccine composition may include one or more surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol); (octylphenoxy)polyethoxy ethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Exemplary surfactants for including in the emulsion are Tween 80 (polyoxyethylene sorbitan monooleate), Span 85 (sorbitan trioleate), lecithin and Triton X-100.

Mixtures of surfactants can also be used (e.g., Tween 80/Span 85 mixtures). A combination of a polyoxyethylene sorbitan ester, such as polyoxyethylene sorbitan monooleate (Tween 80) and an octoxynol, such as t-octylphenoxypolyethoxyethanol (Triton X-100) is also suitable. Another envisaged combination comprises laureth 9 plus a polyoxyethylene sorbitan ester and/or an octoxynol.

Exemplary amounts of surfactants (% by weight) are: polyoxyethylene sorbitan esters (such as Tween 80) 0.01% to 1%, in particular about 0.1%; octyl- or nonylphenoxy polyoxyethanols (such as Triton X-100, or other detergents in the Triton series) 0.001% to 0.1%, in particular 0.005% to 0.02%; polyoxyethylene ethers (such as laureth 9) 0.1% to 20%, more particularly 0.1% to 10% and even more particularly 0.1% to 1% or about 0.5%.

In particular examples, the oil-in-water emulsions are squalene-in-water emulsions, and more particularly, submicron squalene-in-water emulsions. According to some examples, the vaccine composition comprises MF59.

Any suitable procedure is contemplated for producing vaccine compositions. Exemplary procedures include, for example, those described in New Generation Vaccines (1997, Levine et al., Marcel Dekker, Inc. New York, Basel, Hong Kong), which is incorporated herein by reference.

Any safe route of administration may be employed, including oral, rectal, parenteral, sublingual, buccal, intravenous, intra-articular, intra-muscular, intra-dermal, subcutaneous, inhalational, intranasal, intraocular, intraperitoneal, intracerebroventricular, topical, mucosal and transdermal administration, although without limitation thereto.

Dosage forms include tablets, dispersions, suspensions, injections, solutions, syrups, troches, capsules, nasal sprays, suppositories, aerosols, transdermal patches and the like. These dosage forms may also include injecting or implanting controlled releasing devices designed specifically for this purpose or other forms of implants modified to act additionally in this fashion. Controlled release may be effected by coating with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions may be presented as discrete units, such as capsules, sachets, functional foods/feeds or tablets each containing a pre-determined amount of one or more therapeutic agents of the disclosure, as a powder or granules or as a solution or a suspension in an aqueous liquid, a non-aqueous liquid, an oil-in-water emulsion or a water-in-oil liquid emulsion. Such compositions may be prepared by any of the methods of pharmacy, but all methods include the step of bringing into association one or more agents as described above with the carrier which may constitute one or more necessary ingredients. In general, the compositions are prepared by uniformly and intimately admixing the agents of the disclosure with liquid carriers or finely divided solid carriers or both, and then, if necessary, shaping the product into the desired presentation.

The above compositions may be administered in a manner compatible with the dosage formulation, and in such amount as effective. The dose administered to a subject, in the context of the present disclosure, should be sufficient to affect a beneficial response in a subject over an appropriate period of time (e.g., generate a protective immune response). The quantity of agent(s) to be administered may depend on the subject to be treated inclusive of the age, sex, weight and general health condition thereof, factors that will depend on the judgement of the practitioner.

Also disclosed herein is a container comprising the immunogenic or vaccine compositions disclosed herein. Any suitable container known in the art may be used. For example, the container may be selected from the group consisting of a vial, a syringe, an ampoule, a flask, a fermentor, a bioreactor, a bag, a jar, an ampoule, a cartridge and a disposable pen. In one example, the container is a vial, ampoule or a syringe.

The container may be made of glass, metals (e.g., steel, stainless steel, aluminium, etc.) and/or polymers (e.g., thermoplastics, elastomers, thermoplastic-elastomers). The container may be at least partially siliconized.

The vaccine compositions disclosed herein may further comprise a buffer. The buffer may be any suitable buffer known in the art. For example, the buffer may be a TRIS, acetate, glutamate, lactate, maleate, tartrate, phosphate, citrate, carbonate, glycinate, histidine, glycine, succinate and triethanolamine buffer, and/or phosphate buffer. In one example, the buffer is a phosphate buffer. In another example, the buffer is a succinate buffer. In another example, the buffer is a histidine buffer. In another example, the buffer is a citrate buffer.

The buffer may be selected from USP compatible buffers for parenteral use, in particular, when the pharmaceutical formulation is for parenteral use. For example, the buffer may be selected from the group consisting of: monobasic acids, such as acetic, benzoic, gluconic, glyceric and lactic; dibasic acids, such as aconitic, adipic, ascorbic, carbonic, glutamic, malic, succinic and tartaric; polybasic acids, such as citric and phosphoric; and bases, such as ammonia, diethanolamine, glycine, triethanolamine, and TRIS.

### Methods for Eliciting an Immune Response and Treatment

The influenza virus proteins and the vaccine compositions described herein may be suitable for administration to human or non-human animal subjects, such that the present disclosure provides methods of raising an immune response and/or preventing and/or treating an influenza-associated disease, disorder or condition in a subject. The present disclosure also provides a composition for use as a medicament, and provides the use of a composition of the present disclosure for the manufacture of a medicament for raising an immune response and/or preventing and/or treating an influenza-associated disease, disorder or condition in a subject.

Accordingly, in one form, the present disclosure provides a method of eliciting an immune response in a subject, said method including the step of administering a therapeutically effective amount of the isolated influenza virus, the modified HA protein or the vaccine composition provided herein to the subject to thereby elicit the immune response in the subject.

In a related form, the present disclosure provides a method of preventing and/or treating an influenza-associated disease, disorder or condition in a subject, said method including the step of administering a therapeutically effective amount of the isolated influenza virus, the modified HA protein or the vaccine composition described herein to the subject to thereby prevent and/or treat the influenza-associated disease, disorder or condition.

With respect to the aspects described herein, the term “subject”, “patient” and “individual” includes, but is not limited to, mammals, inclusive of humans, performance animals (such as horses, camels, greyhounds), livestock (such as cows, sheep, horses, pigs, chickens, ducks) and companion animals (such as cats and dogs). Suitably, the subject is a human.

By “elicit an immune response” is meant generate or stimulate the production or activity of one or more elements of the immune system inclusive of the cellular immune system, humoral immune system (i.e., antibodies) and/or the native immune system. Suitably, the immune response described herein includes one or more elements of the immune system, such as T lymphocytes, B lymphocytes, antibodies, neutrophils, dendritic cells inclusive of plasmacytoid dendritic cells, cytokines and/or chemokines. Non-limiting examples of cytokines include pro-inflammatory cytokines such as TNF-α, IL-2, IL-6, IL-8, IL-17A and IL-1 (e.g., IL-1β). A non-limiting example of a chemokine is the neutrophil chemo-attractant IL-8. In certain examples, the immune response that is elicited by the vaccine compositions described herein is protective.

As generally used herein, the terms “immunize”, “vaccinate” and “vaccine” refer to methods and/or compositions that elicit a protective immune response against an influenza virus, whereby subsequent infection by the influenza virus, or a related serotype, strain or variant, is at least partly prevented or minimized.

By “protective immunity” is meant a level of immunity whereby the responsiveness to an antigen or antigens is sufficient to lead to rapid binding and/or elimination of said antigens and thus at least partially ameliorate or prevent a subsequent influenza virus infection in a subject.

By “protective immune response” is meant a level of immune response that is sufficient to prevent or reduce the severity, symptom, aspect, or characteristic of a current and/or influenza virus infection in a subject.

As used herein, the terms “treating”, “treat” or “treatment” refer to a therapeutic intervention that at least partly ameliorates, eliminates or reduces a symptom or pathological sign of an influenza-associated disease, disorder or condition, such as an influenza infection, after it has begun to develop. Treatment need not be absolute to be beneficial to the subject. The beneficial effect can be determined using any methods or standards known to the ordinarily skilled artisan.

As used herein, “preventing”, “prevent” or “prevention” refers to a course of action initiated prior to infection by, or exposure to, an influenza virus or molecular components thereof and/or before the onset of a symptom or pathological sign of the disease, disorder or condition, so as to prevent infection and/or reduce the symptom or pathological sign. It is to be understood that such preventing need not be absolute to be beneficial to a subject. A “prophylactic” treatment is a treatment administered to a subject who does not exhibit signs of the disease, disorder or condition, or exhibits only early signs for the purpose of decreasing the risk of developing a symptom or pathological sign of the disease, disorder or condition.

The vaccines described herein may be used to treat both children and adults. Influenza vaccines are currently recommended for use in paediatric and adult immunisation, from the age of 6 months. Thus, a human subject may be less than 1 year old, 1-5 years old, 5-15 years old, 15-55 years old, or at least 55 years old. Preferred subjects for receiving the vaccines are the elderly (e.g. ≥50 years old, ≥60 years old, and preferably ≥65 years), the young (e.g. ≤5 years old), hospitalised subjects, healthcare workers, armed service and military personnel, pregnant women, the chronically ill, immunodeficient subjects, subjects who have taken an antiviral compound in the 7 days prior to receiving the vaccine, people with egg allergies and people travelling abroad. The vaccines are not suitable solely for these groups, however, and may be used more generally in a population. For pandemic strains, administration to all age groups is preferred.

Treatment can be by a single dose schedule or a multiple dose schedule. Multiple doses may be used in a primary immunisation schedule and/or in a booster immunisation schedule. In a multiple dose schedule the various doses may be given by the same or different routes (e.g., a parenteral prime and mucosal boost, a mucosal prime and parenteral boost). Administration of more than one dose (typically two doses) is particularly useful in immunologically naive patients (e.g., for subjects who have never received an influenza vaccine before), or for vaccinating against a new HA subtype (e.g., in a pandemic outbreak). Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.).

### Screening Methods

The present disclosure further relates to methods that involve testing or screening for modifications in a HA protein, such as that of a H2 subtype that may impart favourable growth characteristics to an influenza virus when grown in cells, such as MDCK cells.

Accordingly, in one form, the present disclosure provides a method of identifying modifications in a HA protein (such as that of a H2 subtype) that facilitate or improve growth of an influenza virus in cells, said method including the steps of:
(a) performing one or more passages of one or more candidate influenza viruses expressing the HA protein in cells;
(b) selecting those candidate influenza viruses that are capable of growth or demonstrate improved growth in cells;
(c) screening the candidate influenza virus selected in step (b) for one or more modifications to the HA protein, such as a trimer region or a lower region of the stalk domain thereof.

Suitably, the candidate influenza viruses are not capable of growth or only capable of limited growth in cells when expressing a wild-type or unmodified version of the HA protein.

In another form, the present disclosure provides a method of identifying or screening modifications in a HA protein (such as that of a H2 subtype) that facilitate or improve growth of an influenza virus in cells, said method including the steps of:
(a) modifying the influenza virus to express a modified HA protein comprising an amino acid sequence wherein one or more amino acid residues of a trimer interface region thereof is modified; and
(b) testing the ability of the modified influenza virus to grow in cells.

Suitably, the present method further includes the step of selecting modifications to the HA protein or the modified HA protein which facilitates or improves growth of the influenza virus in cells. Accordingly, the one or more modifications in the modified HA proteins that are selected based on their ability to facilitate or improve growth of an influenza virus in cell culture may be identified as a result of the present method. Such modifications may then be introduced to a HA protein of a wild-type, recombinant or reassortant virus, such as by those methods described herein, so as to facilitate or improve growth of these influenza viruses in cell culture.

Suitably, the modified HA protein comprises an amino acid sequence wherein one or more amino acid residues of a lower region of a stalk domain of the trimer interface region thereof is modified. As described herein, the lower region of the stalk domain may comprise amino acid residues N26 to D46, L325 to P335, D377 to E397 and L439 to D452 of a full length HA protein of a H2 subtype. In other examples, the lower region of the stalk domain comprises amino acid residues N26 to D46, D377 to E397 and L439 to D452 of a full length HA protein of a H2 subtype.

Referring to some examples, the one or more amino acid residues that are modified are at a position selected from the group consisting of 39, 219, 233, 320, 383, 388, 390, 391, 392, 394, 405, 416, 430, 450, and any combination thereof, of a full length H2 amino acid sequence. More particularly, the one or more amino acid residues that are modified may be selected from the group consisting of V39, L219, V233, V320, K383, I388, N390, K391, V392, S394, A405, R416, D430, F450, and any combination thereof, of a full length H2 amino acid sequence.

In a related form, the present disclosure provides a method of identifying modifications in a HA protein (such as that of a H2 subtype) that facilitate or improve growth of an influenza virus in cells, said method including the steps of:
(a) modifying the influenza virus to express a modified HA protein comprising an amino acid sequence wherein one or more amino acid residues of a trimer interface region thereof is modified; and
(b) performing one or more passages of the modified influenza virus expressing the modified HA protein in cells.

Suitably, the present method further includes the step of screening the modified influenza virus following the one or more passages in cells for one or more further modifications to the modified HA protein. It is envisaged that such screening may be performed by any method, such as DNA and protein sequencing methods, known in the art, including sanger sequencing, chain termination sequencing, dye terminator sequencing, pyrosequencing and mass spectrometry.

According to particular examples of the present method, the modified HA protein is that hereinbefore described.

So that preferred embodiments of the present disclosure may be fully understood and put into practical effect, reference is made to the following non-limiting examples.

### EXAMPLES

### Example 1

The aim of the present Example was to rescue a synthetic seed virus based on an influenza A H2N3 pre-pandemic strains A/Chicken/ohio/494832/2007 and A/Swine/Missouri/2124514/2006.

### Methods & Results

The present inventors found, however, that the vast majority of attempts to rescue a synthetic virus using the WT H2N3 alleles from A/Chicken/Ohio/494832/2007 failed to produce a virus. Those which did, produced viruses with mutations or variants in the HA viral segment. All variants were identified by visual analysis of Sanger sequencing results. Variants were identified if multiple peaks were observed at a given nucleotide base in all reads with a minimum read depth of 2 reads. Only variants which resulted in an amino acid change were studied further in this Example.

The HA mutations/variants were identified by one of 4 distinct pathways
1. Passaging of WT A/Chicken/Ohio/494832/2007. Sequencing of this virus has revealed 4 identifiable variants (lx)
   a. A/chicken/ohio/484832/2007
      i. V129I variant—This variant is not at or near the trimer interface. It is on the outside of the head domain and is unlikely to contribute to trimer stability.
      ii. V320I variant
      iii. N390I variant
      iv. K391R variant
2. Rescue of A/Swine/Missouri/2124514/2006 (2× independent rescues).
   a. RG4 A/swine/Missouri/2124514/2006
      i. K383E variant
   b. RG5 A/swine/Missouri/2124514/2006
      i. L219P mutation
3. Rescue of A/Chicken/Ohio/494832/2007
   a. GDE 80.4A A/chicken/ohio/484832/2007
      i. V233A variant
      ii. V320A variant
   b. GDE 80.4B A/chicken/ohio/484832/2007
      i. V39I mutation
   c. GDE 80.7B A/chicken/ohio/484832/2007
      i. F450S
   d. HS_Sys_14 A/chicken/ohio/484832/2007
      i. S394Y variant
      ii. R416G variant
   e. HS_Sys_15 A/chicken/ohio/484832/2007
      i. A405T variant

### Synthetic Seed Process & Procedure

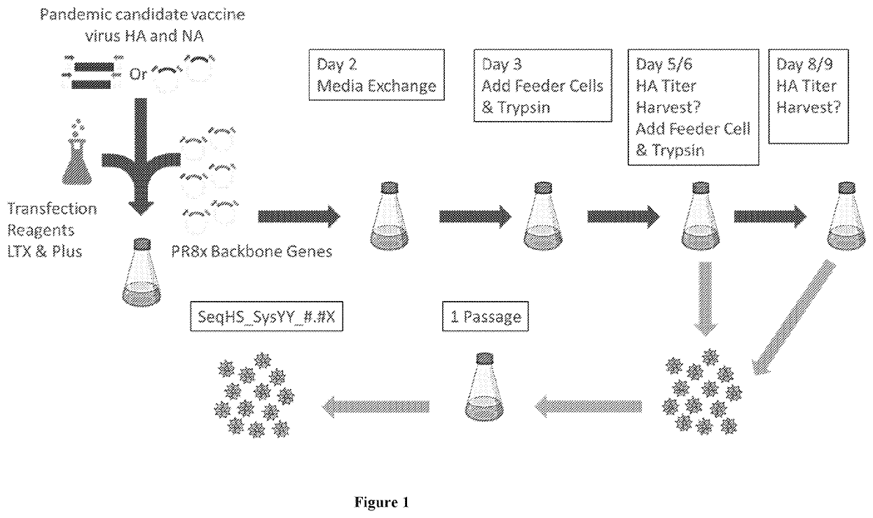

Recombinant assembly of HA and NA sequences of A/Chicken/Ohio/494832/2007 by method described in Dormitzer et al. (Sci Transl Med. 2013 May 15; 5(185)) (see, e.g., FIG. 1 of Dormitzer et al.) into expression constructs.
MDCK cells are then transfected with expression constructs for the HA and NA sequences of A/Chicken/Ohio/494832/2007 and the backbone viral segments for PA, PB1, PB2, NP, NS, and M from a high growth parent strain (e.g., A/Puerto Rico/8/1934 or a cell-adapted version thereof, such as PR8X)
Reassortant viruses are then rescued and characterized
A schematic diagram of a transfection and rescue procedure is provided in FIG. 1.
Two initial rescue attempts failed to generate any rescued isolates of the H2N3 strain.
Amplification of viral RNA from the supernatant of transfected cells confirmed that there was no amplification of viral RNA.
Additional transfection and rescue experiments were performed with 5 out of a total of 28 experiments yielding a rescued viral isolate.

### Sequence Analysis

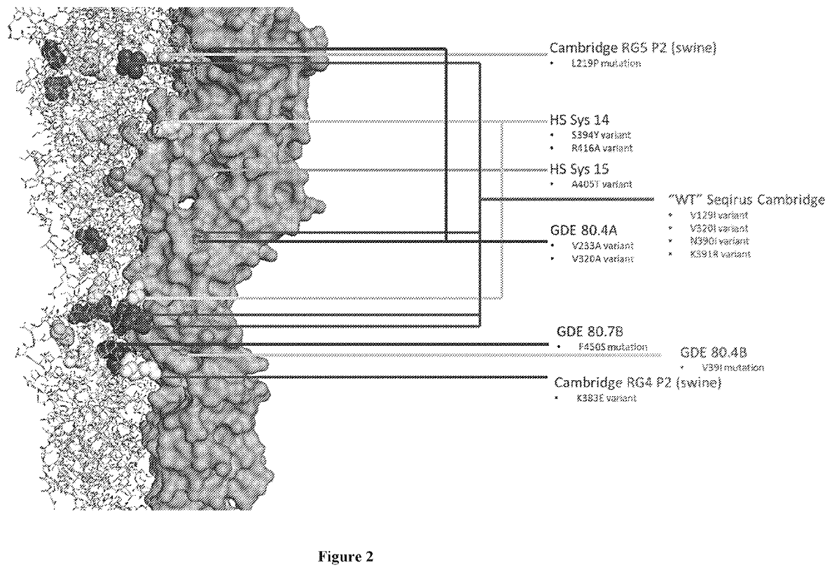

Upon sequencing of the 8 rescued or serially passaged isolates of the H2N3 strains, all were shown to contain variant nucleotide bases in the HA viral segment than encode for modified amino acid residues in the HA protein (see FIG. 2).
No other modifications were observed in any of the other 7 viral segments for these strains.

### Mutation Mapping

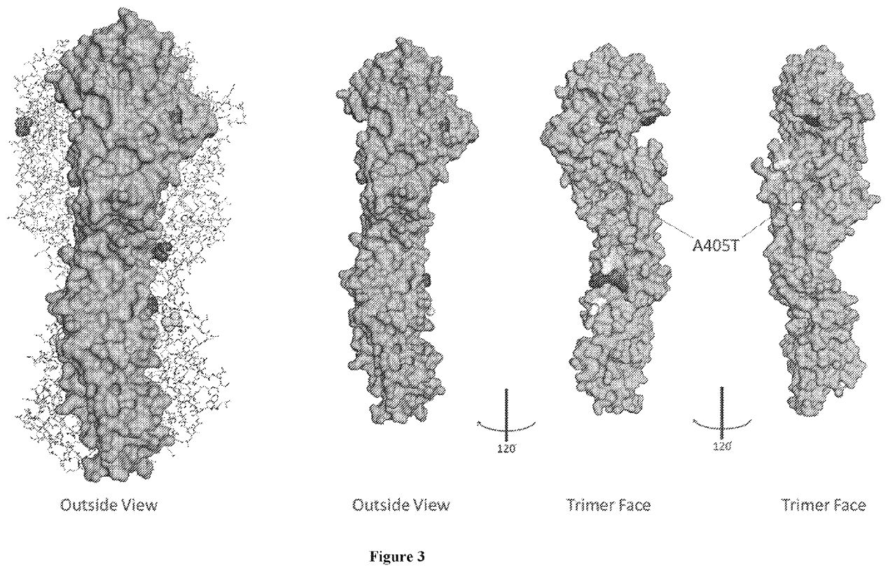

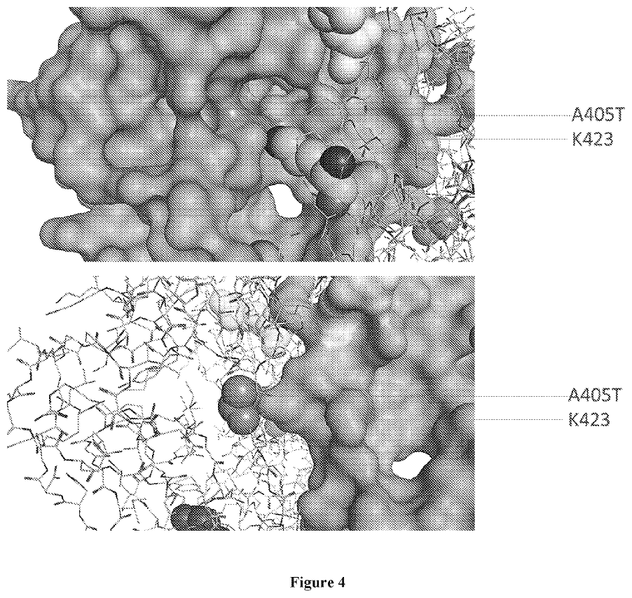

The structure of the A/Swine/Missouri/2124514/2006 HA protein has been resolved.
When the variants identified in these rescues are mapped to that structure, they all exist at the interface between adjacent monomers in the HA trimer structure, as can be observed in FIG. 3.
FIG. 4, further demonstrates that the A405T mutation in the HS Sys 15 isolate brings this residue in closer association with the K423 residue on an adjacent HA monomer when in a homotrimeric arrangement which may function to stabilize the trimer structure.
Without being bound by any theory and based on the location of the variants, and the fact that the present inventors were unable to rescue the WT virus in the absence of at least one variant position, it is hypothesized that the wild-type H2 monomer cannot form functional trimers when grown in cells, which thereby prevents or limits viruses expressing this HA from being propagated in cell culture.

### Clonal Isolation

Clonal isolation experiments were performed to determine the stability of the A405T mutation in the HS Sys 15 isolate.
Several 10 fold limiting dilutions were performed:
   10× plates from −4 to −9 serial dilutions for a total of 60 plates
   Perform titer check on day 3
   Ideally the fewer colonies a plate has, the more likely it is to be clonal.
   The more rounds of clonal isolation, the more likely your isolate is to be clonal
28 clones were isolated from the 60 plates.
   12 clones were from the −5 dilution plate
   16 clones were from the −4 dilution plate
Sequencing was performed on all 28 clones, with the vast majority (25 out of 28) demonstrating a clean population of virus with the A405T mutation for each of the clones, whilst the remaining 3 clones demonstrated evidence of a mixture of the A405T mutant virus and WT virus with the WT virus being only a minor variant thereof.
This data demonstrates that the A405T mutation in the HS Sys 15 isolate is stable.
Similar to the V129I variant, the E184K variant is not at or near the trimer interface. Again, it is on the outside of the head domain and is unlikely to contribute to trimer stability.

### Further Passaging

The present inventors took five of the H2N3 isolates and performed an additional 3 passages (in duplicate) to determine the fidelity of these variants. Sanger sequencing of this passaged material showed additional variants, as outlined in Table 1.
In HS_Sys_15, the A405T variant became the dominant allele and did not pick up additional mutations.
Both variants found in HS_Sys_14 were stable through passaging and did not pick up additional mutations.
Many of the additional variants which appeared, were present more than once, and, as such, there appears to be some convergence towards the HA viral segments acquiring specific mutations.
Residues 39, 383, 388, 390, 391, 392, 394, and 450 all map to a lower region of the stalk (i.e., a lower stalk cluster).

TABLE 1
List of identified variants in rescue viruses after 3 passages.

| | | Passage 3 | Passage 3 |
|---|---|---|---|
| | Rescue | Replicate A | Replicate B |
| GDE-80.4A | V233A variant | X | V233A variant |
| | V320A variant | X | X |
| | | [symbol] | [symbol] |
| | | K391N variant | K391N variant |
| GDE-80.4B | V39I mutation | V39I mutation | V39I mutation |
| | | D430N variant | I388T variant |
| | | E184K variant | V392A variant |
| GDE-80.7B | [symbol] | X | X |
| | | D430N variant | X |
| | | K391N variant | X |
| | | V39I variant | V39I mutation |
| Sys 14 | S394Y variant | S394Y variant | S394Y variant |
| | [symbol] | [symbol] | [symbol] |
| Sys 15 | A405T variant | A405T mutation | A405T mutation |

[symbol]

indicates data missing or illegible when filed

### CONCLUSIONS

The present Example identified a number of mutations which can support rescue or growth of a H2 influenza virus on MDCK cells. These mutations appear along the length of the molecule and consistently at the trimer interface region between HA monomers. A specific region in the lower stalk appears to be a hot spot for mutations which supports viral rescue, but variations were also identified in other locations on the HA molecule. The passaging data suggests that some mutations are more stable over time than others, but minimally the present Example suggests that all of these mutations on the trimer interface at least contribute to viability of the virus when grown in cell culture. To this end, the present Example suggests that the wild-type or unmodified H2 HA protein is close to being able to support growth of H2 influenza viruses in cells. Moreover, the above data supports the notion that a dynamic range of mutations throughout the trimer interface region can promote rescue and growth of H2 influenza viruses in cell culture and that viruses can further switch and alter these modifications with passaging to further support their stability or growth in cells.

### Itemized Listing of Embodiments

1. A modified haemagglutinin (HA) protein comprising an amino acid sequence wherein one or more amino acid residues of a trimer interface region thereof is modified, wherein an influenza virus expressing the modified HA protein is capable of growth in cells.
2. The modified HA protein of Claim 1, which is capable of forming a HA trimer when expressed by an influenza virus grown in cells.
3. The modified HA protein of Claim 1 or Claim 2, wherein the modifications to the one or more amino acid residues of the trimer interface region increases the stability of a HA trimer formed from the modified HA protein relative to a HA trimer formed from a corresponding unmodified HA protein.
4. The modified HA protein of any one of the preceding claims, wherein the modified HA protein is of a H2, H1, H5, H3, H7 or H9 subtype.
5. The modified HA protein of Claim 4, wherein the modified HA protein is of a H2, H1, or H5 subtype.
6. The modified HA protein of Claim 4 or Claim 5, wherein the modified HA protein is of a H2 subtype.
7. The modified HA protein of Claim 6, wherein the one or more amino acid residues that are modified are selected from the group consisting of V39, L219, V233, V320, K383, I388, N390, K391, V392, S394, A405, R416, D430, F450, and any combination thereof, of a full length H2 amino acid sequence.
8. The modified HA protein of Claim 7, wherein the one or more amino acid residues that are modified comprise:
   (a) V39;
   (b) L219;
   (c) V233;
   (d) V320;
   (e) K383;
   (f) I388;
   (g) N390;
   (h) K391;
   (i) V392;
   (j) S394;
   (k) A405;
   (l) R416;
   (m) D430;
   (n) F450;
   (o) S394 and R416;
   (p) V233 and V320;
   (q) V320, N390 and K391;
   (r) V320 and N390;
   (s) V320 and K391;
   (t) N390 and K391;
   (u) F450 and K391;
   (v) V233, F450 and K391;
   (w) V233 and F450;
   (x) V233 and K391;
   (y) V39 and D430;
   (z) V39, I388 and V392;
   (aa) V39 and I388;
   (ab) V39 and V392;
   (ac) I388 and V392;
   (ad) V39, K391 and D430;
   (ae) V39 and K391; or
   (af) K391 and D430;
of a full length H2 amino acid sequence.
9. The modified HA protein of Claim 7 or Claim 8, wherein the modifications to the one or more amino acid residues are selected from the group consisting of V39I, L219P, V233A, V320A, V320I, K383E, I388T, N390I, K391R, K391N, V392A, S394Y, A405T, R416G, D430N, F450S and any combination thereof, of a full length H2 amino acid sequence.
10. The modified HA protein of Claim 9, wherein the modifications to the one or more amino acid residues comprise:
   (a) V39I;
   (b) L219P;
   (c) V233A;
   (d) V320A;
   (e) V320I;
   (f) K383E;
   (g) I388T;
   (h) N390I;
   (i) K391R;
   (j) K391N;
   (k) V392A;
   (l) S394Y;
   (m) A405T;
   (n) R416G;
   (o) D430N;
   (p) F450S;
   (q) S394Y and R416G;
   (r) V233A and V320A;
   (s) V320I, N390I and K391R;
   (t) V320I and N390I;
   (u) V320I and K391R;
   (v) N390I and K391R;
   (w) F450S and K391N;
   (x) V233A, F450S and K391N;
   (y) V233A and F450S;
   (z) V233A and K391N;
   (aa) V39I and D430N;
   (ab) V39I, I388T and V392A;
   (ac) V39I and I388T;
   (ad) V39I and V392A;
   (ae) I388T and V392A;
   (af) V39I, K391N and D430N;
   (ag) V39I and K391N; or
   (ah) K391N and D430N;
of a full length H2 amino acid sequence.
11. The modified HA protein of any one of the preceding claims, wherein one or more of the modified amino acid residues are present in a lower region of the stalk domain.
12. The modified HA protein of Claim 11, wherein the lower region of the stalk domain comprises, consists of or consists essentially of amino acid residues at positions 377 to 397, 439 to 452, 26 to 46 and optionally 325 to 335 of a full length HA protein of a H2 subtype.
13. The modified HA protein of Claim 11, wherein the lower region of the stalk domain comprises, consists of or consists essentially of amino acid residues at positions 383 to 394, 439 to 452, 31 to 40 and optionally 325 to 335 of a full length HA protein of a H2 subtype.
14. The modified HA protein of Claim 11, wherein the lower region of the stalk domain comprises, consists of or consists essentially of amino acid residues N26 to D46, L325 to P335, D377 to E397 and L439 to D452 of a full length HA protein of a H2 subtype.
15. The modified HA protein of Claim 14, wherein the lower region of the stalk domain comprises amino acid residues N26 to D46, D377 to E397 and L439 to D452 of a full length HA protein of a H2 subtype.
16. The modified HA protein of any one of Claims 11 to 15, wherein the one or more amino acid residues that are modified are selected from the group consisting of V39, K383, I388, N390, K391, V392, S394, F450, and any combination thereof, of a full length H2 amino acid sequence.
17. The modified HA protein of any one of Claims 11 to 16, wherein the modifications to the one or more amino acid residues are selected from the group consisting of V39I, K383E, I388T, N390I, K391R, K391N, V392A, S394Y, F450S and any combination thereof, of a full length H2 amino acid sequence.
18. The modified HA protein of any one of the preceding claims, comprising, consisting of or consisting essentially of the amino acid sequence as set forth in any one of SEQ ID NOs: 36, 38, 40, 42, 44, 46, 48, 50, 52, 54, 56, 58, 60, 62, 64, 66 or 68, or a fragment, variant or derivative thereof.
19. The modified HA protein of any one of the preceding claims, wherein the modified HA protein has been modified by: (a) one or more passages of an influenza virus isolate in cells and/or eggs; and/or (b) recombinant methods.
20. A modified HA protein of a H2 sub-type comprising an amino acid sequence wherein one or more amino acid residues thereof is modified at a position selected from the group consisting of V39, L219, V233, V320, K383, I388, N390, K391, V392, S394, A405, R416, D430, F450, and any combination thereof, of a full length H2 amino acid sequence.
21. The modified HA protein of Claim 20, wherein an influenza virus expressing the modified HA protein is capable of growth in cells.
22. The modified HA protein of Claim 21, wherein the cells are MDCK cells.
23. A modified HA protein of a H2 sub-type comprising an amino acid sequence wherein one or more amino acid residues thereof is modified in a trimer interface region, and wherein an influenza virus expressing the modified HA protein is capable of growth in cells.
24. An isolated nucleic acid comprising a nucleotide sequence encoding the modified HA protein of any one of Claims 1 to 23 or a nucleotide sequence complementary thereto.
25. The isolated nucleic acid of Claim 24, comprising, consisting of or consisting essentially of: a nucleotide sequence selected from the group consisting of SEQ ID NQs: 35, 37, 39, 41, 43, 45, 47, 49, 51, 53, 55, 57, 59, 61, 63, 65 or 67, or a fragment, variant or derivative thereof; or a nucleotide sequence complementary thereto.
26. A genetic construct comprising: (i) the isolated nucleic acid of Claim 24 or Claim 25; or (ii) a nucleotide sequence complementary thereto; operably linked or connected to one or more regulatory sequences.
27. A host cell transformed with the isolated nucleic acid according to Claim 24 or Claim 25 or the genetic construct of Claim 26.
28. A method of producing the modified HA protein of any one of Claims 1 to 23, said method including the steps of: (i) culturing the previously transformed host cell of Claim 27; and (ii) isolating said modified HA protein from said host cell cultured in step (i).
29. An isolated influenza virus, comprising a HA viral segment encoding a modified HA protein comprising an amino acid sequence wherein one or more amino acid residues of a trimer interface region thereof are modified.
30. The isolated influenza virus of Claim 29, wherein the modified HA protein is that of any one of Claims 1 to 23.
31. The isolated influenza virus of Claim 29 or Claim 30, wherein the HA viral segment comprises, consists of or consists essentially of a nucleotide sequence selected from the group consisting of SEQ ID NQs: 35, 37, 39, 41, 43, 45, 47, 49, 51, 53, 55, 57, 59, 61, 63, 65 or 67, or a fragment, variant or derivative thereof.
32. The isolated influenza virus of any one of Claims 29 to 31, which is a recombinant influenza virus.
33. The isolated influenza virus of any one of Claims 29 to 32, which is a reassortant influenza virus.
34. The isolated influenza virus of any one of Claims 29 to 33, which is capable of growth in cells.
35. The isolated influenza virus of Claim 34, which is capable of growth in MDCK cells.
36. The isolated influenza virus of any one of Claims 29 to 35, which is capable of forming HA trimers that include the modified HA protein when grown in cells.
37. The isolated influenza virus of any one of Claims 29 to 36, further comprising:
   (a) one or more of PA, PB1, PB2, NP, NS, and M viral segments from a donor influenza virus; and
   (b) a heterologous or chimeric NA viral segment.
38. A method of preparing an influenza virus in cells, said method including the step of contacting the cells with a genetic construct comprising a nucleic acid that encodes a modified HA protein, wherein the modified HA protein comprises an amino acid sequence in which one or more amino acid residues of a trimer interface region thereof are modified.
39. The method of Claim 38, wherein the modified HA protein is that of any one of Claims 1 to 23, the nucleic acid is the isolated nucleic acid of Claim 24 or Claim 25 and/or the genetic construct is that of Claim 26.
40. The method of Claim 38 or Claim 39, further including the step of contacting the cells with one or more further genetic constructs, wherein the one or more further genetic constructs comprise one or more further nucleic acids that encode one or more of a PA protein, a PB1 protein, a PB1-F2 protein, a PB2 protein, an NP protein, an NS1 protein, an NEP protein, an M1 protein, an M2 protein and an NA protein.
41. The method of Claim 40, wherein the NA protein is of an N1, N2 or N3 subtype.
42. The method of any one of Claims 38 to 41, in which a modified pandemic influenza virus is prepared that comprises the nucleic acid that encodes the modified HA protein.
43. The method of any one of Claims 38 to 42, further including the step of isolating or harvesting the influenza virus and/or the modified HA protein from the cells.
44. An isolated influenza virus prepared by the method of any one of Claims 38 to 43.
45. A modified HA protein prepared by the method of any one of Claim 28 or 38 to 43.
46. A method of making a vaccine composition, including the steps of:
   (a) providing the isolated influenza virus of any one of Claims 29 to 37 or 44 and/or the modified HA protein of any one of Claims 1 to 23 or 45; and
   (b) combining the isolated influenza virus and/or the modified HA protein with an adjuvant and/or treating the isolated influenza virus with an agent that inactivates or attenuates the virus.
47. The method of claim 46, wherein the adjuvant comprises an immunostimulatory DNA sequence, a bacterium-derived component, an aluminium salt (alum) or a squalene oil-in-water emulsion system.
48. A vaccine composition, wherein the vaccine composition is produced according to the method of Claim 46 or Claim 47.
49. A vaccine composition, wherein the vaccine composition comprises:
   (a) the isolated influenza virus of any one of Claims 29 to 37 or 44 and a pharmaceutically acceptable carrier, diluent or excipient; or
   (b) the modified HA protein of any one of Claims 1 to 23 or 45 and a pharmaceutically acceptable carrier, diluent or excipient.
50. A method of eliciting an immune response in a subject, said method including the step of administering a therapeutically effective amount of the isolated influenza virus of any one of Claims 29 to 37 or 44, the modified HA protein of any one of Claims 1 to 23 or 45 or the vaccine composition of Claim 48 or Claim 49 to the subject to thereby elicit the immune response in the subject.
51. A method of preventing and/or treating an influenza-associated disease, disorder or condition in a subject, said method including the step of administering a therapeutically effective amount of the isolated influenza virus of any one of Claims 29 to 37 or 44, the modified HA protein of any one of Claims 1 to 23 or 45 or the vaccine composition of Claim 48 or Claim 49 to the subject to thereby prevent and/or treat the influenza-associated disease, disorder or condition.
52. A method of identifying or screening for modifications in a HA protein that facilitate or improve growth of an influenza virus in cells, said method including the steps of:
   (a) modifying the influenza virus to express a modified HA protein comprising an amino acid sequence wherein one or more amino acid residues of a trimer interface region thereof is modified; and
   (b) testing the ability of the modified influenza virus to grow in cells.
53. The method of Claim 52, further including the step of selecting the modified HA protein which facilitates or improves growth of the influenza virus in cells.
54. The method of Claim 52 or Claim 53, wherein the modified HA protein comprises an amino acid sequence wherein one or more amino acid residues of a lower stalk region of the trimer interface region thereof is modified.
55. A method of identifying modifications in a HA protein that facilitate or improve growth of an influenza virus in cells, said method including the steps of:
   (a) modifying the influenza virus to express a modified HA protein comprising an amino acid sequence wherein one or more amino acid residues of a trimer interface region thereof is modified; and
   (b) performing one or more passages of the modified influenza virus expressing the modified HA protein in cells.
56. The method of Claim 55, further including the step of screening the modified influenza virus following the one or more passages in cells for one or more further modifications to the modified HA protein.
57. A method of improving the growth of an influenza virus in cells, said method including the step of modifying the influenza virus to express a modified HA protein comprising an amino acid sequence wherein one or more amino acid residues of a trimer interface region thereof is modified.
58. The method of Claim 57, further including the step of performing one or more passages of the influenza virus expressing the modified HA protein in cells.
59. The method of any one of Claims 55 to 58, wherein the modified HA protein is that according to any one of Claims 1 to 23.
60. A method of improving the stability of an influenza virus strain in cells, said method including the step of modifying the influenza virus strain to express a modified HA protein comprising an amino acid sequence wherein one or more amino acid residues of a trimer interface region thereof is modified.
61. The method of Claim 60, further including the step of performing one or more passages of the influenza virus expressing the modified HA protein in cells.
62. The method of any one of Claims 60-61, wherein the modified HA protein is that according to any one of Claims 1 to 23.
63. A method of improving manufacture of an influenza virus strain, said method including the step of modifying the influenza virus strain to express a modified HA protein comprising an amino acid sequence wherein one or more amino acid residues of a trimer interface region thereof is modified.
64. The method of Claim 63, wherein the influenza virus strain is not capable of growth in culture when expressing a wild-type or unmodified HA protein.
65. The method of Claim 63 or 64, wherein manufacture of the influenza virus strain occurs in cells.
66. The method of any one Claims 60-62 or 65, wherein the cells are selected from the group consisting of mammalian, avian, yeast, and plant.
67. The method of Claim 66, wherein the cells are mammalian cells.
68. The method of Claim 67, wherein the cells are canine kidney cells.
69. The method of Claim 68, wherein the canine kidney cells are Madin Darby Canine Kidney (MDCK) cells.
70. The method of any one of claims 60-69, wherein the influenza virus strain is characterized by at least one of the following: (a) expresses a HA protein that is not present in currently-circulating human strains, or has not previously been detected in a human population, such that the human population will be immunologically naïve to the influenza virus strain's HA protein; (b) capable of being transmitted horizontally in the human population; and (c) pathogenic to humans.

Sequence Listing

| SEQ ID NO: 1 | Nucleotide sequence of PB2 viral segment of A/Chicken/Ohio/494832/2007 |
|---|---|
| | gactaagagatctaatgtcacagtctcgcactcgcgagatactcaccaaaaccactgtggaccacatggccataatcaaa |
| | aagtacacatcaggaaggcaagagaagaaccctgcactcaggatgaagtggatgatggcaatgaaatatccaattacagc |
| | agacaagagaataatggaaatgattcctgaaagaaatgaacaggggcaaaccctttggagcaaaacaaatgatgccggct |
| | cagaccgagtgatggtatcacctctggctgtgacatggtggaataggaatggaccaacaactagtacagttcattatcca |
| | aaggtctataaaacttatttcgaaaaagtcaaaaggttgaaacacgggacctttggccctgttcacttcagaaatcaagt |
| | taaaataagacggagggttgacataaaccctggtcatgcagacctcagtgccaaagaggcacaggatgtaatcatggaag |
| | tcgttttcccaaatgaagtgggagcgagaatactaacatcggagtcacaactgacgataacaagagagaagaaagaagaa |
| | ctccaggactgcaagatcgcccctttgatggttgcgtacatgctagaaagggagttggttcgtaaaacgaggttcctccc |
| | agtggccggtggaacaagcagtgtttacattgaggtgctgcatttaactcaggggacatgctgggagcaaatgtacaccc |
| | caggaggggaagtgaggaatgatgatgttgaccaaagcttgattatcgctgccaggaacatagtaagaagggcaacagta |
| | tcagcagacccactagcatctctattggagatgtgccacagcacacagattggagggataaggatggtagacattcttcg |
| | gcaaaatccaacagaggaacaagctgtggacatatgcaaagcagcaatgggcttaaggattagctcatctttcagctttg |
| | gcggattcactttcaaaagaacaagcgggtcgtcagttaagaaagaagaggaagtgcttacaggcaaccttcaaacatta |
| | aaaataagagtacatgaggggtatgaagagttcacaatggttgggagaagagcaacagccattctcaggaaggcaaccag |
| | aagattgatccagttaatagtaagtgggagagacgagcagtcaattgctgaagcaataattgtggccatggtattttcac |
| | aagaggattgcatgatcaaagcagttcgaggtgatttgaactttgtcaatagggcaaaccagcggctaaatccaatgcat |
| | caactcttgagacacttccaaaaagatgcaaaagtgcttttccaaaactggggaattgaacccattgacaatgtaatggg |
| | aatgatcgggatactacctgacatgaccccaagtactgagatgtcgctaagagggataagagtcagtaagatgggagtag |
| | atgaatactccagcacagagagagtggtagtgagcattgaccgatttttaagagtccgggaccaacgggggaatgtgcta |
| | ttgtcacctgaagaagtcagcgagacacaagggacagaaaagctgacaataacttattcgtcatcaatgatgtgggaaat |
| | caatggtcctgaatcggttttggtcaatacgtatcagtggatcatcagaaattgggaaactgtgaaaattcaatggtcac |
| | aagaccccacgatgttatataacaagatggaattcgagccatttcagtctctggtccctaaggcagccagaggtcagtac |
| | agtgggttcgtgaggacactgttccagcagatgcgggatgtgcttggaactttcgacactgttcagataataaaacttct |
| | tccctttgctgctgctccaccagaacaaagtaggatgcaattctcctccttgactgtaaatgtaaggggatcaggaatga |
| | gaatactagtaaggggcaattctccagtgttcaattacaataaggccactaaaaggcttacagttctcggaaaagatgca |
| | ggtacactaaccgaagatccagatgaaggcacagccggagtagagtctgctgttttgagaggattcctcatcttgggcaa |
| | agaagacaagagatatggcccagcattgagtatcaatgagctgagcaatcttgcaaaaggagagaaggctaatgtgctaa |
| | ttgggcaaggagacgtggtgttggtaatgaaacggaaacgggactctagcatacttactgacagccagacagcgaccaaa |
| | agaattcggatggccatcaattagtgtcgaattgtttaaaacgaccttgtttct |
| SEQ ID NO: 2 | Nucleotide sequence of PB1 viral segment of A/Chicken/Ohio/494832/2007 |
| | ttttcttgaagttccagcgcaaaatgccataagcaccacattcccatatactggagatcctccatacagccatggaacag |
| | gaacaggatacaccatggacacagttaacagaacacatcaatactcagaaaagggaaaatggacaacaaactcagagact |
| | ggagcaccccaacttaacccaattgatggaccactgcccgaggacaatgagccaagtggatatgcacaaacggactgtgt |
| | ccttgaagcaatggctttccttgaagagtctcacccaggaatctttgaaaactcgtgtcttgaaacgatggaagttgttc |
| | aacaaacaagagtggacaaattgacccaaggtcgtcagacctatgattggacattaaacaggaatcagccggctgcaact |
| | gcattagctaatactatagaggtcttcagatcaaacggtctgacagccaatgaatcaggaaggttaatagattttctcaa |
| | ggatgtgatggaatcaatggataaagaggaaatggaaataacaacgcatttccaaaggaaaagaagagtgagagacaaca |
| | tgacaaagaaaatggtcacacaaagaacaataggaaagaagaagcagaggctaaacaaaaggaactacttaataagagca |
| | ttgacactgaacacaatgacaaaagacgccgaaagaggcaaactaaagagaagagcaattgcaacacccggaatgcaaat |
| | cagaggatttgtatactttgttgaaacattagcaaggagcatttgtgagaagcttgaacaatccggactcccagttggag |
| | gcaatgaaaagaaggctaaactggcaaatgttgtgagaaaaatgatgactaattcacaagacacagaactctctttcaca |
| | atcaccggggacaacaccaaatggaatgaaaatcaaaaccctaggatgtttctggcaatgataacatatataacaagaag |
| | ccaacctgaatggtttaggaatgtcttgagcattgcacctataatgttctcaaataaaatggcaagactagggaagggat |
| | acatgttcgaaagtaagagcatgaagcttcgaacacaaataccggcagagatgctagcaagtattgatttgaaatatttc |
| | aatgaatcaacaagaaagaaaatagagaagataagacctcttctaatagatggtacagcctcattgagtcccggaatgat |
| | gatgggcatgttcaacatgttaagtacagttttgggggtttcgattctaaatctagggcaaaagaggtacaccaagacaa |
| | catactggtgggacggacttcaatcctccgatgactttgctctcatagtgaatgctccgaatcatgagggaatacaagca |
| | ggagtagacagattctatagaacctgcaagctggtcggaatcaacatgagcaagaagaagtcctacataaacaggacagg |
| | aacatttgaattcacaagctttttctatcgttatggatttgtagccaactttagcatggagttgcccagctttagcgtgt |
| | ctgggatcaatgaatctgcagacatgagcattggagtaacagtgataaagaacaacatgataaacaatgatcttggacca |
| | gcaacagctcaaatggctcttcagctgttcatcaaggattacagatacacgtatcggtgtcacaggggggacacacaaat |
| | tcagacaaggagatcattcgagctgaagaagttgtgggaacagacccgctcaaaagcaggactgctggtctcagatggag |
| | gaccaaatctatacaatatccggaatctccacattccggaagtctgcttgaaatgggagctaatggatgaagactaccag |
| | ggaaggctttgtaaccccctgaatccgtttgtcagccacaaagagatagagtctgtaaacaatgctgtggtgatgccagc |
| | tcacggcccagccaaaagcatggaatatgatgctgttgctaccacgcactcctggatccctaagaggaatcgctccatcc |
| | tcaatacaagccagaggggaatccttgaagatgaacagatgtatcaaaagtgctgcaatctgttcgagaaattcttccct |
| | agcagttcatacaggagaccggttgggatttcaagcatggtggaggccatggtttctagggcccgaattgatgcgcgaat |
| | tgacttcgaatctggacggattaagaaagaggaatttgctgagatcatgaagatctgttccaccattgaagagctcagac |
| | ggcagaaatagtgaatttagcttgtccttcatgaaaaaatgccttgtttctact |
| SEQ ID NO: 3 | Nucleotide sequence of PA viral segment of A/Chicken/Ohio/494832/2007 |
| | tagcaggtactgatccaaaatggaagattttgtgcgacaatgcttcaatccaatgatcgtcgagcttgcggaaaaggcaa |
| | tgaaagaatatggggaagatccaaaaatcgagacaaacaaattcgctgcaatatgcacacacttggaagtgtgtttcatg |
| | tattcagatttccatttcattgatgaacgaggtgagtcgataatcgtggagtctggcgatccaaatgcactcctaaaaca |
| | ccggtttgagataatcgaagggagagatcgtactatggcctggacagtagtgaatagtatttgcaacaccacaggggttg |
| | aaaaacccaagtttctcccggatttatacgattacaaagagaatcgtttcattgaaattggagtaaccaggagggaagtc |
| | catatatactatttggaaaaggccaataaaataaaatctgagaagacacacatccacatcttttcattcactggagaaga |
| | aatggctactaaagcagactacactcttgatgaagaaagtagagcaaggatcaaaactagactattcaccataagacaag |
| | aaatggccagtagaggcctctgggattcctttcgtcagtccgagagaggcgaagagacaattgaagaaagatttgaaatt |
| | acaggaaccatgcgcaggctcgccgaccaaagtctcccaccgaacttctccagccttgaaaactttagagcctatgtgga |
| | tggattcgaaccaaacggctgcattgagggcaaactttctcaaatgtccaaggaagtaaatgctagaattgaaccattct |
| | tgaagacaacaccacgccccctgaggttaccggaagggcctccttgttcccagcggtcgaaatttctgctgatggatgct |
| | ctgaagcttagcattgaagacccaagtcatgagggcgaggggataccgctgtatgatgcaatcaaatgcatgaaaacctt |
| | tttcggctggaaagagcctaacattgtcaagccacatgaaaagggcataaaccccaattatctcctggcttggaagcaag |
| | tgctagctgagctacaggatattgaaaatgaggagaagattccgaaaacgaagaacatgaagaaaacaagccaattgaag |
| | tgggcacttggtgaaaacatggcaccagagaaagtggactttgaggactgcaaagatgtaagcgacttgagacagtatga |
| | cagcgatgagcctgagcaaagatcactagcaagttggattcagagtgagttcaacaaggcttgtgaattaactgactcaa |
| | gttggatagaactcgatgagataggagaggatgttgccccaatcgaacacattgcaagcatgaggagaaattactttaca |
| | gctgaggtgtctcactgcagggcaacagaatatataatgaaaggagtatacataaacacagccttgctcaacgcttcttg |
| | cgcagcaatggatgatttccagttgatcccaatgataagcaaatgcagaaccaaagaagggcggcggaaaacaaatctgt |
| | atgggttcatcataaagggaaggtctcatttgaggaacgatactgatgtagtgaattttgtgagcatggagttttctctt |
| | actgaccctagactagagccacataaatgggagaagtactgtgtccttgaaataggggacatgctcctacgaactgcaat |
| | aggccaagtgtcgagacccatgtttctatatgtgaggaccaatggaacttccaaaatcaaaatgaaatggggtatggaaa |
| | tgagacgttgtcttcttcaatcacttcagcaaattgaaagcatgattgaggccgagtcttctgtcaaagaaaaagacatg |
| | accaaagagttctttgaaaacaaatcagagacatggcccattggggaatcacccaaaggagtggaagaaggctccatcgg |
| | aaaggtgtgcaggactctgctggcaaaatctgtattcaacagtttgtatgcatctccacaactcgagggattttcagctg |
| | agtctagaaagctgctactcattgttcaggcacttagggacaacctggaacctggtaccttcgatcttggagggctatat |
| | gaagcaattgaggagtgcctgattaatgatccctgggttttgcttaatgcatcttggtcaa |
| SEQ ID NO: 4 | Nucleotide sequence of HA viral segment of A/Chicken/Ohio/494832/2007 |
| | aaaagcaggggttataccatagacaaccgaacaaagacaatgaccatcacttttctcatcctectgttcacagtagtgaa |
| | aggggaccaaatatgtatoggataccatgccaacaattccacagaaaaagttgacacaatcttggaacgaaacgtcaccg |
| | tgactcatgccaaggacattcttgaaaagacgcataatggaaagttgtgcagattaagcgggatccctccattggaactg |
| | ggggattgcagcattgcaggttggctccttggaaatccagagtgtgaccggctcttaagtgtacctgaatggtcctatat |
| | agtggaaaaggaaaacccggtgaatggtctgtgctacccaggcagtttcaatgactatgaggaattgaaacatctcctca |
| | ccagtgtgacacactttgagaaagttaagatcctgcctagagatcaatggacccagcacacaacaactggtggttctcgg |
| | gcctgtgcagtatttgggaatccatcattcttcagaaacatggtctggcttacaaagaaggggtcaaactacccaatcgc |
| | taaaaggtcatacaacaacacaagtggggagcaaatgctgataatctggggggtacatcatcccaatgacgatgcagaac |
| | aaaggacactgtaccagaatgtgggaacatatgtttccgttggaacatcaacactaaataagaggtcaatccctgaaata |
| | gcgacaaggcccaaagtcaatggacaaggagggagaatggaattctcttggactctattagagacatgggatgtcataaa |
| | ttttgagagcactggtaatttaattgcaccagaatatggattcaaaatatcaaagagaggaagctcaggaattatgaaga |
| | cagagaaaacacttgaaaattgtgagaccagatgccagacccccttaggggcaataaatacaacattgccctttcacaac |
| | attcacccattgacaataggtgagtgccccaagtatgtcaagtcagatagactggtcttggcgacaggactaagaaatgt |
| | tcctcagattgaatcaaggggattgtttggagcaatagctgggtttatagaaggcggatggcaagggatggttgatggct |
| | ggtatgggtatcatcacagcaatgatcaaggatcaggatatgcagcagacaaagaatccactcaaaaggcaattgacggg |
| | ataactaacaaggtaaattctgtgattgaaaagatgaacactcagtttgaggctgttgggaaagagttcaacaatctaga |
| | gagaagactagaaaacttaaataagaaaatggaagatggatttctcgatgtatggacatacaatgccgaactectagttc |
| | taatggaaaatgagaggacacttgatttccatgactctaatgtgaggaatctgtacgataaggtcagaatgcaactgagg |
| | gacaatgctaaggaaatagggaacggatgctttgagttttatcataaatgtgatgatgaatgcatgaacagtgtcaggaa |
| | tggaacatatgattatcccaaatatgaagaagagtccaagctgaacaggaacgaaatcaaaggggtgaaattgagcgata |
| | tgggggtctatcaaatacttgctatatacgctacagttgcaggctccttgtcactggcaatcatgatagctgggatttct |
| | ttctggatgtgttctaatgggtctctgcaatgcagaatttgcatttgactgtgagtcaatttgtaattaaaaacaccctt |
| | gtttctact |
| SEQ ID NO: 5 | Nucleotide sequence of NP viral segment of A/Chicken/Ohio/494832/2007 |
| | tagcaaaaggcagggtagataatcactcaccgagtgacatccacatcatggcgtctcaaggcaccaaacgatcttatgag |
| | cagatggaaactggtggagaacgccagaatgccactgaaatcagagcatctgttgggagaatggttggtggaatcgggag |
| | attctatatacagatgtgcactgaactcaaactcagtgactatgaagggagactaatccaaaacagcatcacaatagaga |
| | gaatggttctctcagcatttgatgagagaagaaataaatatctggaagagcatcccagtgctgggaaagaccccaagaag |
| | actggaggcccaatctacaggaggagagatgggaaatggatgagagaattgatcctatatgacaaagaggagatcagaag |
| | gatttggcgtcaagcgaataatggagaagacgcaactgctggtctcacccatttgatgatctggcactccaatctgaatg |
| | atgccacctaccagagaccgagagcacttgtgcgtactgggatggatcccaggatgtgttctctgatgcaaggctcaact |
| | cttccgaggaggtctggagctgctggagcagcagtaaaaggagttggaacaatggtgatggaattgatccgaatgatcaa |
| | gcgagggatcaatgatcggaatttctggagaggcgaaaatgggcggaggacaagaattgcttatgaaagaatgtgcaaca |
| | tcctcaaagggaagtttcaaacagcagcacaacgagcaatgatggaccaggtgagggaaagccggaatcctgggaatgct |
| | gaaattgaagatctcatctttcttgcacggtctgctctcattctgagggggtcagtggctcataagtcttgcctgcctgc |
| | ttgtgtgtatggacttgctgtggccagtggatacgactttgaaagggagggatactccctagtcggaattgatcctttcc |
| | gtctgctccaaaacagtcaagtctttagtcttatcagaccaaacgaaaatccagcacataaaagtcagctggtatggatg |
| | gcatgccactctgcagcttttgaagatctgagagtgtcaagcttcatcagaggaacaagggtagtcccaagaggacaact |
| | gtccaccagaggagtccagattgcttcaaatgagaacatggagacaatggactccagtactcttgaactgaggagcagat |
| | actgggctataaggaccagaagcggaggaaacactaaccagcagagagcatccgcagggcaaatcagcgtacagcccacg |
| | ttctctgtacagagaaacctcccattcgagagagcaaccattatggcggcatttacaggaaacactgaaggcagaacttc |
| | agacatgagaacagaaatcataaggatgatggaaaatgccagacctgaagatgtgtctttccaggggggggagtcttcg |
| | agctctcggacgaaaaggcaacgaacccgatcgtgccttcctttgacatgagtaacgaaggatcttatttetteggagac |
| | aatgcagaggaatatgacaattaaagaaaaatacccttgtttctactg |
| SEQ ID NO: 6 | Nucleotide sequence of NA viral segment of A/Chicken/Ohio/494832/2007 |
| | ttagcaggacgaagatgaatccgaatcagaagataataacaatcggggtagtgaataccactctgtcaacaatagccctt |
| | ctcattggagtggggaacttagttttcaacacagtcatacatgagaaaataggagaccatcaaatagtgacccacccaac |
| | aataacgacccctgcagtaccgaactgcagtgacactataataacatacaataatactgtgataaacaacataacaacaa |
| | caataacaactgaagcagaaaggcctttcaagtccccactaccgctgtgccccttcagaggattcttcccttttcacaag |
| | gacaatgcaatacgactgggtgagaataaagacgtcatagtcacaagggagccttatgttagctgcgataatgacaactg |
| | ctggtcctttgctctcgcacaaggagcattgctagggactaaacatagcaatgggaccattaaagacaggacaccatata |
| | ggtctctaattcgtttcccaataggaacagctccagtactaggaaattacaaagagatatgcattgcttggtcgagcagc |
| | agttgctttgacgggaaagagtggatgcatgtgtgcatgacagggaacgataatgatgcaagtgcccagataatatatgg |
| | agggagaatgacagactccattaaatcatggagaaaggacatactaagaacccaggagtctgaatgtcaatgcattgacg |
| | ggacttgtgttgttgctgtcacagatggccctgctgctaatagtgcagatcacagggtttactggatacgggaaggaaga |
| | ataataaagtatgaaaatgttcccaaaacaaagatacaacatttagaagaatgctcctgctatgtagacattgatgttta |
| | ctgtatatgtagggacaattggaagggctctaacagaccttggatgagaatcaacaacgagactatactggaaacagggt |
| | atgtatgtagtaaattccactcagacacccccaggccagctgatccttcaacaatgtcatgtgactccccaagcaatgtc |
| | aatggaggacccggagtgaaggggtttggtttcaaagctggcaatgatgtatggttaggtagaacagtgtcaactagtgg |
| | tagatcgggctttgaaattatcaaagttacagaagggtggatcaactctcctaaccatgtcaaatcaattacacaaacac |
| | tagtgtccaacaatgactggtcaggctattcaggtagcttcattgtcaaagccaaggactgttttcagccctgtttttat |
| | gttgagcttatacgagggaggcccaacaagaatgatgacgtctcttggacaagtaatagtatagttactttctgtggact |
| | agacaatgaacctggatcgggaaattggccagatggttctaacattgggtttatgcccaagtaatagaaaaaagcacctt |
| | gtttctact |
| SEQ ID NO: 7 | Nucleotide sequence of M viral segment of A/Chicken/Ohio/494832/2007 |
| | gatattgaaagatgagtcttctaaccgaggtcgaaacgtacgttctctctategtcccatcaggccccctcaaagccgag |
| | atcgcgcagagacttgaagatgtgtttgcagggaagaacaccgatcttgaggcactcatggaatggctaaagacaagacc |
| | aatcctgtcacctctgactaaggggattttaggatttgtgttcacgctcaccgtgcccagtgagcgaggactgcagogta |
| | gacgctttgtccaaaatgcccttaatgggaatggggatccaaacaacatggacagagcggtcaaactgtacaggaagctt |
| | aaaagggagataacattccatggggcaaaagaagtggcactcagttattctactggtgcacttgccagttgcatgggcct |
| | catatacaacagaatggggactgtgaccactgaagtggcatttggcctagtgtgcgccacgtgtgagcagattgctgatt |
| | cccagcatcggtctcacagacaaatggtgacaacaacaaacccactaatcaggcacgagaacagaatggtactggccagt |
| | accacggctaaggccatggagcaaatggcagggtcaagtgagcaggcagcagaggctatggaggttgctagtcaggctag |
| | acagatggtgcaggcaatgaggaccattgggactcatcctagctccagtgctggtctgaaagatgatcttcttgaaaatt |
| | tgcaggcctatcagaaacggatgggagtgcagatgcagcgattcaagtgatcctctcgttattgccgcaagtatcattgg |
| | gatcttgcacttgatattgtggattcttgatcgtcttttcttcaaatgcatttategtcgccttaaatacggtttgaaaa |
| | gagggccttctacggaaggagtgcctgagtctatgagggaagaatatcggcaggaacagcagagtgctgtggatgttgac |
| | gatggtcattttgtcaacatagagctggagtaaaaaactaccttgtttctactaa |
| SEQ ID NO: 8 | Nucleotide sequence of NS viral segment of A/Chicken/Ohio/494832/2007 |
| | gggtgacaaaaacataatggattccaacactgtgtcaagctttcaggtagactgctttctttggcatgtccgcaaacgat |
| | ttgcagaccaagaattgggtgatgccccattccttgaccggcttcgccgagaccagaagtctctaagaggaagaggcagc |
| | actcttggtctggacatcgaaacggccactcgtgctggaaagcagatagtggagagggttctggaggaagaatccgacga |
| | ggcactcaaaatgactattgcctctgtgcctgcttcacgctacctaactgacatgactcttgaagagatgtcaagagact |
| | ggttcatgctcatgcccaagcaaaaagtggcaggctccctctgtatcagaatggaccaggcgattatggataagaacatc |
| | atactgaaggcaaacttcagtgtgatcttcgatcggctggagacactaatactactcagagctttcactgaagagggagc |
| | aattgtcggcgaaatttcaccattgccttctcttccaggacatactgatgaggatgtcaaaaatgcaattggggtcctca |
| | tcggaggacttgaatggaatgataacacagttcgagtctctgaaactttacagagattcgcttggagaagcagtaatgag |
| | gatgggagacctccactccctccaaagcagaaacggaaaatggcgagaacaattgagtcagaagtttgaagaaataaggt |
| | ggctgattgaagaagtgcgacatagactaaagatcacagaaaatagttttgaacaaataacatttatgcaagccttacaa |
| | ctactgcttgaagtggagcaagagataagaactttctcgtttcagcttatttaatgataaaaaacaccctg |
| SEQ ID NO: 9 | Amino acid sequence of PB2 protein of A/Chicken/Ohio/494832/2007 |
| | LRDLMSQSRTREILTKTTVDHMAIIKKYTSGRQEKNPALRMKWMMAMKYPITADKRIMEMIPERNEQGQTLWSKTNDAGS |
| | DRVMVSPLAVTWWNRNGPTTSTVHYPKVYKTYFEKVKRLKHGTFGPVHFRNQVKIRRRVDINPGHADLSAKEAQDVIMEV |
| | VFPNEVGARILTSESQLTITREKKEELQDCKIAPLMVAYMLERELVRKTRFLPVAGGTSSVYIEVLHLTQGTCWEQMYTP |
| | GGEVRNDDVDQSLIIAARNIVRRATVSADPLASLLEMCHSTQIGGIRMVDILRQNPTEEQAVDICKAAMGLRISSSFSFG |
| | GFTFKRTSGSSVKKEEEVLTGNLQTLKIRVHEGYEEFTMVGRRATAILRKATRRLIQLIVSGRDEQSIAEAIIVAMVFSQ |
| | EDCMIKAVRGDLNFVNRANQRLNPMHQLLRHFQKDAKVLFQNWGIEPIDNVMGMIGILPDMTPSTEMSLRGIRVSKMGVD |
| | EYSSTERVVVSIDRFLRVRDQRGNVLLSPEEVSETQGTEKLTITYSSSMMWEINGPESVLVNTYQWIIRNWETVKIQWSQ |
| | DPTMLYNKMEFEPFQSLVPKAARGQYSGFVRTLFQQMRDVLGTFDTVQIIKLLPFAAAPPEQSRMQFSSLTVNVRGSGMR |
| | ILVRGNSPVFNYNKATKRLTVLGKDAGTLTEDPDEGTAGVESAVLRGFLILGKEDKRYGPALSINELSNLAKGEKANVLI |
| | GQGDVVLVMKRKRDSSILTDSQTATKRIRMAIN |
| SEQ ID NO: | Amino acid sequence of PB1 protein of A/Chicken/Ohio/494832/2007 |
| 10 | FLEVPAQNAISTTFPYTGDPPYSHGTGTGYTMDTVNRTHQYSEKGKWTTNSETGAPQLNPIDGPLPEDNEPSGYAQTDCV |
| | LEAMAFLEESHPGIFENSCLETMEVVQQTRVDKLTQGRQTYDWTLNRNQPAATALANTIEVFRSNGLTANESGRLIDFLK |
| | DVMESMDKEEMEITTHFQRKRRVRDNMTKKMVTQRTIGKKKQRLNKRNYLIRALTLNTMTKDAERGKLKRRAIATPGMQI |
| | RGFVYFVETLARSICEKLEQSGLPVGGNEKKAKLANVVRKMMTNSQDTELSFTITGDNTKWNENQNPRMFLAMITYITRS |
| | QPEWFRNVLSIAPIMFSNKMARLGKGYMFESKSMKLRTQIPAEMLASIDLKYFNESTRKKIEKIRPLLIDGTASLSPGMM |
| | MGMFNMLSTVLGVSILNLGQKRYTKTTYWWDGLQSSDDFALIVNAPNHEGIQAGVDRFYRTCKLVGINMSKKKSYINRTG |
| | TFEFTSFFYRYGFVANFSMELPSFSVSGINESADMSIGVTVIKNNMINNDLGPATAQMALQLFIKDYRYTYRCHRGDTQI |
| | QTRRSFELKKLWEQTRSKAGLLVSDGGPNLYNIRNLHIPEVCLKWELMDEDYQGRLCNPLNPFVSHKEIESVNNAVVMPA |
| | HGPAKSMEYDAVATTHSWIPKRNRSILNTSQRGILEDEQMYQKCCNLFEKFFPSSSYRRPVGISSMVEAMVSRARIDARI |
| | DFESGRIKKEEFAEIMKICSTIEELRRQK |
| SEQ ID NO: | Amino acid sequence of PA protein of A/Chicken/Ohio/494832/2007 |
| 11 | MEDFVRQCFNPMIVELAEKAMKEYGEDPKIETNKFAAICTHLEVCFMYSDFHFIDERGESIIVESGDPNALLKHRFEIIE |
| | GRDRTMAWTVVNSICNTTGVEKPKFLPDLYDYKENRFIEIGVTRREVHIYYLEKANKIKSEKTHIHIFSFTGEEMATKAD |
| | YTLDEESRARIKTRLFTIRQEMASRGLWDSFRQSERGEETIEERFEITGTMRRLADQSLPPNFSSLENFRAYVDGFEPNG |
| | CIEGKLSQMSKEVNARIEPFLKTTPRPLRLPEGPPCSQRSKFLLMDALKLSIEDPSHEGEGIPLYDAIKCMKTFFGWKEP |
| | NIVKPHEKGINPNYLLAWKQVLAELQDIENEEKIPKTKNMKKTSQLKWALGENMAPEKVDFEDCKDVSDLRQYDSDEPEQ |
| | RSLASWIQSEFNKACELTDSSWIELDEIGEDVAPIEHIASMRRNYFTAEVSHCRATEYIMKGVYINTALLNASCAAMDDF |
| | QLIPMISKCRTKEGRRKTNLYGFIIKGRSHLRNDTDVVNFVSMEFSLTDPRLEPHKWEKYCVLEIGDMLLRTAIGQVSRP |
| | MFLYVRTNGTSKIKMKWGMEMRRCLLQSLQQIESMIEAESSVKEKDMTKEFFENKSETWPIGESPKGVEEGSIGKVCRTL |
| | LAKSVFNSLYASPQLEGFSAESRKLLLIVQALRDNLEPGTFDLGGLYEAIEECLINDPWVLLNASWS |
| SEQ ID NO: | Amino acid sequence of HA protein of A/Chicken/Ohio/494832/2007 |
| 12 | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNKVNSVIEKMN |
| | TQFEAVGKEFNNLERRLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Amino acid sequence of NP protein of A/Chicken/Ohio/494832/2007 |
| 13 | MASQGTKRSYEQMETGGERQNATEIRASVGRMVGGIGRFYIQMCTELKLSDYEGRLIQNSITIERMVLSAFDERRNKYLE |
| | EHPSAGKDPKKTGGPIYRRRDGKWMRELILYDKEEIRRIWRQANNGEDATAGLTHLMIWHSNLNDATYQRPRALVRTGMD |
| | PRMCSLMQGSTLPRRSGAAGAAVKGVGTMVMELIRMIKRGINDRNFWRGENGRRTRIAYERMCNILKGKFQTAAQRAMMD |
| | QVRESRNPGNAEIEDLIFLARSALILRGSVAHKSCLPACVYGLAVASGYDFEREGYSLVGIDPFRLLQNSQVFSLIRPNE |
| | NPAHKSQLVWMACHSAAFEDLRVSSFIRGTRVVPRGQLSTRGVQIASNENMETMDSSTLELRSRYWAIRTRSGGNTNQQR |
| | ASAGQISVQPTFSVQRNLPFERATIMAAFTGNTEGRTSDMRTEIIRMMENARPEDVSFQGRGVFELSDEKATNPIVPSFD |
| | MSNEGSYFFGDNAEEYDN |
| SEQ ID NO: | Amino acid sequence of NA protein of A/Chicken/Ohio/494832/2007 |
| 14 | MNPNQKIITIGVVNTTLSTIALLIGVGNLVFNTVIHEKIGDHQIVTHPTITTPAVPNCSDTIITYNNTVINNITTTITTE |
| | AERPFKSPLPLCPFRGFFPFHKDNAIRLGENKDVIVTREPYVSCDNDNCWSFALAQGALLGTKHSNGTIKDRTPYRSLIR |
| | FPIGTAPVLGNYKEICIAWSSSSCFDGKEWMHVCMTGNDNDASAQIIYGGRMTDSIKSWRKDILRTQESECQCIDGTCVV |
| | AVTDGPAANSADHRVYWIREGRIIKYENVPKTKIQHLEECSCYVDIDVYCICRDNWKGSNRPWMRINNETILETGYVCSK |
| | FHSDTPRPADPSTMSCDSPSNVNGGPGVKGFGFKAGNDVWLGRTVSTSGRSGFEIIKVTEGWINSPNHVKSITQTLVSNN |
| | DWSGYSGSFIVKAKDCFQPCFYVELIRGRPNKNDDVSWTSNSIVTFCGLDNEPGSGNWPDGSNIGFMPK |
| SEQ ID NO: | Amino acid sequence of M1 protein of A/Chicken/Ohio/494832/2007 |
| 15 | MSLLTEVETYVLSIVPSGPLKAEIAQRLEDVFAGKNTDLEALMEWLKTRPILSPLTKGILGFVFTLTVPSERGLQRRRFV |
| | QNALNGNGDPNNMDRAVKLYRKLKREITFHGAKEVALSYSTGALASCMGLIYNRMGTVTTEVAFGLVCATCEQIADSQHR |
| | SHRQMVTTTNPLIRHENRMVLASTTAKAMEQMAGSSEQAAEAMEVASQARQMVQAMRTIGTHPSSSAGLKDDLLENLQAY |
| | QKRMGVQMQRFK |
| SEQ ID NO: | Amino acid sequence of M2 protein of A/Chicken/Ohio/494832/2007 |
| 16 | MSLLTEVETPIRNGWECRCSDSSDPLVIAASIIGILHLILWILDRLFFKCIYRRLKYGLKRGPSTEGVPESMREEYRQEQ |
| | QSAVDVDDGHFVNIELE |
| SEQ ID NO: | Nucleotide sequence of PB2 viral segment of A/Swine/Missouri/2124514/2006 |
| 17 | tatattcaatatggagagaataaaagaactaagagacctaatgtegcagtcccgcactegegaaatactcaccaagacca |
| | ctgtggaccatatggccataatcaaaaagtacacatcgggaaggcaagagaagaaccctgcactcagaatgaagtggatg |
| | atggcaatgaagtacccaatcacagcagacaagagaataatggacatgattccagagagaaatgaacaaggacaaaccct |
| | ctggagcaaaacaaacgatgctggatcggaccgcgtgatggtatcacccctggctgtaacatggtggaataggaatggcc |
| | caacagcaagcacagttcactaccctaaggtatataaaacttatttogaaaaagtcgaaaggttaaaacatggtaccttt |
| | ggccctgtccacttcaggaatcaagttaaaataagaaggagagttgacacaaaccctggtcacgcagatctcagagccaa |
| | ggaggcacaggatgtgatcatggaagttgttttcccaaatgaagtgggggcaagaatactgacatcagagtcacagctga |
| | caataacaaaagagaagaaagaagagctccaggattgtaaaattgctcccttaatggtggcatacatgctagaaaaagag |
| | ttggtccgtaaaacgaggtttctcccggtggctggtggaacaggcagtgtttatattgaagtgctgcatttaactcaggg |
| | gacatgctgggagcaaatgtacactccaggaggagaagtgagaaatgatgatgttgaccaaagtttgattattgctgcta |
| | gaaacatagtaagaagagcagcagtgtcagcagacccattagcatctcttttggaaatgtgccacagcacacagattgga |
| | ggaataaggatggtggacatccttagacagaatccaacggaggaacaagccgtagacatatgcaaggcagcaatggggct |
| | gaggattagctcctctttcagctttggtgggttcactttcaaaagaacaagtgggtcatcagttaagagagaagaagaag |
| | tgctcacgggcaaccttcaaacactgaaaataagagtacatgaaggatatgaagaattcacaatggtcgggagaagagca |
| | acagctattctcagaaaagcaaccaggagattgatccagttgatagtaagtgggagagacgagcagtcaattgctgaggc |
| | aataattgtggccatggtattttcacaagaggactgcatgatcaaggcagttaggggcgatctgaactttgtcaataggg |
| | caaaccagcgactgaatcccatgcatcaactcttgaggcatttccaaaaagatgcaaaagtgcttttccagaattgggga |
| | attgaacccatcgacaatgtgatgggaatgatcgggatattgcccgatatgaccccaagcacggagatgtcgctgagagg |
| | gataagagtcagcaaaatgggagtagatgaatactccagcacggagagagtggtagtgagcattgacagatttttgaggg |
| | ttcgggatcaacgagggaacgtactattgtctcctgaagaggtcagtgagacacaagggacggagaaattggcaataact |
| | tattcgtcatcgatgatgtgggagatcagtggccctgagtcagtgctggtcaacacttatcaatggatcataaggaattg |
| | ggaaagtttgaaaattcaatggtcacaagatcccacgatgttatacaacaaaatggaatttgaaccatttcagtctcttg |
| | tccctaaagcaaccagaagtcgttacagtgggttcgtgaggacactgttccagcaaatgcgggatgtgattggaacattt |
| | gacactgtccaaataataaaacttctcccctttgctgctgctccaccagaacagagtaggatgcagttttcctcactgac |
| | tgtgaatgtgagaggatcagggttgaggatactggtaagaggcaattctccagtattcaattacaataaagcaaccaaaa |
| | ggcttacagttcttggaaaggatgcaggtgaattgaccgaagacccagatgaaggcacagctggagtggagtctgctgtc |
| | ctgaggggatttctcattttgggcaaagaagacaagagatatggtccagcattaagcatcaatgaactgagcaatcttgc |
| | aaaaggagagaaagctaatgtgctaattgggcaaggagacgtagtgttggtaatgaaacggaaacgggactctagcatac |
| | ttactgacagccagacagcgaccaaaagaattcggatggccatcaattagtgtcgaatt |
| SEQ ID NO: | Nucleotide sequence of PB1 viral segment of A/Swine/Missouri/2124514/2006 |
| 18 | ttgaatggatgtcaatccgaccctacttttcctaaaagttccagcgcaaaatgccataagcaccacattcccttatactg |
| | gagatcctccatacagccatggaacaggaacaggatacaccatggacacagtcaacagaacacatcaatattcagaaaaa |
| | gggaagtggacgacaaacactgagactggggcaccccaactcaatccaattgatggaccactacctgaggataatgaacc |
| | aagtgggtatgcacagacagactgtgttctagaggccatggctttccttgaagaatcccacccagggatatttgagaatt |
| | catgccttgaaacaatggaagttgttcaacaaacaagggtggataaactaactcaaggtcgccagacttatgattggaca |
| | ttaaacagaaatcaaccagcagcaactgcattggccaacaccatagaagtttttaggtcgaatagtctaacagccaatga |
| | gtcaggaagactaataaatttcctaaaggatgtaatggaatcaatggatagagaagaagtggagataacaacacactttc |
| | gaagaaaaaggagagtgagagacaacatgaccaagaagatggtcacacaaagaacaataggaaagaaaaaacaaagattg |
| | agtaagagaagttatctaataagagcacttacattgaatacgatgaccaaagatgcagagagaggcaaattaaaaagaag |
| | ggctatcgcaacacccgggatgcaaattagagggttcgtgtactttgttgagactttagctaggagcatttgcgaaaagc |
| | ttgaacagtctggactcccagtagggggaaatgaaaagaaggccaaattggcaaatgttgtgagaaaaatgatgactaat |
| | tcacaagacacagagctttctttcacaatcactggggacaatactaagtggaatgaaaatcaaaatcctcgaatgttcct |
| | ggcgatgattacatatatcacccgaaatcaacccgagtggttcagaaacatcctgagcatggcacccataatgttttcaa |
| | acaaaatggcaagactaggaaaagggtacatgttcgagagtaaaaggatgaagctccgaacacaaataccagcagaaatg |
| | ctagcaagcattgatctgaagtatttcaatgaatcaacaaggaagaaaattgagaaaataaggcctcttctaatagatgg |
| | cacagcatcattgagccctggaatgatgatgggcatgttcaacatgctaagtacggttttgggagtctcgatactgaatc |
| | ttggacaaaagaaatacaccaggacaacatactggtgggatgggctccaatcctccgacgattttgccctcattgtgaat |
| | gcaccaaattatgagggaatacaagcaggagtgaatagattctacaggacctgcaagttagtaggaataaacatgagcaa |
| | aaagaagtcctatataaataaaacagggacatttgaattcacaagctttttttatcgctatgggtttgtggctaatttta |
| | gcatggagctgcccagttttggagtgtctggaataaatgaatcagctgatatgagtattggagtaacagtgataaagaac |
| | aatatgataaacaatgatcttggacctgcaacagcccagatggcccttcaattgttcatcaaagactacagatacacata |
| | taggtgccacagaggagacacacaaattcagacgagaagatcattcgagctaaagaagctatgggatcaaacccgatcaa |
| | atgcaggactattagtatctgatggaggaccaaacttatacaatatcaggaatcttcacattcctgaagtctgcttaaaa |
| | tgggagctaatggatgaggattatcggggaaggctttgtaatcccctgaatccttttgtcagccataaagagattgattc |
| | tgtaaacaatgctgtggtgatgccagcccatggcccagccaaaagcatggaatatgatgccgttgcaactacacactect |
| | ggatccccaagaggaatcgctctattctcaacacaagccaaaggggaattcttgaggatgaacagatgtaccagaagtgc |
| | tgcaacctgttcgagaaatttttccctagtagttcatacaggagaccggttggaatttctagcatggtggaggccatggt |
| | gtctagggcccggattgatgccagaatcgacttcgagtctggacggattaagaaagaagagttctctgagatcataagga |
| | tctgttccaccattgaagaactcagacggcaaacatgatgaatttggcttgtccttcatgaaaaaatgccttgtttctac |
| | taatacgagacgatataaa |
| SEQ ID NO: | Nucleotide sequence of PA viral segment of A/Swine/Missouri/2124514/2006 |
| 19 | ccaaaatggaagactttgtgcgacaatgcttcaatccaatgatcgtcgagcttgcggaaaaggcaatgaaggaatatggg |
| | gaagatccaaaaatcgaaactaacaaattcgcagcaatatgcactcacttggaagtatgtttcatgtattcggatttcca |
| | cttcattgatgagcggggcgaatcaataattgtggaatctggtgatccaaatgcattactgaagcaccgatttgaaataa |
| | ttgaaggaagagaccgaacaatggcctggacagtggtgaatagcatctgcaacaccacaggggtcgagaagcctaaattt |
| | cttccggatctgtatgattacaaggagaaccgattcattgaaattggagtaacacggagagaggttcatatatactacct |
| | agagaaagccaacaagataaaatctgagaagacacacattcacatcttttcatttactggagaagaaatggccaccaaag |
| | cagactacactcttgatgaagaaagcagggcaagaatcaaaaccaggctattcactataagacaagaaatggccagcagg |
| | ggtctatgggattcctttcgtcagtctgaaagaggcgaagagacaattgaggaaagatttgaaatcacaggaaccatgcg |
| | taggcttgccgaccaaagtctcccaccgaacttctccagccttgaaaactttagagcctatgtggatggattegaaccga |
| | acggctgcattgagggcaagctttctcaaatgtcaaaagaagtgaatgccaggattgagccattcctgaagacaacacca |
| | cgccctctcaaattacctgatgggcccccttgctctcagcggtcaaaattcttgctgatggatgccttgaaactaagcat |
| | cgaagacccgagtcacgagggagagggtataccactatacgatgcaatcaaatgcatgaagacatttttcggctggaaag |
| | agcccaacataatcaaaccacatgagaaaggcataaatcccaattactttctggcttggaagcaagtgctagcagagctc |
| | caggaccttgaaaatgaagagaagatcccaaagacaaagaacatgaagaaaacaagccaattgaagtgggcgcttggtga |
| | gaatatggcaccagaaaaagtggactttgaggattgcaaggacattggcgatctaaagcagtatgatagtgatgagccag |
| | agcctagatcgctggcaagctggatccagagtgaattcaataaggcatgtgaattgaccgactcgagctggatagaactt |
| | gatgaaataggagaagatgttgctccgattgaacacattgcaagtatgaggaggaactattttacagcagaagtgtccca |
| | ctgcagggccactgaatacataatgaaaggagtctacataaatacagctctgctcaatgcatcttgtgcagccatggatg |
| | acttccagctgattccaatgataagcaaatgtagaacaaaggaaggaagacggaaaacaaacctgtatgggttcatcata |
| | aaaggaagatctcatttgaggaatgatactgatgtggtaaactttgtgagcatggaattttctctcactgacccgaggct |
| | agaaccacacaaatgggagaagtattgtgttcttgaaataggagatatgctcctgaggactgcaataggccaagtgtcaa |
| | ggcccatgttcctgtacattagaaccaatgggacctccaagatcaagatgaaatggggtatggaaatgaggcgctgcctc |
| | cttcaatctcttcaacagattgagagcatgattgaggctgagtcttctgtcaaagaaaaggacatgactaaggaattctt |
| | tgaaaacaagtcggaaacgtggccaattggagaatcccccagaggagtagaggaaggctctattgggaaagtatgcagaa |
| | ccttactggcaaagtctgtattcaacagtctgtacgcatctccacaacttgaggggttttcagctgaatcgagaaaattg |
| | cttctcattgttcaggcacttagggacaacctggaacctgggaccttcgatcttggggggctatacgaagcaattgagga |
| | gtgcctgattaatgatccctgggttttgcttaatgcatcttggttcaactccttcctcacacatgcactgaaatagttgt |
| | ggcaatgctactatttgctatccatactgtccaaaaaagtaccttgtttctactaatagaagagcgatga |
| SEQ ID NO: | Nucleotide sequence of HA viral segment of A/Swine/Missouri/2124514/2006 |
| 20 | ttattcgtcttcagggagcaaaagcaggggttataccatagacaaccgaacaaagacaatgaccatcacttttctcatcc |
| | tcctgttcacagtagtgaaaggggaccaaatatgcatcggataccatgccaacaattccacagaaaaagttgacacaatc |
| | ttggaacgaaacgtcaccgtgactcatgccaagaacattcttgaaaagacgcataatggaaagttgtgcagattgagcgg |
| | gatccctccattggaactgggggattgcagcattgcaggttggctccttggaaatccggaatgtgaccggctcttaagtg |
| | tacctgaatggtcctatatagtggaaaaggaaaacccggtgaatggtctgtgctatccaggcagtttcaatgattatgag |
| | gaattgaaacatcttctcaccagtgtgacacactttgagaaagttaagattctgcccagagatcaatggacccagcacac |
| | aacaactggtggttctcgggcctgtgcagtatctggaaacccgtcattctttaggaacatggtttggcttacaaagaaag |
| | ggtcaaactacccaattgctaaaaggtcatacaacaacacaagtggggagcaaatgctggtaatatgggggatacatcac |
| | cccaatgacgatgcggaacagaggacactgtaccagaatgtgggaacatatgtttccgttggaacatcaacactaaataa |
| | gaggtcaatccctgaaatagcaacaaggcccaaagtcaatggactgggaggaagaatggaattctcttggactctattgg |
| | agacatgggatgtcataaattttgagagcactggtaatttaattgcaccagaatacggattcaaaatatcaaagagagga |
| | agctcaggaattatgaagacagagaaaatacttgaaaattgtgaaaccaaatgtcagacccccttgggggcaataaatac |
| | aacattgccctttcacaacattcacccattgacaataggtgagtgccccaagtatgtaaagtcagatagactgattttgg |
| | cgacaggactaagaaatgtcccccagattgaatcaaggggattgtttggagcaatagctgggtttatagaaggcggatgg |
| | caagggatggttgatggctggtatgggtaccatcacagcaatgatcaaggatcaggatatgcagcagacaaagaatccac |
| | tcaaaaggcaattgatgggataactaacaaagtaaattctgtgattgaaaagatgaacactcagtttgaggctgttggga |
| | aagagttcaacaacctagagagaagactggaaaacttaaataaaaagatggaagatggatttattgatgtatggacatat |
| | aatgccgaactcctagttctaatggaaaatgagaggacacttgatttccatgattctaatgtgaagaatctgtacgataa |
| | ggtcagaatgcaattgagagacaatgctaaggaaatagggaacggatgctttgagttttatcataaatgtgatgatgaat |
| | gcatgaatagtgtcaggaatgggacatatgattatatcaaatatgaggaagagtccaagctgaacaggaacgaaatcaaa |
| | ggagtgaaattgagcaatatgggggtttatcaaatacttgctatatacgctacagttgcaggctctttgtcactggcaat |
| | catgatagctgggatttctttctggatgtgttctaatgggtctctgcaatgcagaatttgcatatgactgtaagtcaatt |
| | tgtaattaaaaacacccttgtttctactaatacgagacgatataa |
| SEQ ID NO: | Nucleotide sequence of NP viral segment of A/Swine/Missouri/2124514/2006 |
| 21 | acccatcaatgagtgacatcgaagccatggcgtctcaaggcaccaaacgatcatatgaacaaatggagactggtggggaa |
| | cgccaggatgccacagaaatcagagcatctgtcggaagaatgattggtggaatcgggaaattctacatccaaatgtgcac |
| | tgaactcaaactcagtgactatgagggacgactaatccaaaatagcatgacaatagagagaatggtgctctctgcttttg |
| | atgagagaagaaataaatacctagaagagcatcccagtgcagggaaggatcctaagaaaactggaggacccatatataga |
| | agagtagacggaaagtggatgagagaactcattctttatgacaaagaagaaataaggagagtttggcgccaagcaaacaa |
| | tggtgaagatgcaacagctggtcttgctcatatcatgatttggcactccaatctgaatgatgccacgtaccagagaacaa |
| | gagcgcttgttcgcaccggaatggatcccagaatgtgctctctaatgcaaggttcaacacttcccagaaggtctggggcc |
| | gcaggtgctgcagtgaaaggagttggaacaatagcaatggaattaatcagaatgatcaaacgtgggatcaatgaccgaaa |
| | cttctggagaggtgaaaatggacgaaagacaaggattgcatatgaaagaatgtgcaatattctcaagggaaaatttcaga |
| | cagctgcccagagggcaatgatggatcaagtgagagaaagtcggaaccccggaaacgctgagattgaagatctcattttc |
| | ctggcacggtcagcacttattctaaggggatcagttgcacataagtcttgcctgcctgcttgtgtgtatgggcttgcagt |
| | ggcaagtgggcatgactttgaaagggaagggtattcactggtcgggatagacccatttaaattactccaaaacagtcaag |
| | tgttcagcttgataagaccaaatgaaaacccagctcacaagagtcaattagtgtggatggcatgccactctgctgcattt |
| | gaggatctgagggtatcaagtttcatcagagggaagaaagtgattccaagaggaaggctctccacaagaggggttcagat |
| | tgcttcaaatgagaatgtggaagccatggattccaataccttagagctgagaagcagatactgggccataaggaccagaa |
| | gtggaggaaataccaatcaacagaaggcatccgcgggccagatcagtgtgcaacccacattctcagtgcaacggaatctc |
| | ccttttgaaagagcaaccgttatggcagctttcagcgggaacaatgaaggacggacatccgatatgcgaacagaagttat |
| | aaggatgatggaaaatgcaaagccagaagatttgtccttccaggggggggagtcttcgagctctoggacgaaaaagcaa |
| | cgagcccgatcgtgccttcctttgacatgagtaatgaaggatcttatttcttcggagacaatgcagaggagtatgacagt |
| | tgaggaaaaata |
| SEQ ID NO: | Nucleotide sequence of NA viral segment of A/Swine/Missouri/2124514/2006 |
| 22 | cgagatgaatccgaatcagaagataataacaatcggggtagtgaataccactctgtcaacaatagcccttctcattggag |
| | tgggaaacttagttttcaacacagtcatacatgagaaaataggagaccatcaaatagtgacctatecaataataacgacc |
| | cctgcagtaccgaactgcagtgacactataataacatacaataacactgtgataaacaacataacaacaacaataataac |
| | tgaagaagaaaggcctttcaagtctccactaccgctgtgccccttcagaggattettcccttttcacaaggacaatgcaa |
| | tacgactgggtgaaaacaaagacgtcatagtcacaagagagccttatgttagctgcgataatgacaactgctggtccttt |
| | gctctcgcacaaggagcattgctagggaccaaacatagcaatgggaccattaaagacaggacaccatataggtctctaat |
| | tcgtttcccaataggaacagctccagtactaggaaattataaagagatatgcattgcttggtcgagcagcagttgctttg |
| | acgggaaagagtggatgcatgtgtgcatgacagggaacgataatgatgcaagtgcccagataatatatggagggagaatg |
| | acagactccattaaatcatggagaaaggacatactaagaactcaggagtctgaatgccaatgcattgacgggacttgtgt |
| | tgttgctgtcacagatggccctgctgctaatagtgcagattacagggtttactggatacgggagggaaaaataataaagt |
| | atgaaaatgttcccaaaacaaagatacaacacttagaagaatgttcctgctatgtggacattgatgtttactgtatatgt |
| | agggacaattggaagggctctaacagaccttggatgagaatcaacaacgagactatactggaaacagggtatgtatgtag |
| | taaattccactcagacacccccaggccagctgacccttcaacaatgtcatgtgactccccaagcaatgtcaatggaggac |
| | ccggagtgaaggggtttggtttcaaagctggcgatgatgtatggttaggtagaacagtgtcgactagtggtagatcgggc |
| | tttgaaattatcaaagttacagaagggtggatcaactctcctaaccatgtcaaatcaattacacaaacactagtgtccaa |
| | caatgactggtcaggctattccggtagcttcattgtcaaagccaaggactgttttcagccctgtttttatgttgagctta |
| | tacgagggaggcccaacaagaatgatgacgtctcttggacaagtaatagtatagttactttctgtggactagacaatgaa |
| | cctggatcgggaaattggccagatggttctaacattgggtttatgcccaagtaatagaaaaaagcaccttgtttctacta |
| SEQ ID NO: | Nucleotide sequence of M viral segment of A/Swine/Missouri/2124514/2006 |
| 23 | ttaaagatgagtcttctaaccgaggtcgaaacgtatgttctctctatcgtcccgtcaggccccctcaaagccgagatagc |
| | acagagactcgaagacgtttttgcagggaaaaacaccgatcttgaggctctcatggaatggctaaagacaagaccaatcc |
| | tgtcacctctgactaaggggattttagggtttgtgttcacgctcaccgtgcccagtgagcgaggactgcagcgtagacgt |
| | tttgttcagaatgccctcaatgggaatggtgacccgaacaacatggacaaggcggtcaaactttacaggaaactaaaaag |
| | ggaaataacattccatggggccaaagaagtagcgctcagttactctgctggtgcacttgccagttgcatgggcctcatat |
| | acaacagaatgggaactgtcaccactgaggttgcctttggtctggtatgcgcaacctgtgaacagattgctgattctcag |
| | catcgatcccatagacaaatggtgacaacaaccaatccactaatcaggcacgagaacagaatggtgatagccagcacaac |
| | agctaaagcaatggaacaaatggctggatcaagcgaacaagcagcagaggctatggaggttgccagccaggctagacaaa |
| | tggtacaggcaatgagaacaattgggactcaccctagttccagcactggtctaaaagatgatcttcttgaaaatttacag |
| | gcctatcagaagcggatgggagtgcaaatgcaacgattcaaatgatcctctcattgatgctgcaagcatcattgggattt |
| | tgcacctgatattgtggattcttgatcgtctttttttcaaatgcatttaccgtcgctttaaatacggtctgcaaagaggg |
| | ccttctacggaaggagtgccggagtccatgagggaagaatatcgacagaaacagcagagtgctgtggatgttgacgatgg |
| | tcattttgtcaacatagtgctagagtaaa |
| SEQ ID NO: | Nucleotide sequence of NS viral segment of A/Swine/Missouri/2124514/2006 |
| 24 | caaaaagacataatggactccacaactgtgtcaagctttcaggtagactgtttcctttggcacatccgcaaacggtttgc |
| | agacaatggattgggtgatgccccattccttgateggctccgccgagatcaaaagtctctaaaaggaagaggcaacaccc |
| | ttggcctcgacattgaaacagccactcttgttgggaaacaaattgtggagtggattttgaaagaagaatccagcgataca |
| | cttaagatgactattgcatctgtacctacttcgcgctatttagctgacatgaccctcgaggaaatgtcacgagactggtt |
| | aatgctcatgcctaggcaaaagataataggccctctttgtgtgcgaatggaccaggcgatcatggaaaagaacatcatac |
| | tgaaagcgaacttcagtgtgatotttaaccgattagagactttgatactactaagggctttcacagaggagggaacaatc |
| | gttggagaaatttcacaattaccttctcttccaggacatactaatgaggatgtcaaaaatgcaattggggtcctcatcgg |
| | aggacttgaatggaatggtaacacggttcgaggctctgaaaatctacagagattcgcttggagaaaccgtaatgaggatg |
| | ggagaccttcactacctccagagaagaaatgaaaagtggcgagagcaattgggacaaaaatttgaggaaataaggtggtt |
| | aattgaagaagtgcggcacagattgaaaacgacagagaatagttttgaacaaataacattcatgcaagccttacaactac |
| | tgcttgaagtagaacaagagataaggactttctcgtttcagcttatttaat |
| SEQ ID NO: | Amino acid sequence of PB2 protein of A/Swine/Missouri/2124514/2006 |
| 25 | MERIKELRDLMSQSRTREILTKTTVDHMAIIKKYTSGRQEKNPALRMKWMMAMKYPITADKRIMDMIPERNEQGQTLWSK |
| | TNDAGSDRVMVSPLAVTWWNRNGPTASTVHYPKVYKTYFEKVERLKHGTFGPVHFRNQVKIRRRVDTNPGHADLRAKEAQ |
| | DVIMEVVFPNEVGARILTSESQLTITKEKKEELQDCKIAPLMVAYMLEKELVRKTRFLPVAGGTGSVYIEVLHLTQGTCW |
| | EQMYTPGGEVRNDDVDQSLIIAARNIVRRAAVSADPLASLLEMCHSTQIGGIRMVDILRQNPTEEQAVDICKAAMGLRIS |
| | SSFSFGGFTFKRTSGSSVKREEEVLTGNLQTLKIRVHEGYEEFTMVGRRATAILRKATRRLIQLIVSGRDEQSIAEAIIV |
| | AMVFSQEDCMIKAVRGDLNFVNRANQRLNPMHQLLRHFQKDAKVLFQNWGIEPIDNVMGMIGILPDMTPSTEMSLRGIRV |
| | SKMGVDEYSSTERVVVSIDRFLRVRDQRGNVLLSPEEVSETQGTEKLAITYSSSMMWEISGPESVLVNTYQWIIRNWESL |
| | KIQWSQDPTMLYNKMEFEPFQSLVPKATRSRYSGFVRTLFQQMRDVIGTFDTVQIIKLLPFAAAPPEQSRMQFSSLTVNV |
| | RGSGLRILVRGNSPVFNYNKATKRLTVLGKDAGELTEDPDEGTAGVESAVLRGFLILGKEDKRYGPALSINELSNLAKGE |
| | KANVLIGQGDVVLVMKRKRDSSILTDSQTATKRIRMAIN |
| SEQ ID NO: | Amino acid sequence of PB1 protein of A/Swine/Missouri/2124514/2006 |
| 26 | MDVNPTLLFLKVPAQNAISTTFPYTGDPPYSHGTGTGYTMDTVNRTHQYSEKGKWTTNTETGAPQLNPIDGPLPEDNEPS |
| | GYAQTDCVLEAMAFLEESHPGIFENSCLETMEVVQQTRVDKLTQGRQTYDWTLNRNQPAATALANTIEVFRSNSLTANES |
| | GRLINFLKDVMESMDREEVEITTHFRRKRRVRDNMTKKMVTQRTIGKKKQRLSKRSYLIRALTLNTMTKDAERGKLKRRA |
| | IATPGMQIRGFVYFVETLARSICEKLEQSGLPVGGNEKKAKLANVVRKMMTNSQDTELSFTITGDNTKWNENQNPRMFLA |
| | MITYITRNQPEWFRNILSMAPIMFSNKMARLGKGYMFESKRMKLRTQIPAEMLASIDLKYFNESTRKKIEKIRPLLIDGT |
| | ASLSPGMMMGMFNMLSTVLGVSILNLGQKKYTRTTYWWDGLQSSDDFALIVNAPNYEGIQAGVNRFYRTCKLVGINMSKK |
| | KSYINKTGTFEFTSFFYRYGFVANFSMELPSFGVSGINESADMSIGVTVIKNNMINNDLGPATAQMALQLFIKDYRYTYR |
| | CHRGDTQIQTRRSFELKKLWDQTRSNAGLLVSDGGPNLYNIRNLHIPEVCLKWELMDEDYRGRLCNPLNPFVSHKEIDSV |
| | NNAVVMPAHGPAKSMEYDAVATTHSWIPKRNRSILNTSQRGILEDEQMYQKCCNLFEKFFPSSSYRRPVGISSMVEAMVS |
| | RARIDARIDFESGRIKKEEFSEIIRICSTIEELRRQT |
| SEQ ID NO: | Amino acid sequence of PA protein of A/Swine/Missouri/2124514/2006 |
| 27 | MEDFVRQCFNPMIVELAEKAMKEYGEDPKIETNKFAAICTHLEVCFMYSDFHFIDERGESIIVESGDPNALLKHRFEIIE |
| | GRDRTMAWTVVNSICNTTGVEKPKFLPDLYDYKENRFIEIGVTRREVHIYYLEKANKIKSEKTHIHIFSFTGEEMATKAD |
| | YTLDEESRARIKTRLFTIRQEMASRGLWDSFRQSERGEETIEERFEITGTMRRLADQSLPPNFSSLENFRAYVDGFEPNG |
| | CIEGKLSQMSKEVNARIEPFLKTTPRPLKLPDGPPCSQRSKFLLMDALKLSIEDPSHEGEGIPLYDAIKCMKTFFGWKEP |
| | NIIKPHEKGINPNYFLAWKQVLAELQDLENEEKIPKTKNMKKTSQLKWALGENMAPEKVDFEDCKDIGDLKQYDSDEPEP |
| | RSLASWIQSEFNKACELTDSSWIELDEIGEDVAPIEHIASMRRNYFTAEVSHCRATEYIMKGVYINTALLNASCAAMDDF |
| | QLIPMISKCRTKEGRRKTNLYGFIIKGRSHLRNDTDVVNFVSMEFSLTDPRLEPHKWEKYCVLEIGDMLLRTAIGQVSRP |
| | MFLYIRTNGTSKIKMKWGMEMRRCLLQSLQQIESMIEAESSVKEKDMTKEFFENKSETWPIGESPRGVEEGSIGKVCRTL |
| | LAKSVFNSLYASPQLEGFSAESRKLLLIVQALRDNLEPGTFDLGGLYEAIEECLINDPWVLLNASWFNSFLTHALK |
| SEQ ID NO: | Amino acid sequence of HA protein of A/Swine/Missouri/2124514/2006 |
| 28 | MTITFLILLFTVVKGDQICIGYHANNSTEKVDTILERNVTVTHAKNILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVSGNPSFFRN |
| | MVWLTKKGSNYPIAKRSYNNTSGEQMLVIWGIHHPNDDAEQRTLYQNVGTYVSVGTSTLNKRSIPEIATRPKVNGLGGRM |
| | EFSWTLLETWDVINFESTGNLIAPEYGFKISKRGSSGIMKTEKILENCETKCQTPLGAINTTLPFHNIHPLTIGECPKYV |
| | KSDRLILATGLRNVPQIESRGLFGAIAGFIEGGWQGMVDGWYGYHHSNDQGSGYAADKESTQKAIDGITNKVNSVIEKMN |
| | TQFEAVGKEFNNLERRLENLNKKMEDGFIDVWTYNAELLVLMENERTLDFHDSNVKNLYDKVRMQLRDNAKEIGNGCFEF |
| | YHKCDDECMNSVRNGTYDYIKYEEESKLNRNEIKGVKLSNMGVYQILAIYATVAGSLSLAIMIAGISFWMCSNGSLQCRI |
| | CI |
| SEQ ID NO: | Amino acid sequence of NP protein of A/Swine/Missouri/2124514/2006 |
| 29 | MASQGTKRSYEQMETGGERQDATEIRASVGRMIGGIGKFYIQMCTELKLSDYEGRLIQNSMTIERMVLSAFDERRNKYLE |
| | EHPSAGKDPKKTGGPIYRRVDGKWMRELILYDKEEIRRVWRQANNGEDATAGLAHIMIWHSNLNDATYQRTRALVRTGMD |
| | PRMCSLMQGSTLPRRSGAAGAAVKGVGTIAMELIRMIKRGINDRNFWRGENGRKTRIAYERMCNILKGKFQTAAQRAMMD |
| | QVRESRNPGNAEIEDLIFLARSALILRGSVAHKSCLPACVYGLAVASGHDFEREGYSLVGIDPFKLLQNSQVFSLIRPNE |
| | NPAHKSQLVWMACHSAAFEDLRVSSFIRGKKVIPRGRLSTRGVQIASNENVEAMDSNTLELRSRYWAIRTRSGGNTNQQK |
| | ASAGQISVQPTFSVQRNLPFERATVMAAFSGNNEGRTSDMRTEVIRMMENAKPEDLSFQGRGVFELSDEKATSPIVPSFD |
| | MSNEGSYFFGDNAEEYDS |
| SEQ ID NO: | Amino acid sequence of NA protein of A/Swine/Missouri/2124514/2006 |
| 30 | MNPNQKIITIGVVNTTLSTIALLIGVGNLVFNTVIHEKIGDHQIVTYPIITTPAVPNCSDTIITYNNTVINNITTTIITE |
| | EERPFKSPLPLCPFRGFFPFHKDNAIRLGENKDVIVTREPYVSCDNDNCWSFALAQGALLGTKHSNGTIKDRTPYRSLIR |
| | FPIGTAPVLGNYKEICIAWSSSSCFDGKEWMHVCMTGNDNDASAQIIYGGRMTDSIKSWRKDILRTQESECQCIDGTCVV |
| | AVTDGPAANSADYRVYWIREGKIIKYENVPKTKIQHLEECSCYVDIDVYCICRDNWKGSNRPWMRINNETILETGYVCSK |
| | FHSDTPRPADPSTMSCDSPSNVNGGPGVKGFGFKAGDDVWLGRTVSTSGRSGFEIIKVTEGWINSPNHVKSITQTLVSNN |
| | DWSGYSGSFIVKAKDCFQPCFYVELIRGRPNKNDDVSWTSNSIVTFCGLDNEPGSG |
| | NWPDGSNIGFMPK |
| SEQ ID NO: | Amino acid sequence of M1 protein of A/Swine/Missouri/2124514/2006 |
| 31 | MSLLTEVETYVLSIVPSGPLKAEIAQRLEDVFAGKNTDLEALMEWLKTRPILSPLTKGILGFVFTLTVPSERGLQRRRFV |
| | QNALNGNGDPNNMDKAVKLYRKLKREITFHGAKEVALSYSAGALASCMGLIYNRMGTVTTEVAFGLVCATCEQIADSQHR |
| | SHRQMVTTTNPLIRHENRMVIASTTAKAMEQMAGSSEQAAEAMEVASQARQMVQAMRTIGTHPSSSTGLKDDLLENLQAY |
| | QKRMGVQMQRFK |
| SEQ ID NO: | Amino acid sequence of M2 protein of A/Swine/Missouri/2124514/2006 |
| 32 | MSLLTEVETPIRSGWECKCNDSNDPLIDAASIIGILHLILWILDRLFFKCIYRRFKYGLQRGPSTEGVPESMREEYRQKQ |
| | QSAVDVDDGHFVNIVLE |
| SEQ ID NO: | Amino acid sequence of NS1 protein of A/Swine/Missouri/2124514/2006 |
| 33 | MDSTTVSSFQVDCFLWHIRKRFADNGLGDAPFLDRLRRDQKSLKGRGNTLGLDIETATLVGKQIVEWILKEESSDTLKMT |
| | IASVPTSRYLADMTLEEMSRDWLMLMPRQKIIGPLCVRMDQAIMEKNIILKANFSVIFNRLETLILLRAFTEEGTIVGEI |
| | SQLPSLPGHTNEDVKNAIGVLIGGLEWNGNTVRGSENLQRFAWRNRNEDGRPSLPPEKK |
| SEQ ID NO: | Amino acid sequence of NEP protein of A/Swine/Missouri/2124514/2006 |
| 34 | MDSTTVSSFQDILMRMSKMQLGSSSEDLNGMVTRFEALKIYRDSLGETVMRMGDLHYLQRRNEKWREQLGQKFEEIRWLI |
| | EEVRHRLKTTENSFEQITFMQALQLLLEVEQEIRTFSFQLI |
| SEQ ID NO: | Nucleotide sequence of HA viral segment of HS Sys 14 rescue isolate of |
| 35 | A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACGTCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGTCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAAGGTAAATTATGTGATTGAAAAGATGAACACTCAGTTTGAGGCTGTTGGGAAAGAGTTCAACAATCTAGAGAGAGGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTTCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA protein of HS Sys 14 rescue isolate of |
| 36 | A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNKVNYVIEKMN |
| | TQFEAVGKEFNNLERGLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of HS Sys 15 rescue isolate of |
| 37 | A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACGTCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGTCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAAGGTAAATTCTGTGATTGAAAAGATGAACACTCAGTTTGAGACTGTTGGGAAAGAGTTCAACAATCTAGAGAGAAGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTTCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA protein of HS Sys 15 rescue isolate of |
| 38 | A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNKVNSVIEKMN |
| | TQFETVGKEFNNLERRLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of GDE 80.4A rescue isolate of |
| 39 | A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACGTCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGCCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGCCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAAGGTAAATTCTGTGATTGAAAAGATGAACACTCAGTTTGAGGCTGTTGGGAAAGAGTTCAACAATCTAGAGAGAAGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTTCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA protein of GDE 80.4A rescue isolate of |
| 40 | A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKANGQGGRM |
| | EFSWTLLETWDVINFESTGNLIAPEYGFKISKRGSSGIMKTEKTLENCETRCQTPLGAINTTLPFHNIHPLTIGECPKYA |
| | KSDRLVLATGLRNVPQIESRGLFGAIAGFIEGGWQGMVDGWYGYHHSNDQGSGYAADKESTQKAIDGITNKVNSVIEKMN |
| | TQFEAVGKEFNNLERRLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of GDE 80.4B rescue isolate of |
| 41 | A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACATCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGTCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAAGGTAAATTCTGTGATTGAAAAGATGAACACTCAGTTTGAGGCTGTTGGGAAAGAGTTCAACAATCTAGAGAGAAGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTTCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA protein of GDE 80.4B rescue isolate of |
| 42 | A/Chicken/Ohio/494832/2007 |
| | MTITFLILLFTVVKGDQICIGYHANNSTEKVDTILERNITVTHAKDILEKTHNGKLCRLSGIPPLELGDCSIAGWLLGNP |
| | ECDRLLSVPEWSYIVEKENPVNGLCYPGSENDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNKVNSVIEKMN |
| | TQFEAVGKEFNNLERRLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of GDE-80.7B rescue isolate of |
| 43 | A/Chicken/Ohio/494832/2007 |
| | AGCAAAAGCAGGGGTTATACCATAGACAACCGAACAAAGACAATGACCATCACTTTTCTCATCCTCCTGTTCACAGTAGT |
| | GAAAGGGGACCAAATATGTATCGGATACCATGCCAACAATTCCACAGAAAAAGTTGACACAATCTTGGAACGAAACGTCA |
| | CCGTGACTCATGCCAAGGACATTCTTGAAAAGACGCATAATGGAAAGTTGTGCAGATTAAGCGGGATCCCTCCATTGGAA |
| | CTGGGGGATTGCAGCATTGCAGGTTGGCTCCTTGGAAATCCAGAGTGTGACCGGCTCTTAAGTGTACCTGAATGGTCCTA |
| | TATAGTGGAAAAGGAAAACCCGGTGAATGGTCTGTGCTACCCAGGCAGTTTCAATGACTATGAGGAATTGAAACATCTCC |
| | TCACCAGTGTGACACACTTTGAGAAAGTTAAGATCCTGCCTAGAGATCAATGGACCCAGCACACAACAACTGGTGGTTCT |
| | CGGGCCTGTGCAGTATTTGGGAATCCATCATTCTTCAGAAACATGGTCTGGCTTACAAAGAAGGGGTCAAACTACCCAAT |
| | CGCTAAAAGGTCATACAACAACACAAGTGGGGAGCAAATGCTGATAATCTGGGGGGTACATCATCCCAATGACGATGCAG |
| | AACAAAGGACACTGTACCAGAATGTGGGAACATATGTTTCCGTTGGAACATCAACACTAAATAAGAGGTCAATCCCTGAA |
| | ATAGCGACAAGGCCCAAAGTCAATGGACAAGGAGGGAGAATGGAATTCTCTTGGACTCTATTAGAGACATGGGATGTCAT |
| | AAATTTTGAGAGCACTGGTAATTTAATTGCACCAGAATATGGATTCAAAATATCAAAGAGAGGAAGCTCAGGAATTATGA |
| | AGACAGAGAAAACACTTGAAAATTGTGAGACCAGATGCCAGACCCCCTTAGGGGCAATAAATACAACATTGCCCTTTCAC |
| | AACATTCACCCATTGACAATAGGTGAGTGCCCCAAGTATGTCAAGTCAGATAGACTGGTCTTGGCGACAGGACTAAGAAA |
| | TGTTCCTCAGATTGAATCAAGGGGATTGTTTGGAGCAATAGCTGGGTTTATAGAAGGCGGATGGCAAGGGATGGTTGATG |
| | GCTGGTATGGGTATCATCACAGCAATGATCAAGGATCAGGATATGCAGCAGACAAAGAATCCACTCAAAAGGCAATTGAC |
| | GGGATAACTAACAAGGTAAATTCTGTGATTGAAAAGATGAACACTCAGTTTGAGGCTGTTGGGAAAGAGTTCAACAATCT |
| | AGAGAGAAGACTAGAAAACTTAAATAAGAAAATGGAAGATGGATTTCTCGATGTATGGACATACAATGCCGAACTCCTAG |
| | TTCTAATGGAAAATGAGAGGACACTTGATTCCCATGACTCTAATGTGAGGAATCTGTACGATAAGGTCAGAATGCAACTG |
| | AGGGACAATGCTAAGGAAATAGGGAACGGATGCTTTGAGTTTTATCATAAATGTGATGATGAATGCATGAACAGTGTCAG |
| | GAATGGAACATATGATTATCCCAAATATGAAGAAGAGTCCAAGCTGAACAGGAACGAAATCAAAGGGGTGAAATTGAGCG |
| | ATATGGGGGTCTATCAAATACTTGCTATATACGCTACAGTTGCAGGCTCCTTGTCACTGGCAATCATGATAGCTGGGATT |
| | TCTTTCTGGATGTGTTCTAATGGGTCTCTGCAATGCAGAATTTGCATTTGACTGTGAGTCAATTTGTAATTAAAAACACC |
| | CTTGTTTCTACT |
| SEQ ID NO: | Amino acid sequence of HA protein of GDE-80.7B rescue isolate of |
| 44 | A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNKVNSVIEKMN |
| | TQFEAVGKEFNNLERRLENLNKKMEDGFLDVWTYNAELLVLMENERTLDSHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of WT passaged isolate of |
| 45 | A/Chicken/Ohio/494832/2007 |
| | AGCAAAAGCAGGGGTTATACCATAGACAACCGAACAAAGACAATGACCATCACTTTTCTCATCCTCCTGTTCACAGTAGT |
| | GAAAGGGGACCAAATATGTATCGGATACCATGCCAACAATTCCACAGAAAAAGTTGACACAATCTTGGAACGAAACGTCA |
| | CCGTGACTCATGCCAAGGACATTCTTGAAAAGACGCATAATGGAAAGTTGTGCAGATTAAGCGGGATCCCTCCATTGGAA |
| | CTGGGGGATTGCAGCATTGCAGGTTGGCTCCTTGGAAATCCAGAGTGTGACCGGCTCTTAAGTGTACCTGAATGGTCCTA |
| | TATAGTGGAAAAGGAAAACCCGGTGAATGGTCTGTGCTACCCAGGCAGTTTCAATGACTATGAGGAATTGAAACATCTCC |
| | TCACCAGTGTGACACACTTTGAGAAAATTAAGATCCTGCCTAGAGATCAATGGACCCAGCACACAACAACTGGTGGTTCT |
| | CGGGCCTGTGCAGTATTTGGGAATCCATCATTCTTCAGAAACATGGTCTGGCTTACAAAGAAGGGGTCAAACTACCCAAT |
| | CGCTAAAAGGTCATACAACAACACAAGTGGGGAGCAAATGCTGATAATCTGGGGGGTACATCATCCCAATGACGATGCAG |
| | AACAAAGGACACTGTACCAGAATGTGGGAACATATGTTTCCGTTGGAACATCAACACTAAATAAGAGGTCAATCCCTGAA |
| | ATAGCGACAAGGCCCAAAGTCAATGGACAAGGAGGGAGAATGGAATTCTCTTGGACTCTATTAGAGACATGGGATGTCAT |
| | AAATTTTGAGAGCACTGGTAATTTAATTGCACCAGAATATGGATTCAAAATATCAAAGAGAGGAAGCTCAGGAATTATGA |
| | AGACAGAGAAAACACTTGAAAATTGTGAGACCAGATGCCAGACCCCCTTAGGGGCAATAAATACAACATTGCCCTTTCAC |
| | AACATTCACCCATTGACAATAGGTGAGTGCCCCAAGTATATCAAGTCAGATAGACTGGTCTTGGCGACAGGACTAAGAAA |
| | TGTTCCTCAGATTGAATCAAGGGGATTGTTTGGAGCAATAGCTGGGTTTATAGAAGGCGGATGGCAAGGGATGGTTGATG |
| | GCTGGTATGGGTATCATCACAGCAATGATCAAGGATCAGGATATGCAGCAGACAAAGAATCCACTCAAAAGGCAATTGAC |
| | GGGATAACTATCAGGGTAAATTCTGTGATTGAAAAGATGAACACTCAGTTTGAGGCTGTTGGGAAAGAGTTCAACAATCT |
| | AGAGAGAAGACTAGAAAACTTAAATAAGAAAATGGAAGATGGATTTCTCGATGTATGGACATACAATGCCGAACTCCTAG |
| | TTCTAATGGAAAATGAGAGGACACTTGATTTCCATGACTCTAATGTGAGGAATCTGTACGATAAGGTCAGAATGCAACTG |
| | AGGGACAATGCTAAGGAAATAGGGAACGGATGCTTTGAGTTTTATCATAAATGTGATGATGAATGCATGAACAGTGTCAG |
| | GAATGGAACATATGATTATCCCAAATATGAAGAAGAGTCCAAGCTGAACAGGAACGAAATCAAAGGGGTGAAATTGAGCG |
| | ATATGGGGGTCTATCAAATACTTGCTATATACGCTACAGTTGCAGGCTCCTTGTCACTGGCAATCATGATAGCTGGGATT |
| | TCTTTCTGGATGTGTTCTAATGGGTCTCTGCAATGCAGAATTTGCATTTGACTGTGAGTCAATTTGTAATTAAAAACACC |
| | CTTGTTTCTACT |
| SEQ ID NO: | Amino acid sequence of HA protein of WT passaged isolate of |
| 46 | A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKIKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYI |
| | KSDRLVLATGLRNVPQIESRGLFGAIAGFIEGGWQGMVDGWYGYHHSNDQGSGYAADKESTQKAIDGITIRVNSVIEKMN |
| | TQFEAVGKEFNNLERRLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of RG4 P2 rescue |
| 47 | isolate of A/Swine/Missouri/2124514/2006 |
| | AGCAAAAGCAGGGGAAAATAAAAGCAACAAAAATGACCATCACTTTTCTCATCCTCCTGTTCACAGTAGTGAAAGGGGAC |
| | CAAATATGCATCGGATACCATGCCAACAATTCCACAGAAAAAGTTGACACAATCTTGGAACGAAACGTCACCGTGACTCA |
| | TGCCAAGAACATTCTTGAAAAGACGCATAATGGAAAGTTGTGCAGATTGAGCGGGATCCCTCCATTGGAACTGGGGGATT |
| | GCAGCATTGCAGGTTGGCTCCTTGGAAATCCGGAATGTGACCGGCTCTTAAGTGTACCTGAATGGTCCTATATAGTGGAA |
| | AAGGAAAACCCGGTGAATGGTCTGTGCTATCCAGGCAGTTTCAATGATTATGAGGAATTGAAACATCTTCTCACCAGTGT |
| | GACACACTTTGAGAAAGTTAAGATTCTGCCCAGAGATCAATGGACCCAGCACACAACAACTGGTGGTTCTCGGGCCTGTG |
| | CAGTATCTGGAAACCCGTCATTCTTTAGGAACATGGTTTGGCTTACAAAGAAAGGGTCAAACTACCCAATTGCTAAAAGG |
| | TCATACAACAACACAAGTGGGGAGCAAATGCTGGTAATATGGGGGATACATCACCCCAATGACGATGCGGAACAGAGGAC |
| | ACTGTACCAGAATGTGGGAACATATGTTTCCGTTGGAACATCAACACTAAATAAGAGGTCAATCCCTGAAATAGCAACAA |
| | GGCCCAAAGTCAATGGACTGGGAGGAAGAATGGAATTCTCTTGGACTCTATTGGAGACATGGGATGTCATAAATTTTGAG |
| | AGCACTGGTAATTTAATTGCACCAGAATACGGATTCAAAATATCAAAGAGAGGAAGCTCAGGAATTATGAAGACAGAGAA |
| | AATACTTGAAAATTGTGAAACCAAATGTCAGACCCCCTTGGGGGCAATAAATACAACATTGCCCTTTCACAACATTCACC |
| | CATTGACAATAGGTGAGTGCCCCAAGTATGTAAAGTCAGATAGACTGATTTTGGCGACAGGACTAAGAAATGTCCCCCAG |
| | ATTGAATCAAGGGGATTGTTTGGAGCAATAGCTGGGTTTATAGAAGGCGGATGGCAAGGGATGGTTGATGGCTGGTATGG |
| | GTACCATCACAGCAATGATCAAGGATCAGGATATGCAGCAGACAAAGAATCCACTCAAGAGGCAATTGATGGGATAACTA |
| | ACAAAGTAAATTCTGTGATTGAAAAGATGAACACTCAGTTTGAGGCTGTTGGGAAAGAGTTCAACAACCTAGAGAGAAGA |
| | CTGGAAAACTTAAATAAAAAGATGGAAGATGGATTTATTGATGTATGGACATATAATGCCGAACTCCTAGTTCTAATGGA |
| | AAATGAGAGGACACTTGATTTCCATGATTCTAATGTGAAGAATCTGTACGATAAGGTCAGAATGCAATTGAGAGACAATG |
| | CTAAGGAAATAGGGAACGGATGCTTTGAGTTTTATCATAAATGTGATGATGAATGCATGAATAGTGTCAGGAATGGGACA |
| | TATGATTATATCAAATATGAGGAAGAGTCCAAGCTGAACAGGAACGAAATCAAAGGAGTGAAATTGAGCAATATGGGGGT |
| | TTATCAAATACTTGCTATATACGCTACAGTTGCAGGCTCTTTGTCACTGGCAATCATGATAGCTGGGATTTCTTTCTGGA |
| | TGTGTTCTAATGGGTCTCTGCAATGCAGAATTTGCATATGACATTAGGATTTCAGAAGCATGAGAAAAAACACCCTTGTT |
| | TCTACT |
| SEQ ID NO: | Amino acid sequence of HA protein of RG4 P2 rescue isolate of |
| 48 | A/Swine/Missouri/2124514/2006 |
| | MTITFLILLFTVVKGDQICIGYHANNSTEKVDTILERNVTVTHAKNILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVSGNPSFFRN |
| | MVWLTKKGSNYPIAKRSYNNTSGEQMLVIWGIHHPNDDAEQRTLYQNVGTYVSVGTSTLNKRSIPEIATRPKVNGLGGRM |
| | EFSWTLLETWDVINFESTGNLIAPEYGFKISKRGSSGIMKTEKILENCETKCQTPLGAINTTLPFHNIHPLTIGECPKYV |
| | KSDRLILATGLRNVPQIESRGLFGAIAGFIEGGWQGMVDGWYGYHHSNDQGSGYAADKESTQEAIDGITNKVNSVIEKMN |
| | TQFEAVGKEFNNLERRLENLNKKMEDGFIDVWTYNAELLVLMENERTLDFHDSNVKNLYDKVRMQLRDNAKEIGNGCFEF |
| | YHKCDDECMNSVRNGTYDYIKYEEESKLNRNEIKGVKLSNMGVYQILAIYATVAGSLSLAIMIAGISFWMCSNGSLQCRI |
| | CI |
| SEQ ID NO: | Nucleotide sequence of HA viral segment of RG5 P2 rescue isolate of |
| 49 | A/Swine/Missouri/2124514/2006 |
| | AGCAAAAGCAGGGGAAAATAAAAGCAACAAAAATGACCATCACTTTTCTCATCCTCCTGTTCACAGTAGTGAAAGGGGAC |
| | CAAATATGCATCGGATACCATGCCAACAATTCCACAGAAAAAGTTGACACAATCTTGGAACGAAACGTCACCGTGACTCA |
| | TGCCAAGAACATTCTTGAAAAGACGCATAATGGAAAGTTGTGCAGATTGAGCGGGATCCCTCCATTGGAACTGGGGGATT |
| | GCAGCATTGCAGGTTGGCTCCTTGGAAATCCGGAATGTGACCGGCTCTTAAGTGTACCTGAATGGTCCTATATAGTGGAA |
| | AAGGAAAACCCGGTGAATGGTCTGTGCTATCCAGGCAGTTTCAATGATTATGAGGAATTGAAACATCTTCTCACCAGTGT |
| | GACACACTTTGAGAAAGTTAAGATTCTGCCCAGAGATCAATGGACCCAGCACACAACAACTGGTGGTTCTCGGGCCTGTG |
| | CAGTATCTGGAAACCCGTCATTCTTTAGGAACATGGTTTGGCTTACAAAGAAAGGGTCAAACTACCCAATTGCTAAAAGG |
| | TCATACAACAACACAAGTGGGGAGCAAATGCTGGTAATATGGGGGATACATCACCCCAATGACGATGCGGAACAGAGGAC |
| | ACTGTACCAGAATGTGGGAACATATGTTTCCGTTGGAACATCAACACCAAATAAAAGGTCAATCCCTGAAATAGCAACAA |
| | GGCCCAAAGTCAATGGACTGGGAGGAAGAATGGAATTCTCTTGGACTCTATTGGAGACATGGGATGTCATAAATTTTGAG |
| | AGCACTGGTAATTTAATTGCACCAGAATACGGATTCAAAATATCAAAGAGAGGAAGCTCAGGAATTATGAAGACAGAGAA |
| | AATACTTGAAAATTGTGAAACCAAATGTCAGACCCCCTTGGGGGCAATAAATACAACATTGCCCTTTCACAACATTCACC |
| | CATTGACAATAGGTGAGTGCCCCAAGTATGTAAAGTCAGATAGACTGATTTTGGCGACAGGACTAAGAAATGTCCCCCAG |
| | ATTGAATCAAGGGGATTGTTTGGAGCAATAGCTGGGTTTATAGAAGGCGGATGGCAAGGGATGGTTGATGGCTGGTATGG |
| | GTACCATCACAGCAATGATCAAGGATCAGGATATGCAGCAGACAAAGAATCCACTCAAAAGGCAATTGATGGGATAACTA |
| | ACAAAGTAAATTCTGTGATTGAAAAGATGAACACTCAGTTTGAGGCTGTTGGGAAAGAGTTCAACAACCTAGAGAGAAGA |
| | CTGGAAAACTTAAATAAAAAGATGGAAGATGGATTTATTGATGTATGGACATATAATGCCGAACTCCTAGTTCTAATGGA |
| | AAATGAGAGGACACTTGATTTCCATGATTCTAATGTGAAGAATCTGTACGATAAGGTCAGAATGCAATTGAGAGACAATG |
| | CTAAGGAAATAGGGAACGGATGCTTTGAGTTTTATCATAAATGTGATGATGAATGCATGAATAGTGTCAGGAATGGGACA |
| | TATGATTATATCAAATATGAGGAAGAGTCCAAGCTGAACAGGAACGAAATCAAAGGAGTGAAATTGAGCAATATGGGGGT |
| | TTATCAAATACTTGCTATATACGCTACAGTTGCAGGCTCTTTGTCACTGGCAATCATGATAGCTGGGATTTCTTTCTGGA |
| | TGTGTTCTAATGGGTCTCTGCAATGCAGAATTTGCATATGACATTAGGATTTCAGAAGCATGAGAAAAAACACCCTTGTT |
| | TCTACT |
| SEQ ID NO: | Amino acid sequence of HA protein of RG5 P2 rescue isolate of |
| 50 | A/Swine/Missouri/2124514/2006 |
| | MTITFLILLFTVVKGDQICIGYHANNSTEKVDTILERNVTVTHAKNILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVSGNPSFFRN |
| | MVWLTKKGSNYPIAKRSYNNTSGEQMLVIWGIHHPNDDAEQRTLYQNVGTYVSVGTSTPNKRSIPEIATRPKVNGLGGRM |
| | EFSWTLLETWDVINFESTGNLIAPEYGFKISKRGSSGIMKTEKILENCETKCQTPLGAINTTLPFHNIHPLTIGECPKYV |
| | KSDRLILATGLRNVPQIESRGLFGAIAGFIEGGWQGMVDGWYGYHHSNDQGSGYAADKESTQKAIDGITNKVNSVIEKMN |
| | TQFEAVGKEFNNLERRLENLNKKMEDGFIDVWTYNAELLVLMENERTLDFHDSNVKNLYDKVRMQLRDNAKEIGNGCFEF |
| | YHKCDDECMNSVRNGTYDYIKYEEESKLNRNEIKGVKLSNMGVYQILAIYATVAGSLSLAIMIAGISFWMCSNGSLQCRI |
| | CI |
| SEQ ID NO: | Nucleotide sequence of HA viral segment of GDE 80.4A passage |
| 51 | 3 replicate A isolate of A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACGTCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGTCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAACGTAAATTCTGTGATTGAAAAGATGAACACTCAGTTTGAGGCTGTTGGGAAAGAGTTCAACAATCTAGAGAGAAGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTCCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA protein of GDE 80.4A passage |
| 52 | 3 replicate A isolate of A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNNVNSVIEKMN |
| | TQFEAVGKEFNNLERRLENLNKKMEDGFLDVWTYNAELLVLMENERTLDSHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of GDE 80.4A passage |
| 53 | 3 replicate B isolate of A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACGTCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGCCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAACGTAAATTCTGTGATTGAAAAGATGAACACTCAGTTTGAGGCTGTTGGGAAAGAGTTCAACAATCTAGAGAGAAGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTCCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA protein of GDE 80.4A passage |
| 54 | 3 replicate B isolate of A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKANGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNNVNSVIEKMN |
| | TQFEAVGKEFNNLERRLENLNKKMEDGFLDVWTYNAELLVLMENERTLDSHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of GDE 80.4B passage |
| 55 | 3 replicate A isolate of A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACATCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGAAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGTCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAAGGTAAATTCTGTGATTGAAAAGATGAACACTCAGTTTGAGGCTGTTGGGAAAGAGTTCAACAATCTAGAGAGAAGA |
| | CTAGAAAACTTAAATAAGAAAATGGAAGATGGATTTCTCAATGTATGGACATACAATGCCGAACTCCTAGTTCTAATGGA |
| | AAATGAGAGGACACTTGATTTCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA protein of GDE 80.4B passage |
| 56 | 3 replicate A isolate of A/Chicken/Ohio/494832/2007 |
| | MTITFLILLFTVVKGDQICIGYHANNSTEKVDTILERNITVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGKQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNKVNSVIEKMN |
| | TQFEAVGKEFNNLERRLENLNKKMEDGFLNVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of GDE 80.4B passage |
| 57 | 3 replicate B isolate of A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACATCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGTCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGACAACTA |
| | ACAAGGCAAATTCTGTGATTGAAAAGATGAACACTCAGTTTGAGGCTGTTGGGAAAGAGTTCAACAATCTAGAGAGAAGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTTCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA protein of GDE 80.4B passage 3 |
| 58 | replicate B isolate of A/Chicken/Ohio/494832/2007 |
| | MTITFLILLFTVVKGDQICIGYHANNSTEKVDTILERNITVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGTTNKANSVIEKMN |
| | TQFEAVGKEFNNLERRLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLOCRI |
| | CI |
| SEQ ID NO: | Nucleotide sequence of HA viral segment of GDE-80.7B passage |
| 59 | 3 replicate A isolate of A/Chicken/Ohio/494832/2007 |
| | AGCAAAAGCAGGGGTTATACCATAGACAACCGAACAAAGACAATGACCATCACTTTTCTCATCCTCCTGTTCACAGTAGT |
| | GAAAGGGGACCAAATATGTATCGGATACCATGCCAACAATTCCACAGAAAAAGTTGACACAATCTTGGAACGAAACATCA |
| | CCGTGACTCATGCCAAGGACATTCTTGAAAAGACGCATAATGGAAAGTTGTGCAGATTAAGCGGGATCCCTCCATTGGAA |
| | CTGGGGGATTGCAGCATTGCAGGTTGGCTCCTTGGAAATCCAGAGTGTGACCGGCTCTTAAGTGTACCTGAATGGTCCTA |
| | TATAGTGGAAAAGGAAAACCCGGTGAATGGTCTGTGCTACCCAGGCAGTTTCAATGACTATGAGGAATTGAAACATCTCC |
| | TCACCAGTGTGACACACTTTGAGAAAGTTAAGATCCTGCCTAGAGATCAATGGACCCAGCACACAACAACTGGTGGTTCT |
| | CGGGCCTGTGCAGTATTTGGGAATCCATCATTCTTCAGAAACATGGTCTGGCTTACAAAGAAGGGGTCAAACTACCCAAT |
| | CGCTAAAAGGTCATACAACAACACAAGTGGGGAGCAAATGCTGATAATCTGGGGGGTACATCATCCCAATGACGATGCAG |
| | AACAAAGGACACTGTACCAGAATGTGGGAACATATGTTTCCGTTGGAACATCAACACTAAATAAGAGGTCAATCCCTGAA |
| | ATAGCGACAAGGCCCAAAGTCAATGGACAAGGAGGGAGAATGGAATTCTCTTGGACTCTATTAGAGACATGGGATGTCAT |
| | AAATTTTGAGAGCACTGGTAATTTAATTGCACCAGAATATGGATTCAAAATATCAAAGAGAGGAAGCTCAGGAATTATGA |
| | AGACAGAGAAAACACTTGAAAATTGTGAGACCAGATGCCAGACCCCCTTAGGGGCAATAAATACAACATTGCCCTTTCAC |
| | AACATTCACCCATTGACAATAGGTGAGTGCCCCAAGTATGTCAAGTCAGATAGACTGGTCTTGGCGACAGGACTAAGAAA |
| | TGTTCCTCAGATTGAATCAAGGGGATTGTTTGGAGCAATAGCTGGGTTTATAGAAGGCGGATGGCAAGGGATGGTTGATG |
| | GCTGGTATGGGTATCATCACAGCAATGATCAAGGATCAGGATATGCAGCAGACAAAGAATCCACTCAAAAGGCAATTGAC |
| | GGGATAACTAACAACGTAAATTCTGTGATTGAAAAGATGAACACTCAGTTTGAGGCTGTTGGGAAAGAGTTCAACAATCT |
| | AGAGAGAAGACTAGAAAACTTAAATAAGAAAATGGAAGATGGATTTCTCAATGTATGGACATACAATGCCGAACTCCTAG |
| | TTCTAATGGAAAATGAGAGGACACTTGATTTCCATGACTCTAATGTGAGGAATCTGTACGATAAGGTCAGAATGCAACTG |
| | AGGGACAATGCTAAGGAAATAGGGAACGGATGCTTTGAGTTTTATCATAAATGTGATGATGAATGCATGAACAGTGTCAG |
| | GAATGGAACATATGATTATCCCAAATATGAAGAAGAGTCCAAGCTGAACAGGAACGAAATCAAAGGGGTGAAATTGAGCG |
| | ATATGGGGGTCTATCAAATACTTGCTATATACGCTACAGTTGCAGGCTCCTTGTCACTGGCAATCATGATAGCTGGGATT |
| | TCTTTCTGGATGTGTTCTAATGGGTCTCTGCAATGCAGAATTTGCATTTGACTGTGAGTCAATTTGTAATTAAAAACACC |
| | CTTGTTTCTACT |
| SEQ ID NO: | Amino acid sequence of HA protein of GDE-80.7B passage 3 |
| 60 | replicate A isolate of A/Chicken/Ohio/494832/2007 |
| | MTITFLILLFTVVKGDQICIGYHANNSTEKVDTILERNITVTHAKDILEKTHNGKLCRLSGIPPLELGDCSIAGWLLGNP |
| | ECDRLLSVPEWSYIVEKENPVNGLCYPGSENDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNNVNSVIEKMN |
| | TQFEAVGKEFNNLERRLENLNKKMEDGFLNVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of HS_Sys_14 passage |
| 61 | 3 replicate A isolate of A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACGTCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGTCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAAGGTAAATTATGTGATTGAAAAGATGAACACTCAGTTTGAGGCTGTTGGGAAAGAGTTCAACAATCTAGAGAGAGGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTTCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA viral segment of HS_Sys_14 passage |
| 62 | 3 replicate A isolate of A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNKVNYVIEKMN |
| | TQFEAVGKEFNNLERGLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of HS_Sys_14 passage |
| 63 | 3 replicate B isolate of A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACGTCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGTCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAAGGTAAATTATGTGATTGAAAAGATGAACACTCAGTTTGAGGCTGTTGGGAAAGAGTTCAACAATCTAGAGAGAGGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTTCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA viral segment of HS_Sys_14 passage |
| 64 | 3 replicate B isolate of A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNKVNYVIEKMN |
| | TQFEAVGKEFNNLERGLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of HS_Sys_15 passage |
| 65 | 3 replicate A isolate of A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACGTCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGTCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAAGGTAAATTCTGTGATTGAAAAGATGAACACTCAGTTTGAGACTGTTGGGAAAGAGTTCAACAATCTAGAGAGAAGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTTCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA viral segment of HS_Sys_15 passage |
| 66 | 3 replicate A isolate of A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNKVNSVIEKMN |
| | TQFETVGKEFNNLERRLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Nucleotide sequence of HA viral segment of HS_Sys_15 passage |
| 67 | 3 replicate B isolate of A/Chicken/Ohio/494832/2007 |
| | AGCAAAAGCAGGGGAAAACAAAAGCAACAAAAATGACCATCACTTTTCTCATCCTCCTGTTCACAGTAGTGAAAGGGGAC |
| | CAAATATGTATCGGATACCATGCCAACAATTCCACAGAAAAAGTTGACACAATCTTGGAACGAAACGTCACCGTGACTCA |
| | TGCCAAGGACATTCTTGAAAAGACGCATAATGGAAAGTTGTGCAGATTAAGCGGGATCCCTCCATTGGAACTGGGGGATT |
| | GCAGCATTGCAGGTTGGCTCCTTGGAAATCCAGAGTGTGACCGGCTCTTAAGTGTACCTGAATGGTCCTATATAGTGGAA |
| | AAGGAAAACCCGGTGAATGGTCTGTGCTACCCAGGCAGTTTCAATGACTATGAGGAATTGAAACATCTCCTCACCAGTGT |
| | GACACACTTTGAGAAAGTTAAGATCCTGCCTAGAGATCAATGGACCCAGCACACAACAACTGGTGGTTCTCGGGCCTGTG |
| | CAGTATTTGGGAATCCATCATTCTTCAGAAACATGGTCTGGCTTACAAAGAAGGGGTCAAACTACCCAATCGCTAAAAGG |
| | TCATACAACAACACAAGTGGGGAGCAAATGCTGATAATCTGGGGGGTACATCATCCCAATGACGATGCAGAACAAAGGAC |
| | ACTGTACCAGAATGTGGGAACATATGTTTCCGTTGGAACATCAACACTAAATAAGAGGTCAATCCCTGAAATAGCGACAA |
| | GGCCCAAAGTCAATGGACAAGGAGGGAGAATGGAATTCTCTTGGACTCTATTAGAGACATGGGATGTCATAAATTTTGAG |
| | AGCACTGGTAATTTAATTGCACCAGAATATGGATTCAAAATATCAAAGAGAGGAAGCTCAGGAATTATGAAGACAGAGAA |
| | AACACTTGAAAATTGTGAGACCAGATGCCAGACCCCCTTAGGGGCAATAAATACAACATTGCCCTTTCACAACATTCACC |
| | CATTGACAATAGGTGAGTGCCCCAAGTATGTCAAGTCAGATAGACTGGTCTTGGCGACAGGACTAAGAAATGTTCCTCAG |
| | ATTGAATCAAGGGGATTGTTTGGAGCAATAGCTGGGTTTATAGAAGGCGGATGGCAAGGGATGGTTGATGGCTGGTATGG |
| | GTATCATCACAGCAATGATCAAGGATCAGGATATGCAGCAGACAAAGAATCCACTCAAAAGGCAATTGACGGGATAACTA |
| | ACAAGGTAAATTCTGTGATTGAAAAGATGAACACTCAGTTTGAGACTGTTGGGAAAGAGTTCAACAATCTAGAGAGAAGA |
| | CTAGAAAACTTAAATAAGAAAATGGAAGATGGATTTCTCGATGTATGGACATACAATGCCGAACTCCTAGTTCTAATGGA |
| | AAATGAGAGGACACTTGATTTCCATGACTCTAATGTGAGGAATCTGTACGATAAGGTCAGAATGCAACTGAGGGACAATG |
| | CTAAGGAAATAGGGAACGGATGCTTTGAGTTTTATCATAAATGTGATGATGAATGCATGAACAGTGTCAGGAATGGAACA |
| | TATGATTATCCCAAATATGAAGAAGAGTCCAAGCTGAACAGGAACGAAATCAAAGGGGTGAAATTGAGCGATATGGGGGT |
| | CTATCAAATACTTGCTATATACGCTACAGTTGCAGGCTCCTTGTCACTGGCAATCATGATAGCTGGGATTTCTTTCTGGA |
| | TGTGTTCTAATGGGTCTCTGCAATGCAGAATTTGCATTTGACATTAGGATTTCAGAATCATGAGAAAAACACCCTTGTTT |
| | CTACT |
| SEQ ID NO: | Amino acid sequence of HA viral segment of HS_Sys_15 passage |
| 68 | 3 replicate B isolate of A/Chicken/Ohio/494832/2007 |
| | MTITFLILLFTVVKGDQICIGYHANNSTEKVDTILERNVTVTHAKDILEKTHNGKLCRLSGIPPLELGDCSIAGWLLGNP |
| | ECDRLLSVPEWSYIVEKENPVNGLCYPGSFNDYEELKHLLTSVTHFEKVKILPRDQWTQHTTTGGSRACAVFGNPSFFRN |
| | MVWLTKKGSNYPIAKRSYNNTSGEQMLIIWGVHHPNDDAEQRTLYQNVGTYVSVGTSTLNKRSIPEIATRPKVNGQGGRM |
| | EFSWTLLETWDVINFESTGNLIAPEYGFKISKRGSSGIMKTEKTLENCETRCQTPLGAINTTLPFHNIHPLTIGECPKYV |
| | KSDRLVLATGLRNVPQIESRGLFGAIAGFIEGGWQGMVDGWYGYHHSNDQGSGYAADKESTQKAIDGITNKVNSVIEKMN |
| | TQFETVGKEFNNLERRLENLNKKMEDGFLDVWTYNAELLVLMENERTLDFHDSNVRNLYDKVRMQLRDNAKEIGNGCFEF |
| | YHKCDDECMNSVRNGTYDYPKYEEESKLNRNEIKGVKLSDMGVYQILAIYATVAGSLSLAIMIAGISFWMCSNGSLQCRI |
| | CI |
| SEQ ID NO: | Amino acid sequence of NS1 protein of A/Chicken/Ohio/494832/2007 |
| 69 | MDSNTVSSFQVDCFLWHVRKRFADQELGDAPFLDRLRRDQKSLRGRGSTLGLDIETATRAGKQIVERVLEEESDEALKMT |
| | IASVPASRYLTDMTLEEMSRDWFMLMPKQKVAGSLCIRMDQAIMDKNIILKANFSVIFDRLETLILLRAFTEEGAIVGEI |
| | SPLPSLPGHTDEDVKNAIGVLIGGLEWNDNTVRVSETLQRFAWRSSNEDGRPPLPPKQKRKMARTIESEV |
| SEQ ID NO: | Amino acid sequence of NEP protein of A/Chicken/Ohio/494832/2007 |
| 70 | MDSNTVSSFQDILMRMSKMQLGSSSEDLNGMITQFESLKLYRDSLGEAVMRMGDLHSLQSRNGKWREQLSQKFEEIRWLI |
| | EEVRHRLKITENSFEQITFMQALQLLLEVEQEIRTFSFQLI |
| SEQ ID NO: | Amino acid sequence of PB1-F2 protein of A/Chicken/Ohio/494832/2007 |
| 71 | MEQEQDTPWTQLTEHINTQKRENGQQTQRLEHPNLTQLMDHCPRTMSQVDMHKRTVSLKQWLSLKSLTQESLKTRVLKRW |
| | KLFNKQEWTN |
| SEQ ID NO: | Amino acid sequence of PB1-F2 protein of A/swine/Missouri/2124514/2006 |
| 72 | MEQEQDTPWTQSTEHINIQKKGSGRQTLRLGHPNSIQLMDHYLRIMNQVGMHRQTVF |

1. A modified haemagglutinin (HA) protein comprising an amino acid sequence wherein one or more amino acid residues of a trimer interface region thereof is modified, wherein one or more of the modified amino acid residues are present in a lower region of a stalk domain and wherein the lower region of the stalk domain comprises amino acid residues at positions 377 to 397, 439 to 452, 26 to 46, and 325 to 335 of a full length HA protein of an influenza H2 subtype.

2. The modified HA protein of claim 1, wherein the modified HA protein is capable of forming an HA trimer when expressed by an influenza virus grown in cells.

3. The modified HA protein of claim 1, wherein the modifications to the one or more amino acid residues of the trimer interface region increase the stability of an HA trimer formed from the modified HA protein relative to an HA trimer formed from a corresponding unmodified HA protein.

4. The modified HA protein of claim 1, wherein the modified HA protein is of an H2, H1, H5, H3, H7, or H9 subtype.

5. The modified HA protein of claim 4, wherein the modified HA protein is of an H2, H1, or H5 subtype.

6. The modified HA protein of claim 4, wherein the modified HA protein is of an H2 subtype.

7. The modified HA protein of claim 6, wherein the one or more amino acid residues that are modified are selected from the group consisting of V39, L219, V233, V320, K383, I388, N390, K391, V392, S394, A405, R416, D430, F450, and any combination thereof, of a full length H2 amino acid sequence.

8. (canceled)

9. The modified HA protein of claim 7, wherein the modifications to the one or more amino acid residues are selected from the group consisting of V39I, L219P, V233A, V320A, V320I, K383E, I388T, N390I, K391R, K391N, V392A, S394Y, A405T, R416G, D430N, F450S and any combination thereof, of a full length H2 amino acid sequence.

10-12. (canceled)

13. The modified HA protein of claim 1, wherein the lower region of the stalk domain comprises amino acid residues at positions 383 to 394, 439 to 452, 31 to 40, and 325 to 335 of the full length HA protein of the influenza H2 subtype.

14. The modified HA protein of claim 1, wherein the lower region of the stalk domain comprises amino acid residues N26 to D46, L325 to P335, D377 to E397, and L439 to D452 of the full length HA protein of the influenza H2 subtype.

15. The modified HA protein of claim 14, wherein the lower region of the stalk domain comprises amino acid residues N26 to D46, D377 to E397, and L439 to D452 of the full length HA protein of the influenza H2 subtype.

16-17. (canceled)

18. The modified HA protein of claim 1, wherein the modified HA protein comprises an amino acid sequence as set forth in any one of SEQ ID NOs: 36, 38, 40, 42, 44, 46, 48, 50, 52, 54, 56, 58, 60, 62, 64, 66, or 68, or a fragment, variant, or derivative thereof.

19-23. (canceled)

24. An isolated nucleic acid comprising a nucleotide sequence encoding a modified hemagglutinin (HA) protein comprising an amino acid sequence wherein one or more amino acid residues of a trimer interface region thereof is modified, wherein one or more of the modified amino acid residues are present in a lower region of a stalk domain and wherein the lower region of the stalk domain comprises amino acid residues at positions 377 to 397, 439 to 452, 26 to 46, and 325 to 335 of a full length HA protein of an influenza H2 subtype, or a nucleotide sequence complementary thereto.

25. The isolated nucleic acid of claim 24, comprising a nucleotide sequence selected from the group consisting of SEQ ID NOs: 35, 37, 39, 41, 43, 45, 47, 49, 51, 53, 55, 57, 59, 61, 63, 65, or 67, or a fragment, variant, or derivative thereof; or a nucleotide sequence complementary thereto.

26-28. (canceled)

29. An isolated influenza virus, comprising a hemagglutinin (HA) viral segment encoding a modified HA protein comprising an amino acid sequence wherein one or more amino acid residues of a trimer interface region thereof are modified, wherein one or more of the modified amino acid residues are present in a lower region of a stalk domain and wherein the lower region of the stalk domain comprises amino acid residues at positions 377 to 397, 439 to 452, 26 to 46, and 325 to 335 of a full length HA protein of an influenza H2 subtype.

30. The isolated influenza virus of claim 29, wherein the modified HA protein is of an H2, H1, H5, H3, H7, or H9 subtype.

31. The isolated influenza virus of claim 29, wherein the HA viral segment comprises a nucleotide sequence selected from the group consisting of SEQ ID NOs 35, 37, 39, 41, 43, 45, 47, 49, 51, 53, 55, 57, 59, 61, 63, 65, or 67, or a fragment, variant, or derivative thereof.

32-48. (canceled)

49. A vaccine composition, wherein the vaccine composition comprises:
(a) an isolated influenza virus comprising a hemagglutinin (HA) viral segment encoding a modified HA protein comprising an amino acid sequence wherein one or more amino acid residues of a trimer interface region thereof are modified, wherein one or more of the modified amino acid residues are present in a lower region of a stalk domain and wherein the lower region of the stalk domain comprises amino acid residues at positions 377 to 397, 439 to 452, 26 to 46, and 325 to 335 of a full length HA protein of an influenza H2 subtype, and a pharmaceutically acceptable carrier, diluent, or excipient; or
(b) a modified HA protein comprising an amino acid sequence wherein one or more amino acid residues of a trimer interface region thereof are modified, wherein one or more of the modified amino acid residues are present in the lower region of the stalk domain and wherein the lower region of the stalk domain comprises amino acid residues at positions 377 to 397, 439 to 452, 26 to 46, and 325 to 335 of the full length HA protein of the influenza H2 subtype, and a pharmaceutically acceptable carrier, diluent, or excipient; or
(c) an isolated nucleic acid comprising a nucleotide sequence encoding a modified HA protein comprising an amino acid sequence wherein one or more amino acid residues of a trimer interface region thereof is modified, wherein one or more of the modified amino acid residues are present in the lower region of the stalk domain and wherein the lower region of the stalk domain comprises amino acid residues at positions 377 to 397, 439 to 452, 26 to 46, and 325 to 335 of the full length HA protein of the influenza H2 subtype, and a pharmaceutically acceptable carrier, diluent, or excipient.

50. A method of eliciting an immune response and/or preventing and/or treating an influenza-associated disease, disorder, or condition in a subject, said method including the step of administering a therapeutically effective amount of: (a) an isolated influenza virus comprising a hemagglutinin (HA) viral segment encoding a modified HA protein comprising an amino acid sequence wherein one or more amino acid residues of a trimer interface region thereof are modified, wherein one or more of the modified amino acid residues are present in a lower region of a stalk domain and wherein the lower region of the stalk domain comprises amino acid residues at positions 377 to 397, 439 to 452, 26 to 46, and 325 to 335 of a full length HA protein of an influenza H2 subtype; (b) the modified HA protein; (c) an isolated nucleic acid encoding the modified HA protein; or (d) a vaccine composition comprising the isolated influenza virus, the modified HA protein, or the isolated nucleic acid encoding the modified HA protein to the subject to thereby elicit the immune response and/or prevent and/or treat the influenza-associated disease, disorder or condition in the subject.

51-56. (canceled)

57. A method of improving the growth, stability and/or manufacture of an influenza virus in cells, said method including the step of modifying the influenza virus to express a modified HA protein comprising an amino acid sequence wherein one or more amino acid residues of a trimer interface region thereof is modified, wherein one or more of the modified amino acid residues are present in a lower region of a stalk domain and wherein the lower region of the stalk domain comprises amino acid residues at positions 377 to 397, 439 to 452, 26 to 46, and 325 to 335 of a full length HA protein of an influenza H2 subtype.

58-76. (canceled)

77. The modified HA protein of claim 1, wherein an influenza virus expressing the modified HA protein is capable of growth in cells.

78. The isolated nucleic acid of claim 24, wherein an influenza virus expressing the modified HA protein is capable of growth in cells.


SEQUENCE LISTING (74 sequences)

SEQ ID NO: 1 - DNA, 2294 bp, Alphainfluenzavirus influenzae
  source 1..2294: mol_type = other DNA
gactaagagatctaatgtcacagtctcgcactcgcgagatactcaccaaaaccactgtggaccacatggccataatcaaaaagtacacatcaggaaggcaagagaagaaccctgcactcaggatgaagtggatgatggcaatgaaatatccaattacagcagacaagagaataatggaaatgattcctgaaagaaatgaacaggggcaaaccctttggagcaaaacaaatgatgccggctcagaccgagtgatggtatcacctctggctgtgacatggtggaataggaatggaccaacaactagtacagttcattatccaaaggtctataaaacttatttcgaaaaagtcaaaaggttgaaacacgggacctttggccctgttcacttcagaaatcaagttaaaataagacggagggttgacataaaccctggtcatgcagacctcagtgccaaagaggcacaggatgtaatcatggaagtcgttttcccaaatgaagtgggagcgagaatactaacatcggagtcacaactgacgataacaagagagaagaaagaagaactccaggactgcaagatcgcccctttgatggttgcgtacatgctagaaagggagttggttcgtaaaacgaggttcctcccagtggccggtggaacaagcagtgtttacattgaggtgctgcatttaactcaggggacatgctgggagcaaatgtacaccccaggaggggaagtgaggaatgatgatgttgaccaaagcttgattatcgctgccaggaacatagtaagaagggcaacagtatcagcagacccactagcatctctattggagatgtgccacagcacacagattggagggataaggatggtagacattcttcggcaaaatccaacagaggaacaagctgtggacatatgcaaagcagcaatgggcttaaggattagctcatctttcagctttggcggattcactttcaaaagaacaagcgggtcgtcagttaagaaagaagaggaagtgcttacaggcaaccttcaaacattaaaaataagagtacatgaggggtatgaagagttcacaatggttgggagaagagcaacagccattctcaggaaggcaaccagaagattgatccagttaatagtaagtgggagagacgagcagtcaattgctgaagcaataattgtggccatggtattttcacaagaggattgcatgatcaaagcagttcgaggtgatttgaactttgtcaatagggcaaaccagcggctaaatccaatgcatcaactcttgagacacttccaaaaagatgcaaaagtgcttttccaaaactggggaattgaacccattgacaatgtaatgggaatgatcgggatactacctgacatgaccccaagtactgagatgtcgctaagagggataagagtcagtaagatgggagtagatgaatactccagcacagagagagtggtagtgagcattgaccgatttttaagagtccgggaccaacgggggaatgtgctattgtcacctgaagaagtcagcgagacacaagggacagaaaagctgacaataacttattcgtcatcaatgatgtgggaaatcaatggtcctgaatcggttttggtcaatacgtatcagtggatcatcagaaattgggaaactgtgaaaattcaatggtcacaagaccccacgatgttatataacaagatggaattcgagccatttcagtctctggtccctaaggcagccagaggtcagtacagtgggttcgtgaggacactgttccagcagatgcgggatgtgcttggaactttcgacactgttcagataataaaacttcttccctttgctgctgctccaccagaacaaagtaggatgcaattctcctccttgactgtaaatgtaaggggatcaggaatgagaatactagtaaggggcaattctccagtgttcaattacaataaggccactaaaaggcttacagttctcggaaaagatgcaggtacactaaccgaagatccagatgaaggcacagccggagtagagtctgctgttttgagaggattcctcatcttgggcaaagaagacaagagatatggcccagcattgagtatcaatgagctgagcaatcttgcaaaaggagagaaggctaatgtgctaattgggcaaggagacgtggtgttggtaatgaaacggaaacgggactctagcatacttactgacagccagacagcgaccaaaagaattcggatggccatcaattagtgtcgaattgtttaaaacgaccttgtttct

SEQ ID NO: 2 - DNA, 2294 bp, Alphainfluenzavirus influenzae
  source 1..2294: mol_type = other DNA
ttttcttgaagttccagcgcaaaatgccataagcaccacattcccatatactggagatcctccatacagccatggaacaggaacaggatacaccatggacacagttaacagaacacatcaatactcagaaaagggaaaatggacaacaaactcagagactggagcaccccaacttaacccaattgatggaccactgcccgaggacaatgagccaagtggatatgcacaaacggactgtgtccttgaagcaatggctttccttgaagagtctcacccaggaatctttgaaaactcgtgtcttgaaacgatggaagttgttcaacaaacaagagtggacaaattgacccaaggtcgtcagacctatgattggacattaaacaggaatcagccggctgcaactgcattagctaatactatagaggtcttcagatcaaacggtctgacagccaatgaatcaggaaggttaatagattttctcaaggatgtgatggaatcaatggataaagaggaaatggaaataacaacgcatttccaaaggaaaagaagagtgagagacaacatgacaaagaaaatggtcacacaaagaacaataggaaagaagaagcagaggctaaacaaaaggaactacttaataagagcattgacactgaacacaatgacaaaagacgccgaaagaggcaaactaaagagaagagcaattgcaacacccggaatgcaaatcagaggatttgtatactttgttgaaacattagcaaggagcatttgtgagaagcttgaacaatccggactcccagttggaggcaatgaaaagaaggctaaactggcaaatgttgtgagaaaaatgatgactaattcacaagacacagaactctctttcacaatcaccggggacaacaccaaatggaatgaaaatcaaaaccctaggatgtttctggcaatgataacatatataacaagaagccaacctgaatggtttaggaatgtcttgagcattgcacctataatgttctcaaataaaatggcaagactagggaagggatacatgttcgaaagtaagagcatgaagcttcgaacacaaataccggcagagatgctagcaagtattgatttgaaatatttcaatgaatcaacaagaaagaaaatagagaagataagacctcttctaatagatggtacagcctcattgagtcccggaatgatgatgggcatgttcaacatgttaagtacagttttgggggtttcgattctaaatctagggcaaaagaggtacaccaagacaacatactggtgggacggacttcaatcctccgatgactttgctctcatagtgaatgctccgaatcatgagggaatacaagcaggagtagacagattctatagaacctgcaagctggtcggaatcaacatgagcaagaagaagtcctacataaacaggacaggaacatttgaattcacaagctttttctatcgttatggatttgtagccaactttagcatggagttgcccagctttagcgtgtctgggatcaatgaatctgcagacatgagcattggagtaacagtgataaagaacaacatgataaacaatgatcttggaccagcaacagctcaaatggctcttcagctgttcatcaaggattacagatacacgtatcggtgtcacaggggggacacacaaattcagacaaggagatcattcgagctgaagaagttgtgggaacagacccgctcaaaagcaggactgctggtctcagatggaggaccaaatctatacaatatccggaatctccacattccggaagtctgcttgaaatgggagctaatggatgaagactaccagggaaggctttgtaaccccctgaatccgtttgtcagccacaaagagatagagtctgtaaacaatgctgtggtgatgccagctcacggcccagccaaaagcatggaatatgatgctgttgctaccacgcactcctggatccctaagaggaatcgctccatcctcaatacaagccagaggggaatccttgaagatgaacagatgtatcaaaagtgctgcaatctgttcgagaaattcttccctagcagttcatacaggagaccggttgggatttcaagcatggtggaggccatggtttctagggcccgaattgatgcgcgaattgacttcgaatctggacggattaagaaagaggaatttgctgagatcatgaagatctgttccaccattgaagagctcagacggcagaaatagtgaatttagcttgtccttcatgaaaaaatgccttgtttctact

SEQ ID NO: 3 - DNA, 2141 bp, Alphainfluenzavirus influenzae
  source 1..2141: mol_type = other DNA
tagcaggtactgatccaaaatggaagattttgtgcgacaatgcttcaatccaatgatcgtcgagcttgcggaaaaggcaatgaaagaatatggggaagatccaaaaatcgagacaaacaaattcgctgcaatatgcacacacttggaagtgtgtttcatgtattcagatttccatttcattgatgaacgaggtgagtcgataatcgtggagtctggcgatccaaatgcactcctaaaacaccggtttgagataatcgaagggagagatcgtactatggcctggacagtagtgaatagtatttgcaacaccacaggggttgaaaaacccaagtttctcccggatttatacgattacaaagagaatcgtttcattgaaattggagtaaccaggagggaagtccatatatactatttggaaaaggccaataaaataaaatctgagaagacacacatccacatcttttcattcactggagaagaaatggctactaaagcagactacactcttgatgaagaaagtagagcaaggatcaaaactagactattcaccataagacaagaaatggccagtagaggcctctgggattcctttcgtcagtccgagagaggcgaagagacaattgaagaaagatttgaaattacaggaaccatgcgcaggctcgccgaccaaagtctcccaccgaacttctccagccttgaaaactttagagcctatgtggatggattcgaaccaaacggctgcattgagggcaaactttctcaaatgtccaaggaagtaaatgctagaattgaaccattcttgaagacaacaccacgccccctgaggttaccggaagggcctccttgttcccagcggtcgaaatttctgctgatggatgctctgaagcttagcattgaagacccaagtcatgagggcgaggggataccgctgtatgatgcaatcaaatgcatgaaaacctttttcggctggaaagagcctaacattgtcaagccacatgaaaagggcataaaccccaattatctcctggcttggaagcaagtgctagctgagctacaggatattgaaaatgaggagaagattccgaaaacgaagaacatgaagaaaacaagccaattgaagtgggcacttggtgaaaacatggcaccagagaaagtggactttgaggactgcaaagatgtaagcgacttgagacagtatgacagcgatgagcctgagcaaagatcactagcaagttggattcagagtgagttcaacaaggcttgtgaattaactgactcaagttggatagaactcgatgagataggagaggatgttgccccaatcgaacacattgcaagcatgaggagaaattactttacagctgaggtgtctcactgcagggcaacagaatatataatgaaaggagtatacataaacacagccttgctcaacgcttcttgcgcagcaatggatgatttccagttgatcccaatgataagcaaatgcagaaccaaagaagggcggcggaaaacaaatctgtatgggttcatcataaagggaaggtctcatttgaggaacgatactgatgtagtgaattttgtgagcatggagttttctcttactgaccctagactagagccacataaatgggagaagtactgtgtccttgaaataggggacatgctcctacgaactgcaataggccaagtgtcgagacccatgtttctatatgtgaggaccaatggaacttccaaaatcaaaatgaaatggggtatggaaatgagacgttgtcttcttcaatcacttcagcaaattgaaagcatgattgaggccgagtcttctgtcaaagaaaaagacatgaccaaagagttctttgaaaacaaatcagagacatggcccattggggaatcacccaaaggagtggaagaaggctccatcggaaaggtgtgcaggactctgctggcaaaatctgtattcaacagtttgtatgcatctccacaactcgagggattttcagctgagtctagaaagctgctactcattgttcaggcacttagggacaacctggaacctggtaccttcgatcttggagggctatatgaagcaattgaggagtgcctgattaatgatccctgggttttgcttaatgcatcttggtcaa

SEQ ID NO: 4 - DNA, 1769 bp, Alphainfluenzavirus influenzae
  source 1..1769: mol_type = other DNA
aaaagcaggggttataccatagacaaccgaacaaagacaatgaccatcacttttctcatcctcctgttcacagtagtgaaaggggaccaaatatgtatcggataccatgccaacaattccacagaaaaagttgacacaatcttggaacgaaacgtcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagtcaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgtcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactaacaaggtaaattctgtgattgaaaagatgaacactcagtttgaggctgttgggaaagagttcaacaatctagagagaagactagaaaacttaaataagaaaatggaagatggatttctcgatgtatggacatacaatgccgaactcctagttctaatggaaaatgagaggacacttgatttccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgactgtgagtcaatttgtaattaaaaacacccttgtttctact

SEQ ID NO: 5 - DNA, 1568 bp, Alphainfluenzavirus influenzae
  source 1..1568: mol_type = other DNA
tagcaaaaggcagggtagataatcactcaccgagtgacatccacatcatggcgtctcaaggcaccaaacgatcttatgagcagatggaaactggtggagaacgccagaatgccactgaaatcagagcatctgttgggagaatggttggtggaatcgggagattctatatacagatgtgcactgaactcaaactcagtgactatgaagggagactaatccaaaacagcatcacaatagagagaatggttctctcagcatttgatgagagaagaaataaatatctggaagagcatcccagtgctgggaaagaccccaagaagactggaggcccaatctacaggaggagagatgggaaatggatgagagaattgatcctatatgacaaagaggagatcagaaggatttggcgtcaagcgaataatggagaagacgcaactgctggtctcacccatttgatgatctggcactccaatctgaatgatgccacctaccagagaccgagagcacttgtgcgtactgggatggatcccaggatgtgttctctgatgcaaggctcaactcttccgaggaggtctggagctgctggagcagcagtaaaaggagttggaacaatggtgatggaattgatccgaatgatcaagcgagggatcaatgatcggaatttctggagaggcgaaaatgggcggaggacaagaattgcttatgaaagaatgtgcaacatcctcaaagggaagtttcaaacagcagcacaacgagcaatgatggaccaggtgagggaaagccggaatcctgggaatgctgaaattgaagatctcatctttcttgcacggtctgctctcattctgagggggtcagtggctcataagtcttgcctgcctgcttgtgtgtatggacttgctgtggccagtggatacgactttgaaagggagggatactccctagtcggaattgatcctttccgtctgctccaaaacagtcaagtctttagtcttatcagaccaaacgaaaatccagcacataaaagtcagctggtatggatggcatgccactctgcagcttttgaagatctgagagtgtcaagcttcatcagaggaacaagggtagtcccaagaggacaactgtccaccagaggagtccagattgcttcaaatgagaacatggagacaatggactccagtactcttgaactgaggagcagatactgggctataaggaccagaagcggaggaaacactaaccagcagagagcatccgcagggcaaatcagcgtacagcccacgttctctgtacagagaaacctcccattcgagagagcaaccattatggcggcatttacaggaaacactgaaggcagaacttcagacatgagaacagaaatcataaggatgatggaaaatgccagacctgaagatgtgtctttccaggggcggggagtcttcgagctctcggacgaaaaggcaacgaacccgatcgtgccttcctttgacatgagtaacgaaggatcttatttcttcggagacaatgcagaggaatatgacaattaaagaaaaatacccttgtttctactg

SEQ ID NO: 6 - DNA, 1449 bp, Alphainfluenzavirus influenzae
  source 1..1449: mol_type = other DNA
ttagcaggacgaagatgaatccgaatcagaagataataacaatcggggtagtgaataccactctgtcaacaatagcccttctcattggagtggggaacttagttttcaacacagtcatacatgagaaaataggagaccatcaaatagtgacccacccaacaataacgacccctgcagtaccgaactgcagtgacactataataacatacaataatactgtgataaacaacataacaacaacaataacaactgaagcagaaaggcctttcaagtccccactaccgctgtgccccttcagaggattcttcccttttcacaaggacaatgcaatacgactgggtgagaataaagacgtcatagtcacaagggagccttatgttagctgcgataatgacaactgctggtcctttgctctcgcacaaggagcattgctagggactaaacatagcaatgggaccattaaagacaggacaccatataggtctctaattcgtttcccaataggaacagctccagtactaggaaattacaaagagatatgcattgcttggtcgagcagcagttgctttgacgggaaagagtggatgcatgtgtgcatgacagggaacgataatgatgcaagtgcccagataatatatggagggagaatgacagactccattaaatcatggagaaaggacatactaagaacccaggagtctgaatgtcaatgcattgacgggacttgtgttgttgctgtcacagatggccctgctgctaatagtgcagatcacagggtttactggatacgggaaggaagaataataaagtatgaaaatgttcccaaaacaaagatacaacatttagaagaatgctcctgctatgtagacattgatgtttactgtatatgtagggacaattggaagggctctaacagaccttggatgagaatcaacaacgagactatactggaaacagggtatgtatgtagtaaattccactcagacacccccaggccagctgatccttcaacaatgtcatgtgactccccaagcaatgtcaatggaggacccggagtgaaggggtttggtttcaaagctggcaatgatgtatggttaggtagaacagtgtcaactagtggtagatcgggctttgaaattatcaaagttacagaagggtggatcaactctcctaaccatgtcaaatcaattacacaaacactagtgtccaacaatgactggtcaggctattcaggtagcttcattgtcaaagccaaggactgttttcagccctgtttttatgttgagcttatacgagggaggcccaacaagaatgatgacgtctcttggacaagtaatagtatagttactttctgtggactagacaatgaacctggatcgggaaattggccagatggttctaacattgggtttatgcccaagtaatagaaaaaagcaccttgtttctact

SEQ ID NO: 7 - DNA, 1015 bp, Alphainfluenzavirus influenzae
  source 1..1015: mol_type = other DNA
gatattgaaagatgagtcttctaaccgaggtcgaaacgtacgttctctctatcgtcccatcaggccccctcaaagccgagatcgcgcagagacttgaagatgtgtttgcagggaagaacaccgatcttgaggcactcatggaatggctaaagacaagaccaatcctgtcacctctgactaaggggattttaggatttgtgttcacgctcaccgtgcccagtgagcgaggactgcagcgtagacgctttgtccaaaatgcccttaatgggaatggggatccaaacaacatggacagagcggtcaaactgtacaggaagcttaaaagggagataacattccatggggcaaaagaagtggcactcagttattctactggtgcacttgccagttgcatgggcctcatatacaacagaatggggactgtgaccactgaagtggcatttggcctagtgtgcgccacgtgtgagcagattgctgattcccagcatcggtctcacagacaaatggtgacaacaacaaacccactaatcaggcacgagaacagaatggtactggccagtaccacggctaaggccatggagcaaatggcagggtcaagtgagcaggcagcagaggctatggaggttgctagtcaggctagacagatggtgcaggcaatgaggaccattgggactcatcctagctccagtgctggtctgaaagatgatcttcttgaaaatttgcaggcctatcagaaacggatgggagtgcagatgcagcgattcaagtgatcctctcgttattgccgcaagtatcattgggatcttgcacttgatattgtggattcttgatcgtcttttcttcaaatgcatttatcgtcgccttaaatacggtttgaaaagagggccttctacggaaggagtgcctgagtctatgagggaagaatatcggcaggaacagcagagtgctgtggatgttgacgatggtcattttgtcaacatagagctggagtaaaaaactaccttgtttctactaa

SEQ ID NO: 8 - DNA, 871 bp, Alphainfluenzavirus influenzae
  source 1..871: mol_type = other DNA
gggtgacaaaaacataatggattccaacactgtgtcaagctttcaggtagactgctttctttggcatgtccgcaaacgatttgcagaccaagaattgggtgatgccccattccttgaccggcttcgccgagaccagaagtctctaagaggaagaggcagcactcttggtctggacatcgaaacggccactcgtgctggaaagcagatagtggagagggttctggaggaagaatccgacgaggcactcaaaatgactattgcctctgtgcctgcttcacgctacctaactgacatgactcttgaagagatgtcaagagactggttcatgctcatgcccaagcaaaaagtggcaggctccctctgtatcagaatggaccaggcgattatggataagaacatcatactgaaggcaaacttcagtgtgatcttcgatcggctggagacactaatactactcagagctttcactgaagagggagcaattgtcggcgaaatttcaccattgccttctcttccaggacatactgatgaggatgtcaaaaatgcaattggggtcctcatcggaggacttgaatggaatgataacacagttcgagtctctgaaactttacagagattcgcttggagaagcagtaatgaggatgggagacctccactccctccaaagcagaaacggaaaatggcgagaacaattgagtcagaagtttgaagaaataaggtggctgattgaagaagtgcgacatagactaaagatcacagaaaatagttttgaacaaataacatttatgcaagccttacaactactgcttgaagtggagcaagagataagaactttctcgtttcagcttatttaatgataaaaaacaccctg

SEQ ID NO: 9 - protein, 753 aa, Alphainfluenzavirus influenzae
LRDLMSQSRTREILTKTTVDHMAIIKKYTSGRQEKNPALRMKWMMAMKYPITADKRIMEMIPERNEQGQTLWSKTNDAGSDRVMVSPLAVTWWNRNGPTTSTVHYPKVYKTYFEKVKRLKHGTFGPVHFRNQVKIRRRVDINPGHADLSAKEAQDVIMEVVFPNEVGARILTSESQLTITREKKEELQDCKIAPLMVAYMLERELVRKTRFLPVAGGTSSVYIEVLHLTQGTCWEQMYTPGGEVRNDDVDQSLIIAARNIVRRATVSADPLASLLEMCHSTQIGGIRMVDILRQNPTEEQAVDICKAAMGLRISSSFSFGGFTFKRTSGSSVKKEEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGIEPIDNVMGMIGILPDMTPSTEMSLRGIRVSKMGVDEYSSTERVVVSIDRFLRVRDQRGNVLLSPEEVSETQGTEKLTITYSSSMMWEINGPESVLVNTYQWIIRNWETVKIQWSQDPTMLYNKMEFEPFQSLVPKAARGQYSGFVRTLFQQMRDVLGTFDTVQIIKLLPFAAAPPEQSRMQFSSLTVNVRGSGMRILVRGNSPVFNYNKATKRLTVLGKDAGTLTEDPDEGTAGVESAVLRGFLILGKEDKRYGPALSINELSNLAKGEKANVLIGQGDVVLVMKRKRDSSILTDSQTATKRIRMAIN

SEQ ID NO: 10 - protein, 749 aa, Alphainfluenzavirus influenzae
FLEVPAQNAISTTFPYTGDPPYSHGTGTGYTMDTVNRTHQYSEKGKWTTNSETGAPQLNPIDGPLPEDNEPSGYAQTDCVLEAMAFLEESHPGIFENSCLETMEVVQQTRVDKLTQGRQTYDWTLNRNQPAATALANTIEVFRSNGLTANESGRLIDFLKDVMESMDKEEMEITTHFQRKRRVRDNMTKKMVTQRTIGKKKQRLNKRNYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYITRSQPEWFRNVLSIAPIMFSNKMARLGKGYMFESKSMKLRTQIPAEMLASIDLKYFNESTRKKIEKIRPLLIDGTASLSPGMMMGMFNMLSTVLGVSILNLGQKRYTKTTYWWDGLQSSDDFALIVNAPNHEGIQAGVDRFYRTCKLVGINMSKKKSYINRTGTFEFTSFFYRYGFVANFSMELPSFSVSGINESADMSIGVTVIKNNMINNDLGPATAQMALQLFIKDYRYTYRCHRGDTQIQTRRSFELKKLWEQTRSKAGLLVSDGGPNLYNIRNLHIPEVCLKWELMDEDYQGRLCNPLNPFVSHKEIESVNNAVVMPAHGPAKSMEYDAVATTHSWIPKRNRSILNTSQRGILEDEQMYQKCCNLFEKFFPSSSYRRPVGISSMVEAMVSRARIDARIDFESGRIKKEEFAEIMKICSTIEELRRQK

SEQ ID NO: 11 - protein, 707 aa, Alphainfluenzavirus influenzae
MEDFVRQCFNPMIVELAEKAMKEYGEDPKIETNKFAAICTHLEVCFMYSDFHFIDERGESIIVESGDPNALLKHRFEIIEGRDRTMAWTVVNSICNTTGVEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRRLADQSLPPNFSSLENFRAYVDGFEPNGCIEGKLSQMSKEVNARIEPFLKTTPRPLRLPEGPPCSQRSKFLLMDALKLSIEDPSHEGEGIPLYDAIKCMKTFFGWKEPNIVKPHEKGINPNYLLAWKQVLAELQDIENEEKIPKTKNMKKTSQLKWALGENMAPEKVDFEDCKDVSDLRQYDSDEPEQRSLASWIQSEFNKACELTDSSWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRTAIGQVSRPMFLYVRTNGTSKIKMKWGMEMRRCLLQSLQQIESMIEAESSVKEKDMTKEFFENKSETWPIGESPKGVEEGSIGKVCRTLLAKSVFNSLYASPQLEGFSAESRKLLLIVQALRDNLEPGTFDLGGLYEAIEECLINDPWVLLNASWS

SEQ ID NO: 12 - protein, 562 aa, Alphainfluenzavirus influenzae
MTITFLILLFTVVKGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETRCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 13 - protein, 498 aa, Alphainfluenzavirus influenzae
MASQGTKRSYEQMETGGERQNATEIRASVGRMVGGIGRFYIQMCTELKLSDYEGRLIQNSITIERMVLSAFDERRNKYLEEHPSAGKDPKKTGGPIYRRRDGKWMRELILYDKEEIRRIWRQANNGEDATAGLTHLMIWHSNLNDATYQRPRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMETMDSSTLELRSRYWAIRTRSGGNTNQQRASAGQISVQPTFSVQRNLPFERATIMAAFTGNTEGRTSDMRTEIIRMMENARPEDVSFQGRGVFELSDEKATNPIVPSFDMSNEGSYFFGDNAEEYDN

SEQ ID NO: 14 - protein, 469 aa, Alphainfluenzavirus influenzae
MNPNQKIITIGVVNTTLSTIALLIGVGNLVFNTVIHEKIGDHQIVTHPTITTPAVPNCSDTIITYNNTVINNITTTITTEAERPFKSPLPLCPFRGFFPFHKDNAIRLGENKDVIVTREPYVSCDNDNCWSFALAQGALLGTKHSNGTIKDRTPYRSLIRFPIGTAPVLGNYKEICIAWSSSSCFDGKEWMHVCMTGNDNDASAQIIYGGRMTDSIKSWRKDILRTQESECQCIDGTCVVAVTDGPAANSADHRVYWIREGRIIKYENVPKTKIQHLEECSCYVDIDVYCICRDNWKGSNRPWMRINNETILETGYVCSKFHSDTPRPADPSTMSCDSPSNVNGGPGVKGFGFKAGNDVWLGRTVSTSGRSGFEIIKVTEGWINSPNHVKSITQTLVSNNDWSGYSGSFIVKAKDCFQPCFYVELIRGRPNKNDDVSWTSNSIVTFCGLDNEPGSGNWPDGSNIGFMPK

SEQ ID NO: 15 - protein, 252 aa, Alphainfluenzavirus influenzae
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMVTTTNPLIRHENRMVLASTTAKAMEQMAGSSEQAAEAMEVASQARQMVQAMRTIGTHPSSSAGLKDDLLENLQAYQKRMGVQMQRFK

SEQ ID NO: 16 - protein, 97 aa, Alphainfluenzavirus influenzae
MSLLTEVETPIRNGWECRCSDSSDPLVIAASIIGILHLILWILDRLFFKCIYRRLKYGLKRGPSTEGVPESMREEYRQEQQSAVDVDDGHFVNIELE

SEQ ID NO: 17 - DNA, 2299 bp, Alphainfluenzavirus influenzae
  source 1..2299: mol_type = other DNA
tatattcaatatggagagaataaaagaactaagagacctaatgtcgcagtcccgcactcgcgaaatactcaccaagaccactgtggaccatatggccataatcaaaaagtacacatcgggaaggcaagagaagaaccctgcactcagaatgaagtggatgatggcaatgaagtacccaatcacagcagacaagagaataatggacatgattccagagagaaatgaacaaggacaaaccctctggagcaaaacaaacgatgctggatcggaccgcgtgatggtatcacccctggctgtaacatggtggaataggaatggcccaacagcaagcacagttcactaccctaaggtatataaaacttatttcgaaaaagtcgaaaggttaaaacatggtacctttggccctgtccacttcaggaatcaagttaaaataagaaggagagttgacacaaaccctggtcacgcagatctcagagccaaggaggcacaggatgtgatcatggaagttgttttcccaaatgaagtgggggcaagaatactgacatcagagtcacagctgacaataacaaaagagaagaaagaagagctccaggattgtaaaattgctcccttaatggtggcatacatgctagaaaaagagttggtccgtaaaacgaggtttctcccggtggctggtggaacaggcagtgtttatattgaagtgctgcatttaactcaggggacatgctgggagcaaatgtacactccaggaggagaagtgagaaatgatgatgttgaccaaagtttgattattgctgctagaaacatagtaagaagagcagcagtgtcagcagacccattagcatctcttttggaaatgtgccacagcacacagattggaggaataaggatggtggacatccttagacagaatccaacggaggaacaagccgtagacatatgcaaggcagcaatggggctgaggattagctcctctttcagctttggtgggttcactttcaaaagaacaagtgggtcatcagttaagagagaagaagaagtgctcacgggcaaccttcaaacactgaaaataagagtacatgaaggatatgaagaattcacaatggtcgggagaagagcaacagctattctcagaaaagcaaccaggagattgatccagttgatagtaagtgggagagacgagcagtcaattgctgaggcaataattgtggccatggtattttcacaagaggactgcatgatcaaggcagttaggggcgatctgaactttgtcaatagggcaaaccagcgactgaatcccatgcatcaactcttgaggcatttccaaaaagatgcaaaagtgcttttccagaattggggaattgaacccatcgacaatgtgatgggaatgatcgggatattgcccgatatgaccccaagcacggagatgtcgctgagagggataagagtcagcaaaatgggagtagatgaatactccagcacggagagagtggtagtgagcattgacagatttttgagggttcgggatcaacgagggaacgtactattgtctcctgaagaggtcagtgagacacaagggacggagaaattggcaataacttattcgtcatcgatgatgtgggagatcagtggccctgagtcagtgctggtcaacacttatcaatggatcataaggaattgggaaagtttgaaaattcaatggtcacaagatcccacgatgttatacaacaaaatggaatttgaaccatttcagtctcttgtccctaaagcaaccagaagtcgttacagtgggttcgtgaggacactgttccagcaaatgcgggatgtgattggaacatttgacactgtccaaataataaaacttctcccctttgctgctgctccaccagaacagagtaggatgcagttttcctcactgactgtgaatgtgagaggatcagggttgaggatactggtaagaggcaattctccagtattcaattacaataaagcaaccaaaaggcttacagttcttggaaaggatgcaggtgaattgaccgaagacccagatgaaggcacagctggagtggagtctgctgtcctgaggggatttctcattttgggcaaagaagacaagagatatggtccagcattaagcatcaatgaactgagcaatcttgcaaaaggagagaaagctaatgtgctaattgggcaaggagacgtagtgttggtaatgaaacggaaacgggactctagcatacttactgacagccagacagcgaccaaaagaattcggatggccatcaattagtgtcgaatt

SEQ ID NO: 18 - DNA, 2339 bp, Alphainfluenzavirus influenzae
  source 1..2339: mol_type = other DNA
ttgaatggatgtcaatccgaccctacttttcctaaaagttccagcgcaaaatgccataagcaccacattcccttatactggagatcctccatacagccatggaacaggaacaggatacaccatggacacagtcaacagaacacatcaatattcagaaaaagggaagtggacgacaaacactgagactggggcaccccaactcaatccaattgatggaccactacctgaggataatgaaccaagtgggtatgcacagacagactgtgttctagaggccatggctttccttgaagaatcccacccagggatatttgagaattcatgccttgaaacaatggaagttgttcaacaaacaagggtggataaactaactcaaggtcgccagacttatgattggacattaaacagaaatcaaccagcagcaactgcattggccaacaccatagaagtttttaggtcgaatagtctaacagccaatgagtcaggaagactaataaatttcctaaaggatgtaatggaatcaatggatagagaagaagtggagataacaacacactttcgaagaaaaaggagagtgagagacaacatgaccaagaagatggtcacacaaagaacaataggaaagaaaaaacaaagattgagtaagagaagttatctaataagagcacttacattgaatacgatgaccaaagatgcagagagaggcaaattaaaaagaagggctatcgcaacacccgggatgcaaattagagggttcgtgtactttgttgagactttagctaggagcatttgcgaaaagcttgaacagtctggactcccagtagggggaaatgaaaagaaggccaaattggcaaatgttgtgagaaaaatgatgactaattcacaagacacagagctttctttcacaatcactggggacaatactaagtggaatgaaaatcaaaatcctcgaatgttcctggcgatgattacatatatcacccgaaatcaacccgagtggttcagaaacatcctgagcatggcacccataatgttttcaaacaaaatggcaagactaggaaaagggtacatgttcgagagtaaaaggatgaagctccgaacacaaataccagcagaaatgctagcaagcattgatctgaagtatttcaatgaatcaacaaggaagaaaattgagaaaataaggcctcttctaatagatggcacagcatcattgagccctggaatgatgatgggcatgttcaacatgctaagtacggttttgggagtctcgatactgaatcttggacaaaagaaatacaccaggacaacatactggtgggatgggctccaatcctccgacgattttgccctcattgtgaatgcaccaaattatgagggaatacaagcaggagtgaatagattctacaggacctgcaagttagtaggaataaacatgagcaaaaagaagtcctatataaataaaacagggacatttgaattcacaagctttttttatcgctatgggtttgtggctaattttagcatggagctgcccagttttggagtgtctggaataaatgaatcagctgatatgagtattggagtaacagtgataaagaacaatatgataaacaatgatcttggacctgcaacagcccagatggcccttcaattgttcatcaaagactacagatacacatataggtgccacagaggagacacacaaattcagacgagaagatcattcgagctaaagaagctatgggatcaaacccgatcaaatgcaggactattagtatctgatggaggaccaaacttatacaatatcaggaatcttcacattcctgaagtctgcttaaaatgggagctaatggatgaggattatcggggaaggctttgtaatcccctgaatccttttgtcagccataaagagattgattctgtaaacaatgctgtggtgatgccagcccatggcccagccaaaagcatggaatatgatgccgttgcaactacacactcctggatccccaagaggaatcgctctattctcaacacaagccaaaggggaattcttgaggatgaacagatgtaccagaagtgctgcaacctgttcgagaaatttttccctagtagttcatacaggagaccggttggaatttctagcatggtggaggccatggtgtctagggcccggattgatgccagaatcgacttcgagtctggacggattaagaaagaagagttctctgagatcataaggatctgttccaccattgaagaactcagacggcaaacatgatgaatttggcttgtccttcatgaaaaaatgccttgtttctactaatacgagacgatataaa

SEQ ID NO: 19 - DNA, 2230 bp, Alphainfluenzavirus influenzae
  source 1..2230: mol_type = other DNA
ccaaaatggaagactttgtgcgacaatgcttcaatccaatgatcgtcgagcttgcggaaaaggcaatgaaggaatatggggaagatccaaaaatcgaaactaacaaattcgcagcaatatgcactcacttggaagtatgtttcatgtattcggatttccacttcattgatgagcggggcgaatcaataattgtggaatctggtgatccaaatgcattactgaagcaccgatttgaaataattgaaggaagagaccgaacaatggcctggacagtggtgaatagcatctgcaacaccacaggggtcgagaagcctaaatttcttccggatctgtatgattacaaggagaaccgattcattgaaattggagtaacacggagagaggttcatatatactacctagagaaagccaacaagataaaatctgagaagacacacattcacatcttttcatttactggagaagaaatggccaccaaagcagactacactcttgatgaagaaagcagggcaagaatcaaaaccaggctattcactataagacaagaaatggccagcaggggtctatgggattcctttcgtcagtctgaaagaggcgaagagacaattgaggaaagatttgaaatcacaggaaccatgcgtaggcttgccgaccaaagtctcccaccgaacttctccagccttgaaaactttagagcctatgtggatggattcgaaccgaacggctgcattgagggcaagctttctcaaatgtcaaaagaagtgaatgccaggattgagccattcctgaagacaacaccacgccctctcaaattacctgatgggcccccttgctctcagcggtcaaaattcttgctgatggatgccttgaaactaagcatcgaagacccgagtcacgagggagagggtataccactatacgatgcaatcaaatgcatgaagacatttttcggctggaaagagcccaacataatcaaaccacatgagaaaggcataaatcccaattactttctggcttggaagcaagtgctagcagagctccaggaccttgaaaatgaagagaagatcccaaagacaaagaacatgaagaaaacaagccaattgaagtgggcgcttggtgagaatatggcaccagaaaaagtggactttgaggattgcaaggacattggcgatctaaagcagtatgatagtgatgagccagagcctagatcgctggcaagctggatccagagtgaattcaataaggcatgtgaattgaccgactcgagctggatagaacttgatgaaataggagaagatgttgctccgattgaacacattgcaagtatgaggaggaactattttacagcagaagtgtcccactgcagggccactgaatacataatgaaaggagtctacataaatacagctctgctcaatgcatcttgtgcagccatggatgacttccagctgattccaatgataagcaaatgtagaacaaaggaaggaagacggaaaacaaacctgtatgggttcatcataaaaggaagatctcatttgaggaatgatactgatgtggtaaactttgtgagcatggaattttctctcactgacccgaggctagaaccacacaaatgggagaagtattgtgttcttgaaataggagatatgctcctgaggactgcaataggccaagtgtcaaggcccatgttcctgtacattagaaccaatgggacctccaagatcaagatgaaatggggtatggaaatgaggcgctgcctccttcaatctcttcaacagattgagagcatgattgaggctgagtcttctgtcaaagaaaaggacatgactaaggaattctttgaaaacaagtcggaaacgtggccaattggagaatcccccagaggagtagaggaaggctctattgggaaagtatgcagaaccttactggcaaagtctgtattcaacagtctgtacgcatctccacaacttgaggggttttcagctgaatcgagaaaattgcttctcattgttcaggcacttagggacaacctggaacctgggaccttcgatcttggggggctatacgaagcaattgaggagtgcctgattaatgatccctgggttttgcttaatgcatcttggttcaactccttcctcacacatgcactgaaatagttgtggcaatgctactatttgctatccatactgtccaaaaaagtaccttgtttctactaatagaagagcgatga

SEQ ID NO: 20 - DNA, 1805 bp, Alphainfluenzavirus influenzae
  source 1..1805: mol_type = other DNA
ttattcgtcttcagggagcaaaagcaggggttataccatagacaaccgaacaaagacaatgaccatcacttttctcatcctcctgttcacagtagtgaaaggggaccaaatatgcatcggataccatgccaacaattccacagaaaaagttgacacaatcttggaacgaaacgtcaccgtgactcatgccaagaacattcttgaaaagacgcataatggaaagttgtgcagattgagcgggatccctccattggaactgggggattgcagcattgcaggttggctccttggaaatccggaatgtgaccggctcttaagtgtacctgaatggtcctatatagtggaaaaggaaaacccggtgaatggtctgtgctatccaggcagtttcaatgattatgaggaattgaaacatcttctcaccagtgtgacacactttgagaaagttaagattctgcccagagatcaatggacccagcacacaacaactggtggttctcgggcctgtgcagtatctggaaacccgtcattctttaggaacatggtttggcttacaaagaaagggtcaaactacccaattgctaaaaggtcatacaacaacacaagtggggagcaaatgctggtaatatgggggatacatcaccccaatgacgatgcggaacagaggacactgtaccagaatgtgggaacatatgtttccgttggaacatcaacactaaataagaggtcaatccctgaaatagcaacaaggcccaaagtcaatggactgggaggaagaatggaattctcttggactctattggagacatgggatgtcataaattttgagagcactggtaatttaattgcaccagaatacggattcaaaatatcaaagagaggaagctcaggaattatgaagacagagaaaatacttgaaaattgtgaaaccaaatgtcagacccccttgggggcaataaatacaacattgccctttcacaacattcacccattgacaataggtgagtgccccaagtatgtaaagtcagatagactgattttggcgacaggactaagaaatgtcccccagattgaatcaaggggattgtttggagcaatagctgggtttatagaaggcggatggcaagggatggttgatggctggtatgggtaccatcacagcaatgatcaaggatcaggatatgcagcagacaaagaatccactcaaaaggcaattgatgggataactaacaaagtaaattctgtgattgaaaagatgaacactcagtttgaggctgttgggaaagagttcaacaacctagagagaagactggaaaacttaaataaaaagatggaagatggatttattgatgtatggacatataatgccgaactcctagttctaatggaaaatgagaggacacttgatttccatgattctaatgtgaagaatctgtacgataaggtcagaatgcaattgagagacaatgctaaggaaatagggaacggatgctttgagttttatcataaatgtgatgatgaatgcatgaatagtgtcaggaatgggacatatgattatatcaaatatgaggaagagtccaagctgaacaggaacgaaatcaaaggagtgaaattgagcaatatgggggtttatcaaatacttgctatatacgctacagttgcaggctctttgtcactggcaatcatgatagctgggatttctttctggatgtgttctaatgggtctctgcaatgcagaatttgcatatgactgtaagtcaatttgtaattaaaaacacccttgtttctactaatacgagacgatataa

SEQ ID NO: 21 - DNA, 1532 bp, Alphainfluenzavirus influenzae
  source 1..1532: mol_type = other DNA
acccatcaatgagtgacatcgaagccatggcgtctcaaggcaccaaacgatcatatgaacaaatggagactggtggggaacgccaggatgccacagaaatcagagcatctgtcggaagaatgattggtggaatcgggaaattctacatccaaatgtgcactgaactcaaactcagtgactatgagggacgactaatccaaaatagcatgacaatagagagaatggtgctctctgcttttgatgagagaagaaataaatacctagaagagcatcccagtgcagggaaggatcctaagaaaactggaggacccatatatagaagagtagacggaaagtggatgagagaactcattctttatgacaaagaagaaataaggagagtttggcgccaagcaaacaatggtgaagatgcaacagctggtcttgctcatatcatgatttggcactccaatctgaatgatgccacgtaccagagaacaagagcgcttgttcgcaccggaatggatcccagaatgtgctctctaatgcaaggttcaacacttcccagaaggtctggggccgcaggtgctgcagtgaaaggagttggaacaatagcaatggaattaatcagaatgatcaaacgtgggatcaatgaccgaaacttctggagaggtgaaaatggacgaaagacaaggattgcatatgaaagaatgtgcaatattctcaagggaaaatttcagacagctgcccagagggcaatgatggatcaagtgagagaaagtcggaaccccggaaacgctgagattgaagatctcattttcctggcacggtcagcacttattctaaggggatcagttgcacataagtcttgcctgcctgcttgtgtgtatgggcttgcagtggcaagtgggcatgactttgaaagggaagggtattcactggtcgggatagacccatttaaattactccaaaacagtcaagtgttcagcttgataagaccaaatgaaaacccagctcacaagagtcaattagtgtggatggcatgccactctgctgcatttgaggatctgagggtatcaagtttcatcagagggaagaaagtgattccaagaggaaggctctccacaagaggggttcagattgcttcaaatgagaatgtggaagccatggattccaataccttagagctgagaagcagatactgggccataaggaccagaagtggaggaaataccaatcaacagaaggcatccgcgggccagatcagtgtgcaacccacattctcagtgcaacggaatctcccttttgaaagagcaaccgttatggcagctttcagcgggaacaatgaaggacggacatccgatatgcgaacagaagttataaggatgatggaaaatgcaaagccagaagatttgtccttccaggggcggggagtcttcgagctctcggacgaaaaagcaacgagcccgatcgtgccttcctttgacatgagtaatgaaggatcttatttcttcggagacaatgcagaggagtatgacagttgaggaaaaata

SEQ ID NO: 22 - DNA, 1440 bp, Alphainfluenzavirus influenzae
  source 1..1440: mol_type = other DNA
cgagatgaatccgaatcagaagataataacaatcggggtagtgaataccactctgtcaacaatagcccttctcattggagtgggaaacttagttttcaacacagtcatacatgagaaaataggagaccatcaaatagtgacctatccaataataacgacccctgcagtaccgaactgcagtgacactataataacatacaataacactgtgataaacaacataacaacaacaataataactgaagaagaaaggcctttcaagtctccactaccgctgtgccccttcagaggattcttcccttttcacaaggacaatgcaatacgactgggtgaaaacaaagacgtcatagtcacaagagagccttatgttagctgcgataatgacaactgctggtcctttgctctcgcacaaggagcattgctagggaccaaacatagcaatgggaccattaaagacaggacaccatataggtctctaattcgtttcccaataggaacagctccagtactaggaaattataaagagatatgcattgcttggtcgagcagcagttgctttgacgggaaagagtggatgcatgtgtgcatgacagggaacgataatgatgcaagtgcccagataatatatggagggagaatgacagactccattaaatcatggagaaaggacatactaagaactcaggagtctgaatgccaatgcattgacgggacttgtgttgttgctgtcacagatggccctgctgctaatagtgcagattacagggtttactggatacgggagggaaaaataataaagtatgaaaatgttcccaaaacaaagatacaacacttagaagaatgttcctgctatgtggacattgatgtttactgtatatgtagggacaattggaagggctctaacagaccttggatgagaatcaacaacgagactatactggaaacagggtatgtatgtagtaaattccactcagacacccccaggccagctgacccttcaacaatgtcatgtgactccccaagcaatgtcaatggaggacccggagtgaaggggtttggtttcaaagctggcgatgatgtatggttaggtagaacagtgtcgactagtggtagatcgggctttgaaattatcaaagttacagaagggtggatcaactctcctaaccatgtcaaatcaattacacaaacactagtgtccaacaatgactggtcaggctattccggtagcttcattgtcaaagccaaggactgttttcagccctgtttttatgttgagcttatacgagggaggcccaacaagaatgatgacgtctcttggacaagtaatagtatagttactttctgtggactagacaatgaacctggatcgggaaattggccagatggttctaacattgggtttatgcccaagtaatagaaaaaagcaccttgtttctacta

SEQ ID NO: 23 - DNA, 989 bp, Alphainfluenzavirus influenzae
  source 1..989: mol_type = other DNA
ttaaagatgagtcttctaaccgaggtcgaaacgtatgttctctctatcgtcccgtcaggccccctcaaagccgagatagcacagagactcgaagacgtttttgcagggaaaaacaccgatcttgaggctctcatggaatggctaaagacaagaccaatcctgtcacctctgactaaggggattttagggtttgtgttcacgctcaccgtgcccagtgagcgaggactgcagcgtagacgttttgttcagaatgccctcaatgggaatggtgacccgaacaacatggacaaggcggtcaaactttacaggaaactaaaaagggaaataacattccatggggccaaagaagtagcgctcagttactctgctggtgcacttgccagttgcatgggcctcatatacaacagaatgggaactgtcaccactgaggttgcctttggtctggtatgcgcaacctgtgaacagattgctgattctcagcatcgatcccatagacaaatggtgacaacaaccaatccactaatcaggcacgagaacagaatggtgatagccagcacaacagctaaagcaatggaacaaatggctggatcaagcgaacaagcagcagaggctatggaggttgccagccaggctagacaaatggtacaggcaatgagaacaattgggactcaccctagttccagcactggtctaaaagatgatcttcttgaaaatttacaggcctatcagaagcggatgggagtgcaaatgcaacgattcaaatgatcctctcattgatgctgcaagcatcattgggattttgcacctgatattgtggattcttgatcgtctttttttcaaatgcatttaccgtcgctttaaatacggtctgcaaagagggccttctacggaaggagtgccggagtccatgagggaagaatatcgacagaaacagcagagtgctgtggatgttgacgatggtcattttgtcaacatagtgctagagtaaa

SEQ ID NO: 24 - DNA, 851 bp, Alphainfluenzavirus influenzae
  source 1..851: mol_type = other DNA
caaaaagacataatggactccacaactgtgtcaagctttcaggtagactgtttcctttggcacatccgcaaacggtttgcagacaatggattgggtgatgccccattccttgatcggctccgccgagatcaaaagtctctaaaaggaagaggcaacacccttggcctcgacattgaaacagccactcttgttgggaaacaaattgtggagtggattttgaaagaagaatccagcgatacacttaagatgactattgcatctgtacctacttcgcgctatttagctgacatgaccctcgaggaaatgtcacgagactggttaatgctcatgcctaggcaaaagataataggccctctttgtgtgcgaatggaccaggcgatcatggaaaagaacatcatactgaaagcgaacttcagtgtgatctttaaccgattagagactttgatactactaagggctttcacagaggagggaacaatcgttggagaaatttcacaattaccttctcttccaggacatactaatgaggatgtcaaaaatgcaattggggtcctcatcggaggacttgaatggaatggtaacacggttcgaggctctgaaaatctacagagattcgcttggagaaaccgtaatgaggatgggagaccttcactacctccagagaagaaatgaaaagtggcgagagcaattgggacaaaaatttgaggaaataaggtggttaattgaagaagtgcggcacagattgaaaacgacagagaatagttttgaacaaataacattcatgcaagccttacaactactgcttgaagtagaacaagagataaggactttctcgtttcagcttatttaat

SEQ ID NO: 25 - protein, 759 aa, Alphainfluenzavirus influenzae
MERIKELRDLMSQSRTREILTKTTVDHMAIIKKYTSGRQEKNPALRMKWMMAMKYPITADKRIMDMIPERNEQGQTLWSKTNDAGSDRVMVSPLAVTWWNRNGPTASTVHYPKVYKTYFEKVERLKHGTFGPVHFRNQVKIRRRVDTNPGHADLRAKEAQDVIMEVVFPNEVGARILTSESQLTITKEKKEELQDCKIAPLMVAYMLEKELVRKTRFLPVAGGTGSVYIEVLHLTQGTCWEQMYTPGGEVRNDDVDQSLIIAARNIVRRAAVSADPLASLLEMCHSTQIGGIRMVDILRQNPTEEQAVDICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGIEPIDNVMGMIGILPDMTPSTEMSLRGIRVSKMGVDEYSSTERVVVSIDRFLRVRDQRGNVLLSPEEVSETQGTEKLAITYSSSMMWEISGPESVLVNTYQWIIRNWESLKIQWSQDPTMLYNKMEFEPFQSLVPKATRSRYSGFVRTLFQQMRDVIGTFDTVQIIKLLPFAAAPPEQSRMQFSSLTVNVRGSGLRILVRGNSPVFNYNKATKRLTVLGKDAGELTEDPDEGTAGVESAVLRGFLILGKEDKRYGPALSINELSNLAKGEKANVLIGQGDVVLVMKRKRDSSILTDSQTATKRIRMAIN

SEQ ID NO: 26 - protein, 757 aa, Alphainfluenzavirus influenzae
MDVNPTLLFLKVPAQNAISTTFPYTGDPPYSHGTGTGYTMDTVNRTHQYSEKGKWTTNTETGAPQLNPIDGPLPEDNEPSGYAQTDCVLEAMAFLEESHPGIFENSCLETMEVVQQTRVDKLTQGRQTYDWTLNRNQPAATALANTIEVFRSNSLTANESGRLINFLKDVMESMDREEVEITTHFRRKRRVRDNMTKKMVTQRTIGKKKQRLSKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYITRNQPEWFRNILSMAPIMFSNKMARLGKGYMFESKRMKLRTQIPAEMLASIDLKYFNESTRKKIEKIRPLLIDGTASLSPGMMMGMFNMLSTVLGVSILNLGQKKYTRTTYWWDGLQSSDDFALIVNAPNYEGIQAGVNRFYRTCKLVGINMSKKKSYINKTGTFEFTSFFYRYGFVANFSMELPSFGVSGINESADMSIGVTVIKNNMINNDLGPATAQMALQLFIKDYRYTYRCHRGDTQIQTRRSFELKKLWDQTRSNAGLLVSDGGPNLYNIRNLHIPEVCLKWELMDEDYRGRLCNPLNPFVSHKEIDSVNNAVVMPAHGPAKSMEYDAVATTHSWIPKRNRSILNTSQRGILEDEQMYQKCCNLFEKFFPSSSYRRPVGISSMVEAMVSRARIDARIDFESGRIKKEEFSEIIRICSTIEELRRQT

SEQ ID NO: 27 - protein, 716 aa, Alphainfluenzavirus influenzae
MEDFVRQCFNPMIVELAEKAMKEYGEDPKIETNKFAAICTHLEVCFMYSDFHFIDERGESIIVESGDPNALLKHRFEIIEGRDRTMAWTVVNSICNTTGVEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRRLADQSLPPNFSSLENFRAYVDGFEPNGCIEGKLSQMSKEVNARIEPFLKTTPRPLKLPDGPPCSQRSKFLLMDALKLSIEDPSHEGEGIPLYDAIKCMKTFFGWKEPNIIKPHEKGINPNYFLAWKQVLAELQDLENEEKIPKTKNMKKTSQLKWALGENMAPEKVDFEDCKDIGDLKQYDSDEPEPRSLASWIQSEFNKACELTDSSWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRTAIGQVSRPMFLYIRTNGTSKIKMKWGMEMRRCLLQSLQQIESMIEAESSVKEKDMTKEFFENKSETWPIGESPRGVEEGSIGKVCRTLLAKSVFNSLYASPQLEGFSAESRKLLLIVQALRDNLEPGTFDLGGLYEAIEECLINDPWVLLNASWFNSFLTHALK

SEQ ID NO: 28 - protein, 562 aa, Alphainfluenzavirus influenzae
MTITFLILLFTVVKGDQICIGYHANNSTEKVDTILERNVTVTHAKNILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVSGNPSFFRNMVWLTKKGSNYPIAKRSYNNTSGEQMLVIWGIHHPNDDAEQRTLYQNVGTYVSVGTSTLNKRSIPEIATRPKVNGLGGRMEFSWTLLETWDVINFESTGNLIAPEYGFKISKRGSSGIMKTEKILENCETKCQTPLGAINTTLPFHNIHPLTIGECPKYVKSDRLILATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFIDVWTYNAELLVLMENERTLDFHDSNVKNLYDKVRMQLRDNAKEIGNGCFEFYHKCDDECMNSVRNGTYDYIKYEEESKLNRNEIKGVKLSNMGVYQILAIYATVAGSLSLAIMIAGISFWMCSNGSLQCRICI

SEQ ID NO: 29 - protein, 498 aa, Alphainfluenzavirus influenzae
MASQGTKRSYEQMETGGERQDATEIRASVGRMIGGIGKFYIQMCTELKLSDYEGRLIQNSMTIERMVLSAFDERRNKYLEEHPSAGKDPKKTGGPIYRRVDGKWMRELILYDKEEIRRVWRQANNGEDATAGLAHIMIWHSNLNDATYQRTRALVRTGMDPRMCSLMQGSTLPRRSGAAGAAVKGVGTIAMELIRMIKRGINDRNFWRGENGRKTRIAYERMCNILKGKFQTAAQRAMMDQVRESRNPGNAEIEDLIFLARSALILRGSVAHKSCLPACVYGLAVASGHDFEREGYSLVGIDPFKLLQNSQVFSLIRPNENPAHKSQLVWMACHSAAFEDLRVSSFIRGKKVIPRGRLSTRGVQIASNENVEAMDSNTLELRSRYWAIRTRSGGNTNQQKASAGQISVQPTFSVQRNLPFERATVMAAFSGNNEGRTSDMRTEVIRMMENAKPEDLSFQGRGVFELSDEKATSPIVPSFDMSNEGSYFFGDNAEEYDS

SEQ ID NO: 30 - protein, 469 aa, Alphainfluenzavirus influenzae
MNPNQKIITIGVVNTTLSTIALLIGVGNLVFNTVIHEKIGDHQIVTYPIITTPAVPNCSDTIITYNNTVINNITTTIITEEERPFKSPLPLCPFRGFFPFHKDNAIRLGENKDVIVTREPYVSCDNDNCWSFALAQGALLGTKHSNGTIKDRTPYRSLIRFPIGTAPVLGNYKEICIAWSSSSCFDGKEWMHVCMTGNDNDASAQIIYGGRMTDSIKSWRKDILRTQESECQCIDGTCVVAVTDGPAANSADYRVYWIREGKIIKYENVPKTKIQHLEECSCYVDIDVYCICRDNWKGSNRPWMRINNETILETGYVCSKFHSDTPRPADPSTMSCDSPSNVNGGPGVKGFGFKAGDDVWLGRTVSTSGRSGFEIIKVTEGWINSPNHVKSITQTLVSNNDWSGYSGSFIVKAKDCFQPCFYVELIRGRPNKNDDVSWTSNSIVTFCGLDNEPGSGNWPDGSNIGFMPK

SEQ ID NO: 31 - protein, 252 aa, Alphainfluenzavirus influenzae
MSLLTEVETYVLSIVPSGPLKAEIAQRLEDVFAGKNTDLEALMEWLKTRPILSPLTKGILGFVFTLTVPSERGLQRRRFVQNALNGNGDPNNMDKAVKLYRKLKREITFHGAKEVALSYSAGALASCMGLIYNRMGTVTTEVAFGLVCATCEQIADSQHRSHRQMVTTTNPLIRHENRMVIASTTAKAMEQMAGSSEQAAEAMEVASQARQMVQAMRTIGTHPSSSTGLKDDLLENLQAYQKRMGVQMQRFK

SEQ ID NO: 32 - protein, 97 aa, Alphainfluenzavirus influenzae
MSLLTEVETPIRSGWECKCNDSNDPLIDAASIIGILHLILWILDRLFFKCIYRRFKYGLQRGPSTEGVPESMREEYRQKQQSAVDVDDGHFVNIVLE

SEQ ID NO: 33 - protein, 219 aa, Alphainfluenzavirus influenzae
MDSTTVSSFQVDCFLWHIRKRFADNGLGDAPFLDRLRRDQKSLKGRGNTLGLDIETATLVGKQIVEWILKEESSDTLKMTIASVPTSRYLADMTLEEMSRDWLMLMPRQKIIGPLCVRMDQAIMEKNIILKANFSVIFNRLETLILLRAFTEEGTIVGEISQLPSLPGHTNEDVKNAIGVLIGGLEWNGNTVRGSENLQRFAWRNRNEDGRPSLPPEKK

SEQ ID NO: 34 - protein, 121 aa, Alphainfluenzavirus influenzae
MDSTTVSSFQDILMRMSKMQLGSSSEDLNGMVTRFEALKIYRDSLGETVMRMGDLHYLQRRNEKWREQLGQKFEEIRWLIEEVRHRLKTTENSFEQITFMQALQLLLEVEQEIRTFSFQLI

SEQ ID NO: 35 - DNA, 1765 bp, synthetic construct
  source 1..1765: mol_type = other DNA
agcaaaagcaggggaaaacaaaagcaacaaaaatgaccatcacttttctcatcctcctgttcacagtagtgaaaggggaccaaatatgtatcggataccatgccaacaattccacagaaaaagttgacacaatcttggaacgaaacgtcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagtcaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgtcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactaacaaggtaaattatgtgattgaaaagatgaacactcagtttgaggctgttgggaaagagttcaacaatctagagagaggactagaaaacttaaataagaaaatggaagatggatttctcgatgtatggacatacaatgccgaactcctagttctaatggaaaatgagaggacacttgatttccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgacattaggatttcagaatcatgagaaaaacacccttgtttctact

SEQ ID NO: 36 - protein, 562 aa, synthetic construct
MTITFLILLFTVVKGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETRCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGITNKVNYVIEKMNTQFEAVGKEFNNLERGLENLNKKMEDGFLDVWTYNAELLVLMENERTLDFHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 37 - DNA, 1765 bp, synthetic construct
  source 1..1765: mol_type = other DNA
agcaaaagcaggggaaaacaaaagcaacaaaaatgaccatcacttttctcatcctcctgttcacagtagtgaaaggggaccaaatatgtatcggataccatgccaacaattccacagaaaaagttgacacaatcttggaacgaaacgtcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagtcaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgtcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactaacaaggtaaattctgtgattgaaaagatgaacactcagtttgagactgttgggaaagagttcaacaatctagagagaagactagaaaacttaaataagaaaatggaagatggatttctcgatgtatggacatacaatgccgaactcctagttctaatggaaaatgagaggacacttgatttccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgacattaggatttcagaatcatgagaaaaacacccttgtttctact

SEQ ID NO: 38 - protein, 562 aa, synthetic construct
MTITFLILLFTVVKGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETRCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGITNKVNSVIEKMNTQFETVGKEFNNLERRLENLNKKMEDGFLDVWTYNAELLVLMENERTLDFHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 39 - DNA, 1765 bp, synthetic construct
  source 1..1765: mol_type = other DNA
agcaaaagcaggggaaaacaaaagcaacaaaaatgaccatcacttttctcatcctcctgttcacagtagtgaaaggggaccaaatatgtatcggataccatgccaacaattccacagaaaaagttgacacaatcttggaacgaaacgtcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagccaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgccaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactaacaaggtaaattctgtgattgaaaagatgaacactcagtttgaggctgttgggaaagagttcaacaatctagagagaagactagaaaacttaaataagaaaatggaagatggatttctcgatgtatggacatacaatgccgaactcctagttctaatggaaaatgagaggacacttgatttccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgacattaggatttcagaatcatgagaaaaacacccttgtttctact

SEQ ID NO: 40 - protein, 562 aa, synthetic construct
MTITFLILLFTVVKGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKANGQGGRMEFSWTLLETWDVINFESTGNLIAPEYGFKISKRGSSGIMKTEKTLENCETRCQTPLGAINTTLPFHNIHPLTIGECPKYAKSDRLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 41 - DNA, 1765 bp, synthetic construct
  source 1..1765: mol_type = other DNA
agcaaaagcaggggaaaacaaaagcaacaaaaatgaccatcacttttctcatcctcctgttcacagtagtgaaaggggaccaaatatgtatcggataccatgccaacaattccacagaaaaagttgacacaatcttggaacgaaacatcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagtcaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgtcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactaacaaggtaaattctgtgattgaaaagatgaacactcagtttgaggctgttgggaaagagttcaacaatctagagagaagactagaaaacttaaataagaaaatggaagatggatttctcgatgtatggacatacaatgccgaactcctagttctaatggaaaatgagaggacacttgatttccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgacattaggatttcagaatcatgagaaaaacacccttgtttctact

SEQ ID NO: 42 - protein, 562 aa, synthetic construct
MTITFLILLFTVVKGDQICIGYHANNSTEKVDTILERNITVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETRCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 43 - DNA, 1772 bp, synthetic construct
  source 1..1772: mol_type = other DNA
agcaaaagcaggggttataccatagacaaccgaacaaagacaatgaccatcacttttctcatcctcctgttcacagtagtgaaaggggaccaaatatgtatcggataccatgccaacaattccacagaaaaagttgacacaatcttggaacgaaacgtcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagtcaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgtcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactaacaaggtaaattctgtgattgaaaagatgaacactcagtttgaggctgttgggaaagagttcaacaatctagagagaagactagaaaacttaaataagaaaatggaagatggatttctcgatgtatggacatacaatgccgaactcctagttctaatggaaaatgagaggacacttgattcccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgactgtgagtcaatttgtaattaaaaacacccttgtttctact

SEQ ID NO: 44 - protein, 562 aa, synthetic construct
MTITFLILLFTVVKGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETRCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDSHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 45 - DNA, 1772 bp, synthetic construct
  source 1..1772: mol_type = other DNA
agcaaaagcaggggttataccatagacaaccgaacaaagacaatgaccatcacttttctcatcctcctgttcacagtagtgaaaggggaccaaatatgtatcggataccatgccaacaattccacagaaaaagttgacacaatcttggaacgaaacgtcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaaattaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagtcaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatatcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactatcagggtaaattctgtgattgaaaagatgaacactcagtttgaggctgttgggaaagagttcaacaatctagagagaagactagaaaacttaaataagaaaatggaagatggatttctcgatgtatggacatacaatgccgaactcctagttctaatggaaaatgagaggacacttgatttccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgactgtgagtcaatttgtaattaaaaacacccttgtttctact

SEQ ID NO: 46 - protein, 562 aa, synthetic construct
MTITFLILLFTVVKGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKIKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETRCQTPLGAINTTLPFHNIHPLTIGECPKYIKSDRLVLATGLRNVPQIESRGLFGAIAGFIEGGWQGMVDGWYGYHHSNDQGSGYAADKESTQKAIDGITIRVNSVIEKMNTQFEAVGKEFNNLERRLENLNKKMEDGFLDVWTYNAELLVLMENERTLDFHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 47 - DNA, 1766 bp, synthetic construct
  source 1..1766: mol_type = other DNA
agcaaaagcaggggaaaataaaagcaacaaaaatgaccatcacttttctcatcctcctgttcacagtagtgaaaggggaccaaatatgcatcggataccatgccaacaattccacagaaaaagttgacacaatcttggaacgaaacgtcaccgtgactcatgccaagaacattcttgaaaagacgcataatggaaagttgtgcagattgagcgggatccctccattggaactgggggattgcagcattgcaggttggctccttggaaatccggaatgtgaccggctcttaagtgtacctgaatggtcctatatagtggaaaaggaaaacccggtgaatggtctgtgctatccaggcagtttcaatgattatgaggaattgaaacatcttctcaccagtgtgacacactttgagaaagttaagattctgcccagagatcaatggacccagcacacaacaactggtggttctcgggcctgtgcagtatctggaaacccgtcattctttaggaacatggtttggcttacaaagaaagggtcaaactacccaattgctaaaaggtcatacaacaacacaagtggggagcaaatgctggtaatatgggggatacatcaccccaatgacgatgcggaacagaggacactgtaccagaatgtgggaacatatgtttccgttggaacatcaacactaaataagaggtcaatccctgaaatagcaacaaggcccaaagtcaatggactgggaggaagaatggaattctcttggactctattggagacatgggatgtcataaattttgagagcactggtaatttaattgcaccagaatacggattcaaaatatcaaagagaggaagctcaggaattatgaagacagagaaaatacttgaaaattgtgaaaccaaatgtcagacccccttgggggcaataaatacaacattgccctttcacaacattcacccattgacaataggtgagtgccccaagtatgtaaagtcagatagactgattttggcgacaggactaagaaatgtcccccagattgaatcaaggggattgtttggagcaatagctgggtttatagaaggcggatggcaagggatggttgatggctggtatgggtaccatcacagcaatgatcaaggatcaggatatgcagcagacaaagaatccactcaagaggcaattgatgggataactaacaaagtaaattctgtgattgaaaagatgaacactcagtttgaggctgttgggaaagagttcaacaacctagagagaagactggaaaacttaaataaaaagatggaagatggatttattgatgtatggacatataatgccgaactcctagttctaatggaaaatgagaggacacttgatttccatgattctaatgtgaagaatctgtacgataaggtcagaatgcaattgagagacaatgctaaggaaatagggaacggatgctttgagttttatcataaatgtgatgatgaatgcatgaatagtgtcaggaatgggacatatgattatatcaaatatgaggaagagtccaagctgaacaggaacgaaatcaaaggagtgaaattgagcaatatgggggtttatcaaatacttgctatatacgctacagttgcaggctctttgtcactggcaatcatgatagctgggatttctttctggatgtgttctaatgggtctctgcaatgcagaatttgcatatgacattaggatttcagaagcatgagaaaaaacacccttgtttctact

SEQ ID NO: 48 - protein, 562 aa, synthetic construct
MTITFLILLFTVVKGDQICIGYHANNSTEKVDTILERNVTVTHAKNILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVSGNPSFFRNMVWLTKKGSNYPIAKRSYNNTSGEQMLVIWGIHHPNDDAEQRTLYQNVGTYVSVGTSTLNKRSIPEIATRPKVNGLGGRMEFSWTLLETWDVINFESTGNLIAPEYGFKISKRGSSGIMKTEKILENCETKCQTPLGAINTTLPFHNIHPLTIGECPKYVKSDRLILATGLRNVPQIESRGLFGAIAGFIEGGWQGMVDGWYGYHHSNDQGSGYAADKESTQEAIDGITNKVNSVIEKMNTQFEAVGKEFNNLERRLENLNKKMEDGFIDVWTYNAELLVLMENERTLDFHDSNVKNLYDKVRMQLRDNAKEIGNGCFEFYHKCDDECMNSVRNGTYDYIKYEEESKLNRNEIKGVKLSNMGVYQILAIYATVAGSLSLAIMIAGISFWMCSNGSLQCRICI

SEQ ID NO: 49 - DNA, 1766 bp, synthetic construct
  source 1..1766: mol_type = other DNA
agcaaaagcaggggaaaataaaagcaacaaaaatgaccatcacttttctcatcctcctgttcacagtagtgaaaggggaccaaatatgcatcggataccatgccaacaattccacagaaaaagttgacacaatcttggaacgaaacgtcaccgtgactcatgccaagaacattcttgaaaagacgcataatggaaagttgtgcagattgagcgggatccctccattggaactgggggattgcagcattgcaggttggctccttggaaatccggaatgtgaccggctcttaagtgtacctgaatggtcctatatagtggaaaaggaaaacccggtgaatggtctgtgctatccaggcagtttcaatgattatgaggaattgaaacatcttctcaccagtgtgacacactttgagaaagttaagattctgcccagagatcaatggacccagcacacaacaactggtggttctcgggcctgtgcagtatctggaaacccgtcattctttaggaacatggtttggcttacaaagaaagggtcaaactacccaattgctaaaaggtcatacaacaacacaagtggggagcaaatgctggtaatatgggggatacatcaccccaatgacgatgcggaacagaggacactgtaccagaatgtgggaacatatgtttccgttggaacatcaacaccaaataaaaggtcaatccctgaaatagcaacaaggcccaaagtcaatggactgggaggaagaatggaattctcttggactctattggagacatgggatgtcataaattttgagagcactggtaatttaattgcaccagaatacggattcaaaatatcaaagagaggaagctcaggaattatgaagacagagaaaatacttgaaaattgtgaaaccaaatgtcagacccccttgggggcaataaatacaacattgccctttcacaacattcacccattgacaataggtgagtgccccaagtatgtaaagtcagatagactgattttggcgacaggactaagaaatgtcccccagattgaatcaaggggattgtttggagcaatagctgggtttatagaaggcggatggcaagggatggttgatggctggtatgggtaccatcacagcaatgatcaaggatcaggatatgcagcagacaaagaatccactcaaaaggcaattgatgggataactaacaaagtaaattctgtgattgaaaagatgaacactcagtttgaggctgttgggaaagagttcaacaacctagagagaagactggaaaacttaaataaaaagatggaagatggatttattgatgtatggacatataatgccgaactcctagttctaatggaaaatgagaggacacttgatttccatgattctaatgtgaagaatctgtacgataaggtcagaatgcaattgagagacaatgctaaggaaatagggaacggatgctttgagttttatcataaatgtgatgatgaatgcatgaatagtgtcaggaatgggacatatgattatatcaaatatgaggaagagtccaagctgaacaggaacgaaatcaaaggagtgaaattgagcaatatgggggtttatcaaatacttgctatatacgctacagttgcaggctctttgtcactggcaatcatgatagctgggatttctttctggatgtgttctaatgggtctctgcaatgcagaatttgcatatgacattaggatttcagaagcatgagaaaaaacacccttgtttctact

SEQ ID NO: 50 - protein, 562 aa, synthetic construct
MTITFLILLFTVVKGDQICIGYHANNSTEKVDTILERNVTVTHAKNILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVSGNPSFFRNMVWLTKKGSNYPIAKRSYNNTSGEQMLVIWGIHHPNDDAEQRTLYQNVGTYVSVGTSTPNKRSIPEIATRPKVNGLGGRMEFSWTLLETWDVINFESTGNLIAPEYGFKISKRGSSGIMKTEKILENCETKCQTPLGAINTTLPFHNIHPLTIGECPKYVKSDRLILATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFIDVWTYNAELLVLMENERTLDFHDSNVKNLYDKVRMQLRDNAKEIGNGCFEFYHKCDDECMNSVRNGTYDYIKYEEESKLNRNEIKGVKLSNMGVYQILAIYATVAGSLSLAIMIAGISFWMCSNGSLQCRICI

SEQ ID NO: 51 - DNA, 1765 bp, synthetic construct
  source 1..1765: mol_type = other DNA
agcaaaagcaggggaaaacaaaagcaacaaaaatgaccatcacttttctcatcctcctgttcacagtagtgaaaggggaccaaatatgtatcggataccatgccaacaattccacagaaaaagttgacacaatcttggaacgaaacgtcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagtcaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgtcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactaacaacgtaaattctgtgattgaaaagatgaacactcagtttgaggctgttgggaaagagttcaacaatctagagagaagactagaaaacttaaataagaaaatggaagatggatttctcgatgtatggacatacaatgccgaactcctagttctaatggaaaatgagaggacacttgattcccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgacattaggatttcagaatcatgagaaaaacacccttgtttctact

SEQ ID NO: 52 - protein, 562 aa, synthetic construct
MTITFLILLFTVVKGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETRCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGITNNVNSVIEKMNTQFEAVGKEFNNLERRLENLNKKMEDGFLDVWTYNAELLVLMENERTLDSHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 53 - DNA, 1765 bp, synthetic construct
  source 1..1765: mol_type = other DNA
agcaaaagcaggggaaaacaaaagcaacaaaaatgaccatcacttttctcatcctcctgttcacagtagtgaaaggggaccaaatatgtatcggataccatgccaacaattccacagaaaaagttgacacaatcttggaacgaaacgtcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagccaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgtcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactaacaacgtaaattctgtgattgaaaagatgaacactcagtttgaggctgttgggaaagagttcaacaatctagagagaagactagaaaacttaaataagaaaatggaagatggatttctcgatgtatggacatacaatgccgaactcctagttctaatggaaaatgagaggacacttgattcccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgacattaggatttcagaatcatgagaaaaacacccttgtttctact

SEQ ID NO: 54 - protein, 562 aa, synthetic construct
MTITFLILLFTVVKGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKANGQGGRMEFSWTLLETWDVINFESTGNLIAPEYGFKISKRGSSGIMKTEKTLENCETRCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGITNNVNSVIEKMNTQFEAVGKEFNNLERRLENLNKKMEDGFLDVWTYNAELLVLMENERTLDSHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 55 - DNA, 1765 bp, synthetic construct
  source 1..1765: mol_type = other DNA
agcaaaagcaggggaaaacaaaagcaacaaaaatgaccatcacttttctcatcctcctgttcacagtagtgaaaggggaccaaatatgtatcggataccatgccaacaattccacagaaaaagttgacacaatcttggaacgaaacatcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtgggaagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagtcaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgtcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactaacaaggtaaattctgtgattgaaaagatgaacactcagtttgaggctgttgggaaagagttcaacaatctagagagaagactagaaaacttaaataagaaaatggaagatggatttctcaatgtatggacatacaatgccgaactcctagttctaatggaaaatgagaggacacttgatttccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgacattaggatttcagaatcatgagaaaaacacccttgtttctact

SEQ ID NO: 56 - protein, 562 aa, synthetic construct
MTITFLILLFTVVKGDQICIGYHANNSTEKVDTILERNITVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGKQMLIIWGVHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETRCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLNVWTYNAELLVLMENERTLDFHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 57 - DNA, 1765 bp, synthetic construct
  source 1..1765: mol_type = other DNA
agcaaaagcaggggaaaacaaaagcaacaaaaatgaccatcacttttctcatcctcctgttcacagtagtgaaaggggaccaaatatgtatcggataccatgccaacaattccacagaaaaagttgacacaatcttggaacgaaacatcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagtcaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgtcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggacaactaacaaggcaaattctgtgattgaaaagatgaacactcagtttgaggctgttgggaaagagttcaacaatctagagagaagactagaaaacttaaataagaaaatggaagatggatttctcgatgtatggacatacaatgccgaactcctagttctaatggaaaatgagaggacacttgatttccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgacattaggatttcagaatcatgagaaaaacacccttgtttctact

SEQ ID NO: 58 - protein, 562 aa, synthetic construct
MTITFLILLFTVVKGDQICIGYHANNSTEKVDTILERNITVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETRCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGTTNKANSVIEKMNTQFEAVGKEFNNLERRLENLNKKMEDGFLDVWTYNAELLVLMENERTLDFHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 59 - DNA, 1772 bp, synthetic construct
  source 1..1772: mol_type = other DNA
agcaaaagcaggggttataccatagacaaccgaacaaagacaatgaccatcacttttctcatcctcctgttcacagtagtgaaaggggaccaaatatgtatcggataccatgccaacaattccacagaaaaagttgacacaatcttggaacgaaacatcaccgtgactcatgccaaggacattcttgaaaagacgcataatggaaagttgtgcagattaagcgggatccctccattggaactgggggattgcagcattgcaggttggctccttggaaatccagagtgtgaccggctcttaagtgtacctgaatggtcctatatagtggaaaaggaaaacccggtgaatggtctgtgctacccaggcagtttcaatgactatgaggaattgaaacatctcctcaccagtgtgacacactttgagaaagttaagatcctgcctagagatcaatggacccagcacacaacaactggtggttctcgggcctgtgcagtatttgggaatccatcattcttcagaaacatggtctggcttacaaagaaggggtcaaactacccaatcgctaaaaggtcatacaacaacacaagtggggagcaaatgctgataatctggggggtacatcatcccaatgacgatgcagaacaaaggacactgtaccagaatgtgggaacatatgtttccgttggaacatcaacactaaataagaggtcaatccctgaaatagcgacaaggcccaaagtcaatggacaaggagggagaatggaattctcttggactctattagagacatgggatgtcataaattttgagagcactggtaatttaattgcaccagaatatggattcaaaatatcaaagagaggaagctcaggaattatgaagacagagaaaacacttgaaaattgtgagaccagatgccagacccccttaggggcaataaatacaacattgccctttcacaacattcacccattgacaataggtgagtgccccaagtatgtcaagtcagatagactggtcttggcgacaggactaagaaatgttcctcagattgaatcaaggggattgtttggagcaatagctgggtttatagaaggcggatggcaagggatggttgatggctggtatgggtatcatcacagcaatgatcaaggatcaggatatgcagcagacaaagaatccactcaaaaggcaattgacgggataactaacaacgtaaattctgtgattgaaaagatgaacactcagtttgaggctgttgggaaagagttcaacaatctagagagaagactagaaaacttaaataagaaaatggaagatggatttctcaatgtatggacatacaatgccgaactcctagttctaatggaaaatgagaggacacttgatttccatgactctaatgtgaggaatctgtacgataaggtcagaatgcaactgagggacaatgctaaggaaatagggaacggatgctttgagttttatcataaatgtgatgatgaatgcatgaacagtgtcaggaatggaacatatgattatcccaaatatgaagaagagtccaagctgaacaggaacgaaatcaaaggggtgaaattgagcgatatgggggtctatcaaatacttgctatatacgctacagttgcaggctccttgtcactggcaatcatgatagctgggatttctttctggatgtgttctaatgggtctctgcaatgcagaatttgcatttgactgtgagtcaatttgtaattaaaaacacccttgtttctact

SEQ ID NO: 60 - protein, 562 aa, synthetic construct
MTITFLILLFTVVKGDQICIGYHANNSTEKVDTILERNITVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVFGNPSFFRNMVWLTKKGSNYPIAKRSYNNTSGEQMLIIWGVHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETRCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGITNNVNSVIEKMNTQFEAVGKEFNNLERRLENLNKKMEDGFLNVWTYNAELLVLMENERTLDFHDSNVRNLYDKVRMQLRDNAKEIGNGCFEFYHKCDDECMNSVRNGTYDYPKYEEESKLNRNEIKGVKLSDMGVYQILAIYATVAGSLSLAIMIAGISFWMCSNGSLQCRICI

SEQ ID NO: 61 - DNA, 1765 bp, synthetic construct; identical to SEQ ID NO: 35
  source 1..1765: mol_type = other DNA

SEQ ID NO: 62 - protein, 562 aa, synthetic construct; identical to SEQ ID NO: 36

SEQ ID NO: 63 - DNA, 1765 bp, synthetic construct; identical to SEQ ID NO: 35
  source 1..1765: mol_type = other DNA

SEQ ID NO: 64 - protein, 562 aa, synthetic construct; identical to SEQ ID NO: 36

SEQ ID NO: 65 - DNA, 1765 bp, synthetic construct; identical to SEQ ID NO: 37
  source 1..1765: mol_type = other DNA

SEQ ID NO: 66 - protein, 562 aa, synthetic construct; identical to SEQ ID NO: 38

SEQ ID NO: 67 - DNA, 1765 bp, synthetic construct; identical to SEQ ID NO: 37
  source 1..1765: mol_type = other DNA

SEQ ID NO: 68 - protein, 562 aa, synthetic construct; identical to SEQ ID NO: 38

SEQ ID NO: 69 - protein, 230 aa, Alphainfluenzavirus influenzae
MDSNTVSSFQVDCFLWHVRKRFADQELGDAPFLDRLRRDQKSLRGRGSTLGLDIETATRAGKQIVERVLEEESDEALKMTIASVPASRYLTDMTLEEMSRDWFMLMPKQKVAGSLCIRMDQAIMDKNIILKANFSVIFDRLETLILLRAFTEEGAIVGEISPLPSLPGHTDEDVKNAIGVLIGGLEWNDNTVRVSETLQRFAWRSSNEDGRPPLPPKQKRKMARTIESEV

SEQ ID NO: 70 - protein, 121 aa, Alphainfluenzavirus influenzae
MDSNTVSSFQDILMRMSKMQLGSSSEDLNGMITQFESLKLYRDSLGEAVMRMGDLHSLQSRNGKWREQLSQKFEEIRWLIEEVRHRLKITENSFEQITFMQALQLLLEVEQEIRTFSFQLI

SEQ ID NO: 71 - protein, 90 aa, Alphainfluenzavirus influenzae
MEQEQDTPWTQLTEHINTQKRENGQQTQRLEHPNLTQLMDHCPRTMSQVDMHKRTVSLKQWLSLKSLTQESLKTRVLKRWKLFNKQEWTN

SEQ ID NO: 72 - protein, 57 aa, Alphainfluenzavirus influenzae
MEQEQDTPWTQSTEHINIQKKGSGRQTLRLGHPNSIQLMDHYLRIMNQVGMHRQTVF

SEQ ID NO: 73 - DNA, 12 bp, Alphainfluenzavirus influenzae
  source 1..12: mol_type = other DNA
agcraaagcagg

SEQ ID NO: 74 - DNA, 13 bp, Alphainfluenzavirus influenzae
  source 1..13: mol_type = other DNA
ccttgtttctact